(19) United States
(12) Patent Application Publication
(10) Pub. No.: US 2025/0223545 A1
(43) Pub. Date: Jul. 10, 2025

(54) NEW LACTIC ACID BACTERIA

(71) Applicant: INTERNATIONAL N&H DENMARK APS, KONGENS LYNGBY (DK)

(72) Inventors: Armelle COCHU-BLACHÈRE, Dangé-Saint-Romain (FR); Christophe FREMAUX, Dangé-Saint-Romain (FR); Thomas DESFOUGÈRES, Dangé-Saint-Romain (FR); Anaïs JEDRZEJOWSKI, Dangé-Saint-Romain (FR)

(21) Appl. No.: 19/012,114

(22) Filed: Jan. 7, 2025

Related U.S. Application Data
(63) Continuation of application No. 17/415,964, filed on Jun. 18, 2021, now Pat. No. 12,286,618.

(51) Int. Cl. C12N 1/20; A23C 9/123; C07K 14/315; C12N 9/12

(52) U.S. Cl. CPC C12N 1/20; A23C 9/1238; C07K 14/315; C12N 9/1205; C12Y 207/01002; A23V 2400/249

(57) ABSTRACT

The invention relates to a lactose-positive, galactose-negative, Streptococcus thermophilus strain carrying a mutation in 2 or 3 genes selected from the group consisting of 1) a gene encoding a protein of the mannose-glucose-specific PTS and a glcK gene, 2) a gene encoding a protein of the mannose-glucose-specific PTS and a ccpA gene, and 3) a gene encoding a protein of the mannose-glucose-specific PTS, a glcK gene and a ccpA gene, wherein said strain, when used to ferment milk, provides a low lactose fermented milk and/or a fermented milk not undergoing acidification when stored at fermentation temperature.

### CROSS-REFERENCE TO RELATED APPLICATIONS

This application is a U.S. national stage application under 35 U.S.C. § 371 of International Application No. PCT/EP2018/086681, filed on Dec. 21, 2018, entitled “NEW LACTIC ACID BACTERIA,” the contents of which are incorporated by reference in their entirety.

### INCORPORATION BY REFERENCE OF THE SEQUENCE LISTING

The present application is being filed with a Sequence Listing in electronic format. The Sequence Listing is provided as a file entitled 20250327_NB41483USPCN_SequenceListing, created on Mar. 27, 2025, which is 318,836 bytes in size. The information in the electronic format of the Sequence Listing is incorporated by reference in its entirety.

### FIELD OF THE INVENTION

The invention relates to a lactose-positive, galactose-negative, Streptococcus thermophilus strain carrying a mutation in genes selected from the group consisting of 1) a gene encoding a protein of the mannose-glucose-specific PTS and a glcK gene, 2) a gene encoding a protein of the mannose-glucose-specific PTS and a ccpA gene, and 3) a gene encoding a protein of the mannose-glucose-specific PTS, a glcK gene and a ccpA gene, wherein said strain, when used to ferment milk, provides a low lactose fermented milk and/or a fermented milk not undergoing post-acidification when stored at fermentation temperature. The invention also concerns a composition comprising at least one, lactose-positive, galactose-negative, Streptococcus thermophilus strain of the invention, and the use of this strain or composition to manufacture a fermented dairy product

### BACKGROUND TO THE INVENTION

The food industry uses bacteria in order to improve the taste and the texture of food or feed products. In the case of the dairy industry, lactic acid bacteria are commonly used in order to, for example, bring about the acidification of milk (by fermentation of lactose) and to texturize the product into which they are incorporated. For example, the lactic acid bacteria of the species Streptococcus thermophilus (S. thermophilus) are used extensively, alone or in combination with other bacteria, in the manufacture of fresh fermented dairy products, such as cheese or yoghurt.

One of the limitations of the use of lactic acid bacteria in dairy technology is post-acidification, i.e. the production of lactic acid by the lactic acid bacteria after the target pH (the one required by the technology) has been obtained [termination of fermentation]. Thus, the post-acidification phenomenon is not only an issue for the dairy product manufacturers (who would like to have flexible manufacturing process, without necessarily having a rapid cooling step right after the target pH is obtained) but also for the consumers (production of lactic acid bacteria leading to an elevated acidity and reduced shelf life of the fermented product).

In addition, there is a trend from dairy consumers to have fermented products with reduced or low content of lactose (lactose intolerance).

WO2015/193459 proposes several solutions to overcome these issues: controlling the concentration of lactose in the milk before fermentation for example by adding lactase, providing lactic acid bacteria which are not able to hydrolyse lactose (lactose-negative strains). These solutions are however not satisfactory for dairy product manufacturers, since they require either the addition of exogenous enzyme (such as lactase) in the milk before fermentation rendering the manufacturing process more complex and more expensive, or the addition of a carbohydrate into the milk (such as sucrose) what is not in agreement with the growing demand for healthier products with no additives.

Therefore, there is a need for improving methods for producing fermented dairy products, which are both satisfactory for the manufacturers and the consumers, not undergoing post-acidification and with a reduced lactose content.

### DESCRIPTION OF THE DRAWINGS

FIG. 1: Alignment of the GlcK protein sequence of DGCC7710 (DSM28255), DSM32587 (SEQ ID NO: 22) and ST1 m-glcK0-gal+ strains (SEQ ID NO: 23). Differences with the GlcK protein of DGCC7710 (SEQ ID NO:2) are boxed.

FIGS. 2 to 10 are graphs representing the evolution of the pH over time (A) and representing the velocity as a function of the pH (B) of a milk fermented respectively with the DGCC7710 (FIG. 2), ST1 m-glcK+manM (FIG. 3), ST1 m-ccpA+manL (FIG. 4), ST1 m-ccpA+manM (FIG. 5), ST1m-ccpA+manN (FIG. 6), ST1m-glcK+ccpA+manM (FIG. 7), ST1.1 (FIG. 8), ST1.1m-glcK+manM (FIG. 9) or ST1.1m-ccpA+manL (FIG. 10) strain.

### DETAILED DESCRIPTION

The present invention has put in evidence that mutations tightly deregulating sugar metabolism can be used to design Streptococcus thermophilus strains, which can be used to obtain low lactose fermented milk products and/or which can be used to produce fermented milk not undergoing post-acidification even when stored at fermentation temperature.

The inventors have nicely shown that such Streptococcus thermophilus strains can be characterized by the ratio of the amount of galactose released over the amount of lactose remaining in the fermented milk. This ratio translates the ability of the strain to consume lactose (uptake and hydrolysis) and to convert it into free galactose and glucose. Due to the galactose-negative phenotype of the Streptococcus thermophilus strains of the invention, the released galactose represents stoichiometrically the consumed lactose. It should be considered as the efficiency of the strain to over used lactose. Thus, the inventors have shown that in the galactose-negative strains of the invention, the catabolism of carbohydrates coming from lactose hydrolysis is tightly unregulated, such that the consumption of lactose is increased while the strain still keeps an acceptable growth for its use at an industrial level. The strains of the invention do not need to be galactose positive (phenotype which has been shown to be unstable on lactose).

The present invention is directed to a lactose-positive, galactose-negative, Streptococcus thermophilus strain, wherein, when said strain is used to ferment milk as assayed by test B, the ratio of the amount of galactose released (mM) over the amount of remaining lactose (mM) in said fermented milk is more than 1.2, more than 1.5, more than 2 or more than 3.

In an embodiment, the present invention is directed to a lactose-positive, galactose-negative, Streptococcus thermophilus strain carrying a mutation in genes selected from the group consisting of 1) a least one gene encoding a protein of the mannose-glucose-specific PTS and a glcK gene, 2) at least one gene encoding a protein of the mannose-glucose-specific PTS and a ccpA gene, and 3) a gene encoding a protein of the mannose-glucose-specific PTS, a glcK gene and a ccpA gene;
wherein, when said strain is used to ferment milk as assayed by test B, the ratio of the amount of galactose released (mM) over the amount of remaining lactose (mM) in said fermented milk is more than 1.2, more than 1.5, more than 2 or more than 3.

In an embodiment, the lactose-positive, galactose-negative, Streptococcus thermophilus strain carrying a mutation in one gene encoding a protein of the mannose-glucose-specific PTS and a glcK gene. In an embodiment, the gene encoding a protein of the mannose-glucose-specific PTS is selected from the group consisting of the manL gene, the manM gene, the manN gene and the manO gene. In an embodiment, the gene encoding a protein of the mannose-glucose-specific PTS is selected from the group consisting of the manL gene, the manM gene and the manN gene. In an embodiment, the lactose-positive, galactose-negative, Streptococcus thermophilus strain carrying a mutation in one gene selected from the group consisting of manL gene, the manM gene and the manN gene. In an embodiment, the lactose-positive, galactose-negative, Streptococcus thermophilus strain carrying a mutation in 2 genes selected from the group consisting of manL gene, the manM gene and the manN gene. In an embodiment, the lactose-positive, galactose-negative, Streptococcus thermophilus strain carrying a mutation in the manL gene, the manM gene and the manN gene.

In an embodiment, the present invention is directed to a lactose-positive, galactose-negative, Streptococcus thermophilus strain carrying a mutation in 2 or 3 genes selected from the group consisting of 1) a gene encoding a protein of the mannose-glucose-specific PTS and a glcK gene, 2) a gene encoding a protein of the mannose-glucose-specific PTS and a ccpA gene, and 3) a gene encoding a protein of the mannose-glucose-specific PTS, a glcK gene and a ccpA gene;
wherein, when said strain is used to ferment milk as assayed by test B, the ratio of the amount of galactose released (mM) over the amount of remaining lactose (mM) in said fermented milk is more than 1.2, more than 1.5, more than 2 or more than 3.
   The Ratio of Amount of Galactose Released Over the Amount of Remaining Lactose by the Lactose-Positive, Galactose-Negative, Streptococcus thermophilus Strains of the Invention During Milk Fermentation.

In an embodiment, the lactose-positive, galactose-negative, Streptococcus thermophilus strain of the invention exhibit a ratio of the amount of galactose released (mM) over the amount of remaining lactose (mM) in said fermented milk which is more than 1.2. In an embodiment, the ratio of the amount of galactose released (mM) over the amount of remaining lactose (mM) in said fermented milk is selected from the group consisting of more than 1.2, more than 1.5, more than 2 and more than 3. In an embodiment, the ratio of the amount of galactose released (mM) over the amount of remaining lactose (mM) in said fermented milk is more than 1.5. In an embodiment, the ratio of the amount of galactose released (mM) over the amount of remaining lactose (mM) in said fermented milk is more than 2. In an embodiment, the ratio of the amount of galactose released (mM) over the amount of remaining lactose (mM) in said fermented milk is more than 3.

According to the invention, the amount of galactose released (mM) and the amount of remaining lactose (mM) during milk fermentation can be determined by methods well known in the art. In an embodiment, the concentration of galactose and lactose in a fermented milk is characterized by the Test B as defined below:

### Test B:

UHT semi-skimmed milk “Le Petit Vendéen (“yoghurt milk”) containing 3% (w/v) milk powder (BBA, Lactalis), previously pasteurized 10 min at 90° C., is inoculated at 1% (v/v, about 10⁷ CFU/ml) with a culture of the S. thermophilus strain to be assayed (M17-carbohydrate-free resuspended cells from overnight culture grown in M17 supplemented 3% sucrose). This milk is found to contain around 175 mM of lactose. The inoculated milk flasks are statically incubated in a water bath at 43° C. during 24h, to obtain fermented milk. T0 samples and samples of fermented milk (T24h) (5 g) are diluted in 25 g 0.025 N H₂SO₄, before being centrifuged at 4600 rpm for 10 minutes at 4° C. The supernatant is filtered through a 0.2 μm Nylon filter (Phenomenex, Germany, Aschaffenburg) directly into a 2 ml HPLC vial. Samples are stored at −20° C. until further analysis. Carbohydrates [in particular galactose and lactose] are quantified by high performance liquid chromatography (Agilent 1200 HPLC) equipped with a refractive index detector using an AMINEX™ HPX-87H anion exchange column (Bio-Rad Laboratories Inc.) at 35° C., with 12.5 mM H₂SO₄ as the elution fluid and a flow rate of 0.6 ml min⁻¹. The exploitation of results is made with CHEMSTATION® reprocessing software (Agilent).

For the avoidance of doubt, the Streptococcus thermophilus species is to be understood as a Streptococcus salivarius subsp. thermophilus strain.

By the expression “lactose-positive”, it is meant a Streptococcus thermophilus strain which is able to grow on lactose as a sole source of carbohydrate source, in particular on a M17 medium supplemented with 2% lactose. In a particular embodiment, the “lactose-positive” phenotype is assayed by inoculating—into a M17 broth containing 2% lactose—an overnight culture of the S. thermophilus strain to be tested at a rate of 1%, and incubating for 20 hours at 37° C., and wherein a pH of 5.5 or lower at the end of incubation is indicative of a lactose-positive phenotype.

By the expression “galactose-negative”, it is meant a Streptococcus thermophilus strain which is not able to grow on galactose as a sole source of carbohydrate source, in particular on a M17 medium supplemented with 2% galactose. In a particular embodiment, the “galactose-negative” phenotype is assayed by inoculating—into a M17 broth containing 2% galactose—an overnight culture of the S. thermophilus strain to be tested at 1% and incubating for 20 hours at 37° C., and wherein a pH of 6 or above at the end of incubation is indicative of a galactose-negative phenotype.

By the expression “derivative” in reference to an original strain (e.g. DGCC7710-derivative), it is meant a strain obtained from an original strain (e.g. from the DGCC7710 strain) by replacement of one of its genes (such as glcK, ccpA, . . . ) by another allele (in particular a mutated allele) of the same gene. In an embodiment, the derivative is obtained by the replacement of the full gene (coding sequence and promoter) of the original strain by another allele (coding sequence and promoter) of the same gene. In an embodiment, the derivative is obtained by the replacement of the coding sequence of a gene of the original strain by another allele (coding sequence) of the same gene.

Thus, the invention is directed to:
a lactose-positive, galactose-negative, Streptococcus thermophilus strain carrying a mutation in at least one, in particular one, gene encoding a protein of the mannose-glucose-specific PTS and carrying a mutation in the glcK gene, wherein, when said strain is used to ferment milk as assayed by test B, the ratio of the amount of galactose released (mM) over the amount of remaining lactose (mM) in said fermented milk is more than 1.2, more than 1.5, more than 2 or more than 3. In an embodiment, the mutation in the gene encoding a protein of the mannose-glucose-specific PTS reduces or abolishes the import of glucose from the medium into the bacteria. In an embodiment, the mutated glcK gene encodes a glucokinase, the glucokinase activity of which in said strain is significantly reduced but not null. In an embodiment, said lactose-positive, galactose-negative, Streptococcus thermophilus strain carries a mutation in a gene encoding a protein of the mannose-glucose-specific PTS reducing or abolishing the import of glucose from the medium into the bacteria and carrying a mutation in the glcK gene encodes a glucokinase, such that the glucokinase activity of which in said strain is significantly reduced but not null in said strain. In any of these embodiments, the gene encoding a protein of the mannose-glucose-specific PTS is selected from the group consisting of the manL gene, the manM gene and the manN gene;
a lactose-positive, galactose-negative, Streptococcus thermophilus strain carrying a mutation in at least one, in particular one, encoding a protein of the mannose-glucose-specific PTS and carrying a mutation in the ccpA gene, wherein, when said strain is used to ferment milk as assayed by test B, the ratio of the amount of galactose released (mM) over the amount of remaining lactose (mM) in said fermented milk is more than 1.2, more than 1.5, more than 2 or more than 3. In an embodiment, the mutation in the gene encoding a protein of the mannose-glucose-specific PTS reduces or abolishes the import of glucose from the medium into the bacteria. In an embodiment, the mutation in the ccpA gene leads to a lactose-positive, galactose-negative, Streptococcus thermophilus strain exhibiting a ratio of the beta-galactosidase activity of said strain as assayed by test D over the glucokinase activity of said strain as assayed by test E which is at least 4·10⁻⁶, at least 5·10⁻⁶, at least 6·10⁻⁶, at least 7·10⁻⁶ or at least 8·10⁻⁶. In an embodiment, said lactose-positive, galactose-negative, Streptococcus thermophilus strain carries a mutation in a gene encoding a protein of the mannose-glucose-specific PTS reducing or abolishing the import of glucose from the medium into the bacteria and carrying a mutation in the ccpA gene such that the ratio of the beta-galactosidase activity of said strain as assayed by test D over the glucokinase activity of said strain as assayed by test E which is at least 4·10⁻⁶. In any of these embodiments, the gene encoding a protein of the mannose-glucose-specific PTS is selected from the group consisting of the manL gene, the manM gene and the manN gene;
a lactose-positive, galactose-negative, Streptococcus thermophilus strain carrying a mutation in at least one, in particular one, encoding a protein of the mannose-glucose-specific PTS, carrying a mutation in the glcK gene and carrying a mutation in the ccpA gene, wherein, when said strain is used to ferment milk as assayed by test B, the ratio of the amount of galactose released (mM) over the amount of remaining lactose (mM) in said fermented milk is more than 1.2, more than 1.5, more than 2 or more than 3. In an embodiment, the mutation in the gene encoding a protein of the mannose-glucose-specific PTS reduces or abolishes the import of glucose from the medium into the bacteria. In an embodiment, the mutated glcK gene encodes a glucokinase, the glucokinase activity of which in said strain is significantly reduced but not null in said strain. In an embodiment, the mutation in the ccpA gene leads to a lactose-positive, galactose-negative, Streptococcus thermophilus strain exhibiting a ratio of the beta-galactosidase activity of said strain as assayed by test D over the glucokinase activity of said strain as assayed by test E which is at least 4·10⁻⁶, at least 5·10⁻⁶, at least 6·10⁻⁶, at least 7·10⁻⁶ or at least 8·10⁻⁶. In an embodiment, said lactose-positive, galactose-negative, Streptococcus thermophilus strain carries a mutation in a gene encoding a protein of the mannose-glucose-specific PTS reducing or abolishing the import of glucose from the medium into the bacteria, carrying a mutation in the glcK gene encodes a glucokinase, such that the glucokinase activity of which in said strain is significantly reduced but not null in said strain and carrying a mutation in the ccpA gene, such that the ratio of the beta-galactosidase activity of said strain as assayed by test D over the glucokinase activity of said strain as assayed by test E which is at least 4·10⁻⁶. In any of these embodiments, the gene encoding a protein of the mannose-glucose-specific PTS is selected from the group consisting of the manL gene, the manM gene and the manN gene.

The following parts I to III describe respectively mutations of the glcK gene, mutations of the gene encoding a protein of the mannose-glucose-specific PTS (such as mutations of the manL, manM and manN genes) and mutations of the ccpA gene.

Though these mutations are disclosed herein separately (for sake of clarity), any embodiment of one part can be combined with any embodiment of another part or with any embodiment of the two other parts, to design a lactose-positive, galactose-negative, Streptococcus thermophilus strain as defined herein, which, when used to ferment milk as assayed by test B, the ratio of the amount of galactose released (mM) over the amount of remaining lactose (mM) in said fermented milk is more than 1.2, more than 1.5, more than 2 or more than 3.

For the avoidance of doubt, the present invention is directed to a lactose-positive, galactose-negative, Streptococcus thermophilus strain as defined herein, wherein, when said strain is used to ferment milk as assayed by test B, the ratio of the amount of galactose released (mM) over the amount of remaining lactose (mM) in said fermented milk is more than 1.2, more than 1.5, more than 2 or more than 3, wherein said strain carries:
1) a mutation in at least one, in particular one, gene encoding a protein of the mannose-glucose-specific PTS as defined in part II herein and a mutation in its glcK gene as defined in part I herein; or
2) a mutation in at least one, in particular one, gene encoding a protein of the mannose-glucose-specific PTS as defined in part II herein and a mutation in its ccpA gene as defined in part Ill herein; or
3) a mutation in at least one, in particular one, gene encoding a protein of the mannose-glucose-specific PTS as defined in part II herein, a mutation in its glcK gene as defined in part I herein and a mutation in its ccpA gene as defined in part Ill herein;
   I. Mutations of the glcK Gene

This part describes mutations of the glcK gene which can be used either in combination with a mutation of a gene encoding a protein of the mannose-glucose-specific PTS as defined herein, or in combination with a mutation of a gene encoding a protein of the mannose-glucose-specific PTS as defined herein and a mutation of the ccpA gene as defined herein, in the context of a lactose-positive, galactose-negative, Streptococcus thermophilus strain of the invention.

In an embodiment, the mutated glcK gene of the strain of the invention encodes a glucokinase, the glucokinase activity of which in said strain is significantly reduced but not null. Indeed, the inventors have put in evidence that some mutated alleles of the glcK gene codes for a glucokinase (GlcK), the glucokinase activity of which is significantly reduced but not null, when said mutated glcK gene is present in a lactose-positive galactose-negative Streptococcus thermophilus strain.

The expression “glcK gene encoding a glucokinase” means any DNA sequence of a Streptococcus thermophilus strain encoding the glucokinase enzyme which catalyses the conversion of glucose and ATP to glucose-6-phosphate (G6P) and ADP. Non-limitative examples of Streptococcus thermophilus glucokinase sequences are disclosed as SEQ ID Nos:2, 4, 6, 8, 10 12, 14, 16, 18 and 20.

Within the invention, the glucokinase activity in a Streptococcus thermophilus strain is significantly reduced but not null as a consequence of a mutation in its glcK gene. In other words, the allele of the glcK gene carried by said strain is such that the glucokinase activity in said strain is significantly reduced but not null.

The expression “glucokinase activity in said strain is significantly reduced but not null” refers to a strain the glucokinase activity of which is both:
significantly reduced in said strain, in particular as compared to the glucokinase activity in a strain carrying a non-mutated glcK gene; and
not null, i.e., that an activity is detectable by test A as defined herein.

According to the invention, the feature “glucokinase activity in said strain is significantly reduced but not null” can be determined by methods well known in the art. Thus, methods for measuring the glucokinase activity in a Streptococcus thermophilus strain are known and include enzyme assays with commercially available reactants. Reference is made herein to the paragraph 2.4 of Pool et al. (2006. Metabolic Engineering 8(5); 456-464) (incorporated herein by reference). In a particular embodiment, the glucokinase activity in a Streptococcus thermophilus strain of the invention is assayed by test A [i.e. the test A is carried out using the Streptococcus thermophilus strain of the invention] as defined below:

### Test A:

A fresh overnight culture of the Streptococcus thermophilus strain to be assayed in M17 containing 30 g/L lactose is obtained and used to inoculate at 1% (vol/vol) 10 ml of fresh M17 30 g/L lactose. Cells are harvested by centrifugation (6000 g, 10 min, 4° C.) at a 600 nm optical density (OD600) of 0.8+/−0.2, washed in 5 ml cold GLCK buffer (5 mM MgCl2, 10 mM K₂HPO₄/KH₂PO₄ [pH 7.2]), and resuspended in 500 μl cold GLCK buffer. EDTA-free protease inhibitors “cOmplete™” (Roche, supplier reference 04693132001) is added in GLCK buffer as described by the provider. Cells are disrupted by the addition of 100 mg glass beads (150-212 μm, Sigma G1145) to 200 μl resuspended cells and oscillation at a frequency of 30 cycles/s for 6 min in a MM200 oscillating mill (Retsch, Haan, Germany). Cell debris and glass beads are removed by centrifugation (14000 g, 15 min, 4° C.), and supernatant transferred into a clean 1.5 mL centrifuge tube kept on ice. Total protein content is determined by using the FLUKA Protein Quantification Kit-Rapid (ref 51254). The glucokinase activity in the cell extracts is determined spectrophotometrically by a glucose-6-phosphate dehydrogenase (G-6PDH, EC1.1.1.49):NADPH-coupled assay (Porter et al., 1982), essentially as described by Pool et al. (2006). Each sample (5, 10 and 20 μL) is added to assay buffer (10 mM K₂HPO₄/KH₂PO₄ [pH 7.2], 5 mM MgCl2, 1 mM ATP, 20 mM glucose, 1 mM NADP, 1 U G-6PDH) in a 250 μL final volume, and the mixture was left for 5 min at 30° C. The optical density at 340 nm is measured for 5 minutes by using a Synergy HT multi-detection microplate reader (BIO-TEK). One unit of glucokinase corresponds to the amount of enzyme that catalyzes the phosphorylation of 1 μmole of D-glucose to D-Glucose 6-phosphate per minute under the assay conditions. Glucokinase activity is calculated as follows:

$$Glucokinase activity ( U / g of total protein extract ) = dOD \times V / [ dt \times I \times ε \times Qprot ] ,$$

wherein:
dOD is the variation of optical density (OD) at 340 nm
V is the volume of the reaction (herein 250 μL)
dt=measurement time (in minutes)
l=optical path length (herein 0.73 cm)
ε=molar attenuation coefficient of NADPH; H⁺ (herein 6220 cm2/μmol)
Qprot=quantity of protein in the cuvette (in g)
   Measurements are triplicated for each sample, and the glucokinase specific activity values given herein under test A are the mean of three independent experiments.

In a first particular embodiment of the feature “glucokinase activity in said strain is significantly reduced but not null”, the glucokinase activity in the Streptococcus thermophilus strain of the invention is between 200 and 1500 U/g of total protein extract, as assayed by test A. In a particular embodiment, the glucokinase activity in the Streptococcus thermophilus strain of the invention is between 300 and 1200 U/g of total protein extract, as assayed by test A. In a particular embodiment, the glucokinase activity in the Streptococcus thermophilus strain of the invention is between 400 and 1000 U/g of total protein extract, as assayed by test A. In a particular embodiment, the glucokinase activity in the Streptococcus thermophilus strain of the invention is between a minimal value selected from the group consisting of 200, 300 and 400 U/g of total protein extract and a maximal value selected from the group consisting of 1000, 1200 and 1500 U/g of total protein extract, as assayed by test A. It is noteworthy that, as mentioned in test A, the glucokinase activity values disclosed herein are the mean of three independent experiments (triplicates).

In a second particular embodiment of the feature “glucokinase activity in said strain is significantly reduced but not null”, the glucokinase activity in the Streptococcus thermophilus strain of the invention is between 5 and 60% the activity of the glucokinase activity of the DGCC7710 strain deposited at the DSMZ under accession number DSM28255 on Jan. 14, 2014. By “glucokinase activity of the DGCC7710 strain”, it is meant the activity of the DGCC7710 strain glucokinase (i.e., with SEQ ID NO:2) as assayed by test A in the DGCC7710 strain [i.e., the test A is carried out using the DGCC7710 strain]. The percentage value is calculated based on the glucokinase activity in the strain of the invention and the glucokinase activity of the DGCC7710 strain, both assayed by test A. In a particular embodiment, the glucokinase activity in the Streptococcus thermophilus strain of the invention is between 10 and 50% the glucokinase activity of the DGCC7710 strain. In a particular embodiment, the glucokinase activity in the Streptococcus thermophilus strain of the invention is between 15 and 40% the glucokinase activity of the strain DGCC7710. In a particular embodiment, the glucokinase activity of the Streptococcus thermophilus strain of the invention is between a minimal percentage selected from the group consisting of 5, 10 and 15% the glucokinase activity of the DGCC7710 strain and a maximal percentage selected from the group consisting of 40, 50 and 60% the glucokinase activity of the DGCC7710 strain. In a particular embodiment and whatever the range of percentages, the activity of the glucokinase activity is assayed by test A as described herein. It is noteworthy that the percentage values disclosed herein are calculated based on glucokinase activity values which are the mean of three independent experiments (triplicates) as assayed by test A.

In the first and second particular embodiments, the following strains can be used as controls in test A:
as a positive control (i.e., a Streptococcus thermophilus strain, which is representative of strains carrying a non-mutated glcK gene): strain DGCC7710 deposited at the DSMZ under accession number DSM28255 on Jan. 14, 2014;
as a negative control (i.e., a Streptococcus thermophilus strain having no detectable glucokinase activity): either a Streptococcus thermophilus the glcK gene of which is knocked-out or a Streptococcus thermophilus the glcK gene of which carries one of the mutations disclosed in WO2013/160413, WO2017/103051 or Sorensen et al. (2016) and summarized in Table 1 below:

TABLE 1
glcK mutations leading to a strain the glucokinase
activity of which is not detectable

| Mutation | | Mutation | |
|---|---|---|---|
| at the | Change at the | described in | |
| glcK gene | GlcK | the | |
| level | protein level | deposited strain | Strains described in |
| T214C | S72P | DSM25851 | WO2013/160413 |
| C422T | T141I | DSM25850 | WO2013/160413 |
| | | | Sørensen (St1-GS-1) |
| G745A | G249R | DSM28889 | WO2017/103051 |
| | | | Sørensen (St2-GS-1) |

During milk fermentation, the lactose contained in the milk (as the main carbohydrate source in milk) is imported into Streptococcus thermophilus strains. The intracellular lactose is then cleaved into glucose and galactose by the beta-galactosidase enzyme (such that 1 mole of lactose gives 1 mole of glucose and 1 mole of galactose).

The feature “glucokinase activity in said strain is significantly reduced but not null” can also be characterized by the maximum forward velocity of the glucokinase (herein called Vmax, and defined as the velocity of the Glucose+ATP conversion to G6P+ADP) or by the inverse of the affinity of the glucokinase (called Km) for one or two of its substrates, i.e., glucose and ATP. In an embodiment, the feature “glucokinase activity in said strain is significantly reduced but not null” for the strain of the invention is further characterized by the maximum forward velocity (Vmax) of its glucokinase in said strain.

Therefore, in combination with the first or second particular embodiment of the feature “glucokinase activity in said strain is significantly reduced but not null” defined herein, the maximum forward velocity (Vmax) of the glucokinase in the lactose-positive, galactose-negative, Streptococcus thermophilus strain of the invention is significantly reduced but not null. The feature “glucokinase Vmax in said strain is significantly reduced but not null” can be defined by one or two of these parameters:
the Vmax is between 200 and 1500 U/g total protein extract, as assayed by test C.
the Vmax is between 5 and 60% the Vmax of the glucokinase of the DGCC7710 strain deposited at the DSMZ under accession number DSM28255 on Jan. 14, 2014, when both assayed by test C.

In a particular embodiment, the mutated glcK gene of a lactose-positive, galactose-negative, Streptococcus thermophilus strain of the invention encodes a glucokinase, wherein the glucokinase activity in said strain is significantly reduced but not null (as defined herein), and wherein the maximum forward velocity (Vmax) of its glucokinase in said strain is significantly reduced but not null and defined by one or two of these parameters:
the Vmax is between 200 and 1500 U/g total protein extract, as assayed by test C.
the Vmax is between 5 and 60% the Vmax of the glucokinase of the DGCC7710 strain deposited at the DSMZ under accession number DSM28255 on Jan. 14, 2014, when both assayed by test C.

The glucokinase maximum forward velocity (Vmax) in a Streptococcus thermophilus of the invention is assayed by test C [i.e. the test C is carried out using the Streptococcus thermophilus strain of the invention] as defined below:

### Test C:

The maximal forward velocity (Vmax) is determined by using various concentrations of glucose (0, 5, 10, 15, 20 mM) on crude extract prepared as described in test A. Measurements are triplicated for each sample, and the Vmax values given under test C are the mean of three independent experiments. The linear regression representing the inverse of the specific velocity in function of the inverse of the glucose concentration gives the inverse of the maximal forward velocity at the intersection with the Y-axis of the graphic.

In a particular embodiment of the maximum forward velocity of the glucokinase in the Streptococcus thermophilus strain of the invention, the Vmax is between 200 and 1500 U/g total protein extract, as assayed by test C. In a particular embodiment, the Vmax is between 300 and 1200 U/g total protein extract, as assayed by test C. In a particular embodiment, the Vmax is between 400 and 1000 U/g total protein extract. In a particular embodiment, the Vmax of the glucokinase in the Streptococcus thermophilus strain of the invention is between a minimal value selected from the group consisting of 200, 300 and 400 U/g of total protein extract and a maximal value selected from the group consisting of 1000, 1200 and 1500 U/g of total protein extract, as assayed by test C.

In a particular embodiment of the maximum forward velocity of the glucokinase in the Streptococcus thermophilus strain of the invention, the Vmax is between 5 and 60% the Vmax of the glucokinase of the DGCC7710 strain. By “Vmax of the glucokinase of the DGCC7710 strain”, it is meant the Vmax of the DGCC7710 strain glucokinase (i.e., with SEQ ID NO:2) as assayed by test C in the DGCC 7710 strain [i.e., the test C is carried out using the DGCC7710 strain]. The percentage value is calculated based on the Vmax of the glucokinase in the strain of the invention and the Vmax of the DGCC7710 strain, both assayed by test C. In a particular embodiment, the glucokinase Vmax in the Streptococcus thermophilus strain of the invention is between 10 and 50% the Vmax of the glucokinase of the DGCC7710 strain, when both assayed by test C. In a particular embodiment, the glucokinase Vmax in the Streptococcus thermophilus strain of the invention is between 15 and 40% the Vmax of the glucokinase of the DGCC7710 strain. In a particular embodiment, the Vmax of the glucokinase in the Streptococcus thermophilus strain of the invention is between a minimal percentage selected from the group consisting of 5, 10 and 15% the Vmax of the glucokinase activity of the DGCC7710 strain and a maximal percentage selected from the group consisting of 40, 50 and 60% the Vmax of the glucokinase activity of the DGCC7710 strain.

The lactose-positive, galactose-negative Streptococcus thermophilus strain of the invention carries a mutation in the glcK gene encoding a glucokinase, the glucokinase activity of which in said strain is significantly reduced but not null as defined herein and optionally wherein the maximum forward velocity of the glucokinase in said strain is significantly reduced but not null as defined herein.

By “mutation in the glcK gene” within the present invention, it is meant any nucleotide variation within the glcK gene, wherein said variation at the nucleotide level leads to a glucokinase activity in a strain carrying this mutated glcK gene (as the sole glcK gene) which is significantly reduced but not null as defined herein and optionally leads to a maximum forward velocity of the glucokinase in said strain which is significantly reduced but not null as defined herein. In a particular embodiment, by “mutation in the glcK gene” within the present invention, it is meant any nucleotide variation within the open reading frame of the glcK gene, wherein said variation at the nucleotide level leads to a glucokinase activity in a strain carrying this mutated glcK gene (as the sole glcK gene) which is significantly reduced but not null as defined herein and optionally leads to a maximum forward velocity of the glucokinase in said strain which is significantly reduced but not null as defined herein.

Thus, though two Streptococcus thermophilus strains may differ by the sequence of their respective glcK gene, this does not necessarily mean that one of these two glcK genes is mutated in the sense of the invention. Indeed, are not considered as mutations within the present invention:
variations at the nucleotide level which do not lead to any change at the protein level (silent variation) and which do not impact the translation of the glcK RNA; and
variations at the nucleotide level which do lead to a change at the protein level, but wherein this change does not impact the glucokinase activity of the resulting GlcK protein and optionally the maximum forward velocity of the resulting GlcK protein, as defined herein. Indeed, such variations can be observed at the level of the glcK gene of the Streptococcus thermophilus of the invention without impacting the scope of protection.
Non-limitative examples of glcK genes which are not considered as mutated in the sense of the invention are:
the polynucleotide encoding the glucokinase as defined in SEQ ID NO:2 (GlcK type ST1), in particular the polynucleotide as defined in SEQ ID NO:1; this GlcK type is the one of DGCC7710 strain deposited at the DSMZ under accession number DSM28255 on Jan. 14, 2014;
the polynucleotide encoding the glucokinase as defined in SEQ ID NO:4 (GlcK type ST2), in particular the polynucleotide as defined in SEQ ID NO:3;
the polynucleotide encoding the glucokinase as defined in SEQ ID NO:6 (GlcK type ST3), in particular the polynucleotide as defined in SEQ ID NO:5;
the polynucleotide encoding the glucokinase as defined in SEQ ID NO:8 (GlcK type ST4), in particular the polynucleotide as defined in SEQ ID NO:7;
the polynucleotide encoding the glucokinase as defined in SEQ ID NO:10 (GlcK type ST5), in particular the polynucleotide as defined in SEQ ID NO:9;
the polynucleotide encoding the glucokinase as defined in SEQ ID NO:12 (GlcK type ST6), in particular the polynucleotide as defined in SEQ ID NO:11;
the polynucleotide encoding the glucokinase as defined in SEQ ID NO:14 (GlcK type ST7), in particular the polynucleotide as defined in SEQ ID NO:13;
the polynucleotide encoding the glucokinase as defined in SEQ ID NO:16 (GlcK type ST8), in particular the polynucleotide as defined in SEQ ID NO:15;
the polynucleotide encoding the glucokinase as defined in SEQ ID NO:18 (GlcK type ST9), in particular the polynucleotide as defined in SEQ ID NO:17;
the polynucleotide encoding the glucokinase as defined in SEQ ID NO:20 (GlcK type ST10), in particular the polynucleotide as defined in SEQ ID NO:19.
The amino acid differences at the level of the glucokinase together with the percentage of identity of each GlcK type to SEQ ID NO:2 are summarized in Table 3 (example 2).
The glucokinase activity in strains of the GlcK types ST2 to ST10 is summarized in Table 4 (example 3).

Moreover, some nucleotide mutations within the glcK gene are not considered suitable for the purpose of the invention, because they lead to a glucokinase, the activity of which is null or is under the minimal value defined herein, as assayed by test A. Non-limitative examples of non-suitable mutations are described in Table 1. In an embodiment, the Streptococcus thermophilus of the invention does not carry a mutation selected from the group consisting of a mutation leading to the knock-out of the glcK gene and large deletions within the glcK gene.

In an embodiment, the lactose-positive, galactose-negative Streptococcus thermophilus strain of the invention carries a mutation in the open reading frame of the glcK gene leading to the substitution of an amino acid in the GlcK protein, the glucokinase activity of which in said strain carrying the mutated glcK gene is significantly reduced but not null (as defined herein) and optionally wherein the maximum forward velocity of the glucokinase in said strain is significantly reduced but not null as defined herein. In a particular embodiment, the lactose-positive, galactose-negative Streptococcus thermophilus strain of the invention carries a mutation in the glcK gene leading to the substitution of an amino acid in the GlcK protein, the glucokinase activity of which in said strain carrying the mutated glcK gene is significantly reduced but not null (as defined herein) and optionally wherein the maximum forward velocity of the glucokinase in said strain is significantly reduced but not null as defined herein. In a particular embodiment, the Streptococcus thermophilus strain of the invention carries a mutation in the glcK gene such that the GlcK protein is 322-amino acids in length and wherein the glucokinase activity in said strain is significantly reduced but not null as defined herein and optionally wherein the maximum forward velocity of the glucokinase in said strain is significantly reduced but not null as defined herein.

As discussed above, some DNA modifications can be observed at the level of the glcK gene of the Streptococcus thermophilus of the invention which do not impact the glucokinase activity of the strain. Based on test A defined herein together with the control strains defined herein, the person skilled in the art would know how to identify 1) a glcK gene encoding a glucokinase, the glucokinase activity of which in a strain carrying this glcK gene is significantly reduced but not null (as defined herein) and optionally wherein the maximum forward velocity of the glucokinase in a strain carrying this mutated glcK gene is significantly reduced but not null (as defined herein), 2) a glcK gene bearing a modification having no impact on the glucokinase activity in a strain carrying this modification or 3) a glcK gene encoding a glucokinase, the glucokinase activity of which in a strain carrying this glcK gene is null (as defined herein).

The DGCC7710 strain can be used as a control, by replacing its glcK gene by the glcK gene to be assayed to obtain a derivative of DGCC7710, and assaying the DGCC7710 derivative by test A (glucokinase activity) or test C (Vmax).

The inventors have identified two positions within the glucokinase, for which the amino acid nature has been shown to impact the activity of the glucokinase, such that the glucokinase activity is significantly reduced but not null as defined herein and to impact the Vmax of the glucokinase such that the Vmax is significantly reduced but not null as defined herein: position 144 and position 275 of the glucokinase (i.e., codon 144 and 275 of the glcK gene). It is noteworthy that based on tests A and C defined herein together with the control strains, the person skilled in the art would know how to identify other positions and appropriate amino acids within the glucokinase, to obtain a glucokinase activity significantly reduced but not null (as defined herein) and optionally a maximum forward velocity which is significantly reduced but not null, and thus the corresponding glcK gene.

In an embodiment, the amino acid at position 275 of the glucokinase (encoded by the glcK gene of the Streptococcus thermophilus strain of the invention) is not a glutamic acid (i.e., is any amino acid except a glutamic acid); thus, in an embodiment, the codon 275 of the glcK gene carried by the Streptococcus thermophilus strain of the invention is neither GAA nor GAG. In a particular embodiment, the amino acid at position 275 of the glucokinase is not an acidic amino acid (i.e., is any amino acid except an acidic amino acid); thus, in an embodiment, the codon 275 of the glcK gene carried by the Streptococcus thermophilus strain of the invention is a codon encoding a non-acidic amino acid. In a particular embodiment, the amino acid at position 275 of the glucokinase is selected from the group consisting of lysine and any of its conservative amino acids; thus, in an embodiment, the codon 275 of the glcK gene carried by the Streptococcus thermophilus strain of the invention is a codon encoding an amino acid selected from the group consisting of a lysine and any of its conservative amino acids. In a particular embodiment, the amino acid at position 275 of the glucokinase is a lysine; thus, in an embodiment, the codon 275 of the glcK gene carried by the Streptococcus thermophilus strain of the invention is either AAA or AAG. In a particular embodiment, the nucleotides 823-825 of the glcK gene carried by the Streptococcus thermophilus strain of the invention are AAA or AAG.

In a particular embodiment, the sequence of the GlcK protein of the lactose-positive, galactose-negative Streptococcus thermophilus strain of the invention is selected from the group consisting of:
a) a sequence as defined in SEQ ID NO:25, wherein the amino acid at position 275 is any amino acid except a glutamic acid, in particular is any amino acid except an acidic amino acid, in particular is a lysine; and
b) a GlcK variant sequence having at least 90% similarity or identity with SEQ ID NO:25, wherein the amino acid of the glucokinase corresponding to position 275 of SEQ ID NO:25 (or the amino acid at position 275 of the glucokinase) is any amino acid except a glutamic acid, in particular is any amino acid except an acidic amino acid, in particular is a lysine. In a particular embodiment, the GlcK variant sequence is 322-amino acids in length.

In another embodiment, the amino acid at position 144 of the glucokinase (encoded by the glcK gene of the Streptococcus thermophilus strain of the invention) is not a glycine (i.e., is any amino acid except a glycine); thus, in an embodiment, the codon 144 of the glcK gene carried by the Streptococcus thermophilus strain of the invention is not GGT, GGC, GGA or GGG. In a particular embodiment, the amino acid at position 144 of the glucokinase is not an aliphatic amino acid (i.e., is any amino acid except an aliphatic amino acid). In a particular embodiment, the amino acid at position 144 of the glucokinase is selected from the group consisting of serine and any of its conservative amino acids; thus, in an embodiment, the codon 144 of the glcK gene carried by the Streptococcus thermophilus strain of the invention is a codon encoding an amino acid selected from the group consisting of a serine and any of its conservative amino acids. In a particular embodiment, the amino acid at position 144 of the glucokinase is a serine; thus, in an embodiment, the codon 144 of the glcK gene carried by the Streptococcus thermophilus strain of the invention is AGT, AGC, TCT, TCC, TCA or TCG. In a particular embodiment, the nucleotides 430-432 of the glcK gene carried by the Streptococcus thermophilus strain of the invention are AGT, AGC, TCT, TCC, TCA or TCG.

In a particular embodiment, the sequence of the GlcK protein of the lactose-positive, galactose-negative Streptococcus thermophilus strain of the invention is selected from the group consisting of:
a) a sequence as defined in SEQ ID NO:46, wherein the amino acid at position 144 is any amino acid except a glycine, in particular is any amino acid except an aliphatic amino acid, in particular is a serine; and
b) a GlcK variant sequence having at least 90% similarity or identity with SEQ ID NO:46, wherein the amino acid of the glucokinase corresponding to position 144 of SEQ ID NO:46 (or the amino acid at position 144 of the glucokinase) is any amino acid except a glycine, in particular is any amino acid except an aliphatic amino acid, in particular is a serine. In a particular embodiment, the GlcK variant sequence is 322-amino acids in length.

For the definition of the GlcK variant having at least 90% similarity or identity with SEQ ID NO:25, the similarity or identity is calculated herein over the whole length of the 2 sequences after optimal alignment [i.e., number of similar or identical amino acid residues in the aligned parts(s) of the sequences]; the position 275 as defined in SEQ ID NO:25 is not considered for the calculation of the similarity or of the identity. In a particular embodiment, the GlcK variant sequence has at least 91, 92, 93, 94, 95, 96, 97, 98 or 99% similarity or identity with SEQ ID NO:25, wherein the amino acid corresponding to position 275 of SEQ ID NO:25 (or the amino acid at position 275 of the glucokinase) is any amino acid except a glutamic acid, in particular is any amino acid except an acidic amino acid, in particular is a lysine. In an embodiment, the GlcK variant sequence has at least 95% similarity or identity with SEQ ID NO:25, wherein the amino acid corresponding to position 275 of SEQ ID NO:25 (or the amino acid at position 275 of the glucokinase) is any amino acid except a glutamic acid, in particular is any amino acid except an acidic amino acid, in particular is a lysine. In an embodiment, the GlcK variant sequence has at least 97% similarity or identity with SEQ ID NO:25, wherein the amino acid corresponding to position 275 of SEQ ID NO:25 (or the amino acid at position 275 of the glucokinase) is any amino acid except a glutamic acid, in particular is any amino acid except an acidic amino acid, in particular is a lysine.

In a particular embodiment, the GlcK variant sequence differs from SEQ ID NO:25 by from 1 to 30 amino acid substitutions wherein the amino acid at position 275 of said GlcK variant is any amino acid except a glutamic acid, in particular is any amino acid except an acidic amino acid, in particular is a lysine (the position 275 is not considered for the calculation of the number of substitution(s)). In a particular embodiment, the GlcK variant sequence differs from SEQ ID NO:25 by from 1 to 20 amino acid substitutions, wherein the amino acid at position 275 of said GlcK variant is any amino acid except a glutamic acid, in particular is any amino acid except an acidic amino acid, in particular is a lysine. In a particular embodiment, the GlcK variant sequence differs from SEQ ID NO: 25 by from 1 to 15 amino acid substitutions wherein the amino acid at position 275 of said GlcK variant is any amino acid except a glutamic acid, in particular is any amino acid except an acidic amino acid, in particular is a lysine. In a particular embodiment, the GlcK variant sequence differs from SEQ ID NO: 25 by from 1 to 10 amino acid substitutions wherein the amino acid at position 275 of said GlcK variant is any amino acid except a glutamic acid, in particular is any amino acid except an acidic amino acid, in particular is a lysine. In a particular embodiment, the GlcK variant sequence differs from SEQ ID NO: 25 by 1, 2, 3, 4, 5, 6, 7, 8, 9, 10, 11 or 12 amino acid substitutions, wherein the amino acid at position 275 of said GlcK variant is any amino acid except a glutamic acid, in particular is any amino acid except an acidic amino acid, in particular is a lysine.

For the definition of the GlcK variant having at least 90% similarity or identity with SEQ ID NO:46, the similarity or identity is calculated herein over the whole length of the 2 sequences after optimal alignment [i.e., number of similar or identical amino acid residues in the aligned parts(s) of the sequences]; the position 144 as defined in SEQ ID NO:46 is not considered for the calculation of the similarity or of the identity. In a particular embodiment, the GlcK variant sequence has at least 91, 92, 93, 94, 95, 96, 97, 98 or 99% similarity or identity with SEQ ID NO:46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine. In an embodiment, the GlcK variant sequence has at least 95% similarity or identity with SEQ ID NO:46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine. In an embodiment, the GlcK variant sequence has at least 97% similarity or identity with SEQ ID NO:46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine.

In a particular embodiment, the GlcK variant sequence differs from SEQ ID NO:46 by from 1 to 30 amino acid substitutions wherein the amino acid at position 144 of said GlcK variant is any amino acid except a glycine, in particular is any amino acid except an aliphatic amino acid, in particular is a serine (the position 144 is not considered for the calculation of the number of substitution(s)). In a particular embodiment, the GlcK variant sequence differs from SEQ ID NO:46 by from 1 to 20 amino acid substitutions, wherein the amino acid at position 144 of said GlcK variant is any amino acid except a glycine, in particular is any amino acid except an aliphatic amino acid, in particular is a serine. In a particular embodiment, the GlcK variant sequence differs from SEQ ID NO: 46 by from 1 to 15 amino acid substitutions wherein the amino acid at position 144 of said GlcK variant is any amino acid except a glycine, in particular is any amino acid except an aliphatic amino acid, in particular is a serine. In a particular embodiment, the GlcK variant sequence differs from SEQ ID NO: 46 by from 1 to 10 amino acid substitutions wherein the amino acid at position 144 of said GlcK variant is any amino acid except a glycine, in particular is any amino acid except an aliphatic amino acid, in particular is a serine. In a particular embodiment, the GlcK variant sequence differs from SEQ ID NO:46 by 1, 2, 3, 4, 5, 6, 7, 8, 9, 10, 11 or 12 amino acid substitutions, wherein the amino acid at position 144 of said GlcK variant is any amino acid except a glycine, in particular is any amino acid except an aliphatic amino acid, in particular is a serine.

In an embodiment, the sequence of the GlcK protein of the lactose-positive, galactose-negative Streptococcus thermophilus strain of the invention is selected from the group consisting of SEQ ID NOs: 25, 26, 27, 28, 29, 30, 31, 32, 33 and 34, wherein the amino acid at position 275 of said variant is any amino acid except a glutamic acid, in particular is any amino acid except an acidic amino acid, in particular is a lysine; thus, in an embodiment, the glcK gene carried by the Streptococcus thermophilus strain of the invention encodes a GlcK protein, the sequence of which is selected from the group consisting of SEQ ID NOs: 25, 26, 27, 28, 29, 30, 31, 32, 33 and 34, wherein the amino acid at position 275 of the glucokinase is not a glutamic acid, in particular is not an acidic amino acid, in particular is a lysine respectively.

In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is not a glutamic acid; thus, in an embodiment, the codon 275 of the glcK gene carried by the Streptococcus thermophilus strain of the invention is neither GAA nor GAG; thus, in an embodiment, the glcK gene carried by the Streptococcus thermophilus strain of the invention encodes a GlcK protein, the sequence of which is selected from the group consisting of SEQ ID NO: 25 and any GlcK variant sequence having at least 90% similarity or identity with SEQ ID NO:25 as defined herein (in particular SEQ ID NO: 26, 27, 28, 29, 30, 31, 32, 33 or 34), wherein the amino acid of the glucokinase corresponding to position 275 of SEQ ID NO:25 (or the amino acid at position 275 of the glucokinase) is not a glutamic acid.

In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is not an acidic amino acid; thus, in an embodiment, the codon 275 of the glcK gene carried by the Streptococcus thermophilus strain of the invention is a codon which does not encode an acidic amino acid; thus, in an embodiment, the glcK gene carried by the Streptococcus thermophilus strain of the invention encodes a GlcK protein, the sequence of which is selected from the group consisting of SEQ ID NO: 25 and any GlcK variant sequence having at least 90% similarity or identity with SEQ ID NO:25 as defined herein (in particular SEQ ID NO: 26, 27, 28, 29, 30, 31, 32, 33 or 34), wherein the amino acid of the glucokinase corresponding to position 275 of SEQ ID NO:25 (or the amino acid at position 275 of the glucokinase) is not an acidic amino acid.

In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is selected from the group consisting of lysine and any of its conservative amino acids; thus, in an embodiment, the codon 275 of the glcK gene carried by the Streptococcus thermophilus strain of the invention is a codon encoding an amino acid selected from the group consisting of lysine and any of its conservative amino acids; thus, in an embodiment, the glcK gene carried by the Streptococcus thermophilus strain of the invention encodes a GlcK protein, the sequence of which is selected from the group consisting of SEQ ID NO: 25 and any GlcK variant sequence having at least 90% similarity or identity with SEQ ID NO:25 as defined herein (in particular SEQ ID NO: 26, 27, 28, 29, 30, 31, 32, 33 or 34), wherein the amino acid of the glucokinase corresponding to position 275 of SEQ ID NO:25 (or the amino acid at position 275 of the glucokinase) is a lysine and any of its conservative amino acids.

In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is a lysine; thus, in an embodiment, the codon 275 of the glcK gene carried by the Streptococcus thermophilus strain of the invention is a codon encoding a lysine respectively, in particular is AAA or AAG, respectively; thus, in a particular embodiment, the sequence of the GlcK protein of the lactose-positive, galactose-negative Streptococcus thermophilus strain of the invention is selected from the group consisting of SEQ ID NOs: 22, 35, 36, 37, 38, 39, 40, 41, 42 and 43; thus, in an embodiment, the glcK gene carried by the Streptococcus thermophilus strain of the invention encodes a GlcK protein, the sequence of which is selected from the group consisting of SEQ ID NOs: 22, 35, 36, 37, 38, 39, 40, 41, 42 and 43; in a particular embodiment, the glcK gene carried by the Streptococcus thermophilus strain of the invention is as defined in SEQ ID NO:21.

In another embodiment, the sequence of the GlcK protein of the lactose-positive, galactose-negative Streptococcus thermophilus strain of the invention is selected from the group consisting of SEQ ID NOs: 46, 47, 48, 49, 50, 51, 52, 53, 54 and 55, wherein the amino acid at position 144 of said variant is any amino acid except a glycine, in particular is any amino acid except an aliphatic amino acid, in particular is a serine; thus, in an embodiment, the glcK gene carried by the Streptococcus thermophilus strain of the invention encodes a GlcK protein, the sequence of which is selected from the group consisting of SEQ ID NOs: 46, 47, 48, 49, 50, 51, 52, 53, 54 and 55, wherein the amino acid at position 144 of the glucokinase is not a glycine, in particular is not an aliphatic amino acid, in particular is a serine.

In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is not a glycine; thus, in an embodiment, the codon 144 of the glcK gene carried by the Streptococcus thermophilus strain of the invention is not GGT, GGC, GGA or GGG; thus, in an embodiment, the glcK gene carried by the Streptococcus thermophilus strain of the invention encodes a GlcK protein, the sequence of which is selected from the group consisting of SEQ ID NO: 46 and any GlcK variant sequence having at least 90% similarity or identity with SEQ ID NO:46 as defined herein (in particular SEQ ID NO: 47, 48, 49, 50, 51, 52, 53, 54 or 55), wherein the amino acid of the glucokinase corresponding to position 144 of SEQ ID NO:46 (or the amino acid at position 144 of the glucokinase) is not a glycine.

In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is not an aliphatic amino acid; thus, in an embodiment, the codon 144 of the glcK gene carried by the Streptococcus thermophilus strain of the invention is a codon which does not encode an aliphatic amino acid; thus, in an embodiment, the glcK gene carried by the Streptococcus thermophilus strain of the invention encodes a GlcK protein, the sequence of which is selected from the group consisting of SEQ ID NO:46 and any GlcK variant sequence having at least 90% similarity or identity with SEQ ID NO:46 as defined herein (in particular SEQ ID NO: 47, 48, 49, 50, 51, 52, 53, 54 or 55), wherein the amino acid of the glucokinase corresponding to position 144 of SEQ ID NO:46 (or the amino acid at position 144 of the glucokinase) is not an aliphatic amino acid.

In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is selected from the group consisting of serine and any of its conservative amino acids; thus, in an embodiment, the codon 144 of the glcK gene carried by the Streptococcus thermophilus strain of the invention is a codon encoding an amino acid selected from the group consisting of serine and any of its conservative amino acids; thus, in an embodiment, the glcK gene carried by the Streptococcus thermophilus strain of the invention encodes a GlcK protein, the sequence of which is selected from the group consisting of SEQ ID NO: 46 and any GlcK variant sequence having at least 90% similarity or identity with SEQ ID NO:46 as defined herein (in particular SEQ ID NO: 47, 48, 49, 50, 51, 52, 53, 54 or 55), wherein the amino acid of the glucokinase corresponding to position 144 of SEQ ID NO:46 (or the amino acid at position 144 of the glucokinase) is a serine and any of its conservative amino acids.

In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is a serine; thus, in an embodiment, the codon 144 of the glcK gene carried by the Streptococcus thermophilus strain of the invention is a codon encoding a serine, in particular is AAA or AAG; thus, in a particular embodiment, the sequence of the GlcK protein of the lactose-positive, galactose-negative Streptococcus thermophilus strain of the invention is selected from the group consisting of SEQ ID NOs: 45, 56, 57, 58, 59, 60, 61, 62, 63 and 64; thus, in an embodiment, the glcK gene carried by the Streptococcus thermophilus strain of the invention encodes a GlcK protein, the sequence of which is selected from the group consisting of SEQ ID NOs: 45, 56, 57, 58, 59, 60, 61, 62, 63 and 64; in a particular embodiment, the glcK gene carried by the Streptococcus thermophilus strain of the invention is as defined in SEQ ID NO:44.

When defining the sequence of the GlcK protein of the lactose-positive, galactose-negative Streptococcus thermophilus strain of the invention, it is according to the teaching of this application that the glucokinase activity in the strain expressing this GlcK protein is significantly reduced but not null as defined herein and optionally that the Vmax of the glucokinase in this strain is significantly reduced but not null as defined herein.

II. Mutations of a Gene Encoding a Protein of the Mannose-Glucose-Specific PTS, in Particular Mutations of the manL, manM and manN Genes.

This part describes mutations of a gene encoding a protein of the mannose-glucose-specific phosphotransferase system (mannose-glucose-specific PTS), mannose-glucose-specific PTS, in particular mutations of the manL, manM and manN genes, which can be used either in combination with a mutation of a glcK gene as defined herein, or in combination with a mutation of a ccpA gene as defined herein, or in combination with both a mutation of a glcK gene and a mutation of a ccpA gene as defined herein, in the context of a lactose-positive, galactose-negative, Streptococcus thermophilus strain of the invention.

Any mutation in a gene encoding a protein of the mannose-glucose-specific PTS is appropriate, as long as when combined with a mutated glcK gene as defined herein, or combined with a mutation of a ccpA gene as defined herein, or when combined with both a mutated glcK gene and a mutated ccpA gene as defined herein in a lactose-positive, galactose-negative Streptococcus thermophilus strain, the ratio of the amount of galactose released (mM) over the amount of remaining lactose (mM) in said fermented milk is more than 1.2 as defined herein, when said strain is used to ferment milk (by test B).

The inventors have shown that mutations in a gene encoding a protein of the mannose-glucose-specific PTS, in particular in a mutated manL gene, a mutated manM gene, a mutated manN gene or a mutated manO gene, which reduce or abolish the import of glucose from the medium in a lactose-positive, galactose-negative Streptococcus thermophilus strain, are particularly advantageous within the invention. In an embodiment, the mutation of the gene encoding a protein of the mannose-glucose-specific PTS leads to the reduction or abolition of the glucose import activity of the protein encoded by this gene. In an embodiment, the mutated gene is the manL gene and the mutation of the manL gene leads to the reduction or abolition of the glucose import activity of the IIAB^{Man} protein. In an embodiment, the mutated gene is the manM gene and the mutation of the manM gene leads to the reduction or abolition of the glucose import activity of the IIC^{Man} protein. In an embodiment, the mutated gene is the manN gene and the mutation of the manN gene leads to the reduction or abolition of the glucose import activity of the IID^{Man} protein.

In an embodiment, the mutation of the gene encoding a protein of the mannose-glucose-specific PTS, in particular of the manL gene, manM gene or manN gene, is a mutation leading to the knock-out (i.e., the complete disruption) of the gene.

In an embodiment, the mutation of the gene encoding a protein of the mannose-glucose-specific PTS, in particular of the manL gene, manM gene or manN gene, is a mutation of the promoter of the gene, in particular a mutation of the promoter of the gene reducing or inhibiting the transcription of the gene.

In an embodiment, the mutation of the gene encoding a protein of the mannose-glucose-specific PTS, in particular of the manL gene, manM gene or manN gene, is a mutation introduced into the coding sequence of the gene, in particular a mutation leading to the reduction or abolition of the glucose import activity of the protein encoded by the mutated gene, in particular to the reduction or abolition of the glucose import activity of the IIAB^{Man} protein, IIC^{Man} protein or IID^{Man} protein.

In an embodiment, the mutation of the gene encoding a protein of the mannose-glucose-specific PTS, in particular of the manL gene, manM gene or manN gene is a mutation in the coding sequence of the gene, leading to a truncated protein, in particular to a truncated IIAB^{Man} protein, a truncated IIC^{Man} protein or a truncated IID^{Man} protein, in particular to a truncated protein (such as a truncated IIAB^{Man} protein, a truncated IIC^{Man} protein or a truncated IID^{Man} protein) having a reduced or abolished glucose import activity. Whatever the position of the truncation, the mutation introduced into the gene is either a nucleotide substitution leading to a STOP codon or a deletion, insertion or deletion/insertion leading to a frameshift of the open reading frame and a premature STOP codon. In an embodiment, the mutation introduced into the gene is a nucleotide substitution leading to a STOP codon. In an embodiment, the mutation introduced into the gene is a deletion, insertion or deletion/insertion leading to a frameshift of the open reading frame and a premature STOP codon.

Though two Streptococcus thermophilus strains may differ by the sequence of their respective manL, manM or manN gene, this does not necessarily mean that one of these genes is mutated in the sense of the invention. Indeed, are not considered as mutations of the manL, manM or manN gene within the present invention:
variations at the nucleotide level which do not lead to any change at the protein level (silent variation) and which do not impact the translation of the manL, manM or manN RNA; and
variations at the nucleotide level which do lead to a change at the protein level, but wherein this change does not enable, when combined with a mutated glcK gene as defined herein, or combined with a mutation of a ccpA gene as defined herein, or when combined with both a mutated glcK gene and a mutated ccpA in a gene in a lactose-positive, galactose-negative Streptococcus thermophilus strain, to reach a ratio of the amount of galactose released (mM) over the amount of remaining lactose (mM) in said fermented milk more than 1.2 as defined herein, when said strain is used to ferment milk (by test B).

Non-limitative examples of manL, manM and manN genes (respectively encoding the IIAB^{Man} protein, the IIC^{Man} protein and the IID^{Man} protein) which are not considered as mutated in the sense of the invention are:
the polynucleotide encoding the IIAB^{Man} protein as defined in SEQ ID NO:78 (IIAB^{Man} type ST1), in particular the polynucleotide as defined in SEQ ID NO:77; this IIAB^{Man} type is the one of DGCC7710 strain;
the polynucleotide encoding the IIAB^{Man} protein as defined in SEQ ID NO: 80, 82, 84, 86, 88, 90, 92, 94, 96, 98, 100, 102, 104, 106, 108 and 110 (IIAB^{Man} type ST2 to ST17), in particular the polynucleotide as defined in SEQ ID NO: 79, 81, 83, 85, 87, 89, 91, 93, 95, 97, 99, 101, 103, 105, 107 and 109. The sequence of the IIAB^{Man} proteins as defined in SEQ ID NO: 80, 82, 84, 86, 88, 90, 92, 94, 96, 98, 100, 102, 104, 106, 108 and 110 is from 98.4 to 99.6% identical to SEQ ID NO:78;
the polynucleotide encoding the IIC^{Man} protein as defined in SEQ ID NO:130 (IIC^{Man} type ST1), in particular the polynucleotide as defined in SEQ ID NO:129; this IIC^{Man} type is the one of DGCC7710;
the polynucleotide encoding the IIC^{Man} protein as defined in SEQ ID NO:132, 134, 136, 138, 140, 142, 144, 146, 148, 150, 152, 154 and 156 (IIC^{Man} type ST2 to ST14), in particular the polynucleotide as defined in SEQ ID NO:131, 133, 135, 137, 139, 141, 143, 145, 147, 149, 151, 153 and 155. The sequence of the IIC^{Man} proteins as defined in SEQ ID NO: 132, 134, 136, 138, 140, 142, 144, 146, 148, 150, 152, 154 and 156 is from 98.5 to 99.6% identical to SEQ ID NO:130;
the polynucleotide encoding the IID^{Man} protein as defined in SEQ ID NO:167 (IID^{Man} type ST1), in particular the polynucleotide as defined in SEQ ID NO:166; this IID^{Man} type is the one of DGCC7710 strain;
the polynucleotide encoding the IID^{Man} protein as defined in SEQ ID NO:169, 171, 173, 175, 177, 179, 181, 183, 185, 187, 189, 191, 193, 195, 197, 199, 201, 203 and 205 (IID^{Man} type ST2 to ST20), in particular the polynucleotide as defined in SEQ ID NO:168, 170, 172, 174, 176, 178, 180, 182, 184, 186, 188, 190, 192, 194, 196, 198, 200, 202 and 204. The sequence of the IID^{Man} proteins as defined in SEQ ID NO: 169, 171, 173,175, 177, 179, 181, 183, 185, 187, 189, 191, 193, 195,197, 199, 201, 203 and 205 is from 97.3 to 99.6% identical to SEQ ID NO:167.

The inventors have identified at least one mutation in the manL gene, which when inserted into the manL gene of an original lactose-positive, galactose-negative, Streptococcus thermophilus strain [mutated in the glcK gene, the ccpA gene or both the glcK and ccpA genes as defined herein] enables, when said strain is used to ferment milk, to reach a ratio of the amount of galactose released (mM) over the amount of remaining lactose (mM) in said fermented milk which is more than 1.2 as defined herein (as assayed by test B).

In an embodiment, the mutation in the manL gene leads to the truncation of the IIAB^{Man} protein at position 305. In an embodiment, the mutation in the manL gene is the substitution of the nucleotide G in the nucleotide T at position 916 (leading to a stop codon at position 306). A Streptococcus thermophilus IIAB^{Man} protein truncated at position 305 is referred herein as IIAB^{Man}₃₀₅.

In an embodiment, the sequence of said IIAB^{Man} protein truncated in position 305 is selected from the group consisting of:
a) a sequence as defined in SEQ ID NO:112; and
b) a IIAB^{Man} variant sequence having at least 90% similarity or identity with SEQ ID NO:112, in particular being 305 amino acids in length.

For the definition of the IIAB^{Man} variant having at least 90% similarity or identity with SEQ ID NO:112, the similarity or identity is calculated herein over the whole length of the 2 sequences after optimal alignment [i.e., number of similar or identical amino acid residues in the aligned parts(s) of the sequences]. In a particular embodiment, the IIAB^{Man} variant sequence has at least 91, 92, 93, 94, 95, 96, 97, 98 or 99% similarity or identity with SEQ ID NO: 112.

In a particular embodiment, the IIAB^{Man} variant sequence differs from SEQ ID NO:112 by from 1 to 30 amino acid substitutions. In a particular embodiment, the IIAB^{Man} variant sequence differs from SEQ ID NO: 112 by from 1 to 20 amino acid substitutions. In a particular embodiment, the IIAB^{Man} variant sequence differs from SEQ ID NO: 112 by from 1 to 15 amino acid substitutions. In a particular embodiment, the IIAB^{Man} variant sequence differs from SEQ ID NO: 112 by from 1 to 10 amino acid substitutions. In a particular embodiment, the IIAB^{Man} variant sequence differs from SEQ ID NO: 112 by 1, 2, 3, 4, 5, 6, 7, 8, 9 or 10 amino acid substitutions. In an embodiment, the sequence of the IIAB^{Man} protein of the lactose-positive, galactose-negative Streptococcus thermophilus strain of the invention is selected from the group consisting of SEQ ID NOs: 112 to 128.

In an embodiment, the manL gene carried by the Streptococcus thermophilus strain of the invention encodes a IIAB^{Man} protein, the sequence of which is selected from the group consisting of SEQ ID NO: 112 and any IIAB^{Man} variant sequence having at least 90% similarity or identity with SEQ ID NO: 112 as defined herein (in particular SEQ ID NO:113 to 128). In an embodiment, the manL gene carried by the Streptococcus thermophilus strain of the invention is as defined in SEQ ID NO:111.

The inventors have identified at least one mutation in the manM gene, which when inserted into the manL gene of an original lactose-positive, galactose-negative, Streptococcus thermophilus strain [mutated in the glcK gene, the ccpA gene or both the glcK and ccpA genes as defined herein] enables, when said strain is used to ferment milk, to reach a ratio of the amount of galactose released (mM) over the amount of remaining lactose (mM) in said fermented milk more than 1.2 as defined herein (as assayed by test B).

In an embodiment, the mutation in the manM gene leads to the truncation of the IIC^{Man} protein at position 208. In an embodiment, the mutation in the manM gene is the substitution of the nucleotide G in the nucleotide T at position 625 (leading to a stop codon at position 209). A Streptococcus thermophilus IIC^{Man} protein truncated at position 208 is referred herein as IIC^{Man}₂₀₈ In an embodiment, the sequence of said IIC^{Man} protein truncated in position 208 is selected from the group consisting of:
a) a sequence as defined in SEQ ID NO:158; and
b) a IIC^{Man} variant sequence having at least 90% similarity or identity with SEQ ID NO:158, in particular being 208 amino acids in length.

For the definition of the IIC^{Man} variant having at least 90% similarity or identity with SEQ ID NO:158, the similarity or identity is calculated herein over the whole length of the 2 sequences after optimal alignment [i.e., number of similar or identical amino acid residues in the aligned parts(s) of the sequences]. In a particular embodiment, the IIC^{Man} variant sequence has at least 91, 92, 93, 94, 95, 96, 97, 98 or 99% similarity or identity with SEQ ID NO: 158.

In a particular embodiment, the IIC^{Man} variant sequence differs from SEQ ID NO:158 by from 1 to 30 amino acid substitutions. In a particular embodiment, the IIC^{Man} variant sequence differs from SEQ ID NO:158 by from 1 to 20 amino acid substitutions. In a particular embodiment, the IIC^{Man} variant sequence differs from SEQ ID NO:158 by from 1 to 15 amino acid substitutions. In a particular embodiment, the IIC^{Man} variant sequence differs from SEQ ID NO:158 by from 1 to 10 amino acid substitutions. In a particular embodiment, the IIC^{Man} variant sequence differs from SEQ ID NO:158 by 1, 2, 3, 4, 5, 6, 7, 8, 9 or 10 amino acid substitutions. In an embodiment, the sequence of the IIC^{Man} protein of the lactose-positive, galactose-negative Streptococcus thermophilus strain of the invention is selected from the group consisting of SEQ ID NO:158 to 165.

In an embodiment, the manM gene carried by the Streptococcus thermophilus strain of the invention encodes a IIC^{Man} protein, the sequence of which is selected from the group consisting of SEQ ID NO:158 and any IIC^{Man} variant sequence having at least 90% similarity or identity with SEQ ID NO:158 as defined herein (in particular SEQ ID NO:159 to 165). In an embodiment, the manM gene carried by the Streptococcus thermophilus strain of the invention is as defined in SEQ ID NO:157.

The inventors have identified at least one mutation in the manN gene, which when inserted into the manN gene of an original lactose-positive, galactose-negative, Streptococcus thermophilus strain [mutated in the glcK gene, the ccpA gene or both the glcK and ccpA genes as defined herein] enables, when said strain is used to ferment milk, to reach a ratio of the amount of galactose released (mM) over the amount of remaining lactose (mM) in said fermented milk more than 1.2 as defined herein (as assayed by test B).

In an embodiment, the mutation in the manN gene leads to the truncation of the IID^{Man} protein at position 28. In an embodiment, the mutation in the manN gene is an insertion of a nucleotide A in the stretch of 5 nucleotides A at positions 37-41 (leading to a stretch of 6 nucleotides A, a frameshift of the open reading frame and a truncation of the IID^{Man} protein at position 28). This Streptococcus thermophilus IID^{Man} protein truncated at position 28 is referred herein as IID^{Man}₂₈.

In an embodiment, the sequence of said IID^{Man} protein truncated in position 28 is selected from the group consisting of:
a) a sequence as defined in SEQ ID NO:207; and
b) a IID^{Man} variant sequence having at least 90% similarity or identity with SEQ ID NO:207, in particular being 28 amino acids in length.

For the definition of the IID^{Man} variant having at least 90% similarity or identity with SEQ ID NO:207, the similarity or identity is calculated herein over the whole length of the 2 sequences after optimal alignment [i.e., number of similar or identical amino acid residues in the aligned parts(s) of the sequences]. In a particular embodiment, the IIC^{Man} variant sequence has at least 91, 92, 93, 94, 95, 96, 97, 98 or 99% similarity or identity with SEQ ID NO:207.

In a particular embodiment, the IID^{Man} variant sequence differs from SEQ ID NO:207 by from 1 to 10 amino acid substitutions. In a particular embodiment, the IID^{Man} variant sequence differs from SEQ ID NO:207 by 1, 2, 3, 4, 5, 6, 7, 8, 9 or 10 amino acid substitutions. In an embodiment, the sequence of the IID^{Man} protein of the lactose-positive, galactose-negative Streptococcus thermophilus strain of the invention is selected from the group consisting of SEQ ID NO:207 to 211.

In an embodiment, the manN gene carried by the Streptococcus thermophilus strain of the invention encodes a IID^{Man} protein, the sequence of which is selected from the group consisting of SEQ ID NO:207 and any IID^{Man} variant sequence having at least 90% similarity or identity with SEQ ID NO:207 as defined herein (in particular SEQ ID NO: 208 to 211). In an embodiment, the manN gene carried by the Streptococcus thermophilus strain of the invention is as defined in SEQ ID NO:206.

Whatever the mutation of the glcK gene as defined herein and the mutation of the ccpA gene as defined herein (present alone or combined in a lactose-positive, galactose-negative, Streptococcus thermophilus strain of the invention), at least one gene encoding a protein of the mannose-glucose-specific PTS is mutated as defined herein. Whatever the embodiment, the invention encompasses a lactose-positive, galactose-negative, Streptococcus thermophilus strain carrying a mutation in one, two or three genes selected from the group consisting of the manL gene, the manM gene and the manN gene. In an embodiment, the lactose-positive, galactose-negative, Streptococcus thermophilus strain of the invention carries a mutation in manL. In an embodiment, the lactose-positive, galactose-negative, Streptococcus thermophilus strain of the invention carries a mutation in manM. In an embodiment, the lactose-positive, galactose-negative, Streptococcus thermophilus strain of the invention carries a mutation in manN. In an embodiment, the lactose-positive, galactose-negative, Streptococcus thermophilus strain of the invention carries a mutation in manL and a mutation in manM. In an embodiment, the lactose-positive, galactose-negative, Streptococcus thermophilus strain of the invention carries a mutation in manL and a mutation in manN. In an embodiment, the lactose-positive, galactose-negative, Streptococcus thermophilus strain of the invention carries a mutation in manM and a mutation in manN. In an embodiment, the lactose-positive, galactose-negative, Streptococcus thermophilus strain of the invention carries a mutation in manL, a mutation in manM and a mutation in manN.

Any method can be used to identify a mutation in a gene encoding a protein of the mannose-glucose-specific PTS, in particular in the manL gene, manM gene or manN gene suitable within the lactose-positive, galactose-negative, Streptococcus thermophilus strain of the invention.

As an example, to identify a suitable mutation in the manL gene, manM gene or manN gene, the person skilled in the art can proceed by the following method:
a) provide the DSM32587 strain (mutated in its glcK gene)
b) carry out mutagenesis on the manL, manM or manN gene of the strain in a), for example by random or directed mutagenesis, to obtain a manL, manM or manN gene, the sequence of which is different from the sequence of the manL, manM or manN gene of DSM32587, to obtain a man-mutated DSM32587 strain;
c) determining by test B the ratio of the amount of galactose released (mM) over the amount of remaining lactose (mM) in milk fermented with the man-mutated DSM32587 strain of step b), wherein a ratio of more than 1.2 means that the mutated manL, manM or manN gene is according to the invention. In a particular embodiment, a mutated manL, manM or manN gene is considered to be according to the invention when the ratio of the amount of galactose released (mM) over the amount of remaining lactose (mM) in the fermented milk is more than 1.5, is more than 2 or is more than 3.

Alternatively, the person skilled in the art can proceed by the following method:
a) provide a DGCC7710 strain in which its ccpA gene has been replaced by the mutated ccpA gene as defined in SEQ ID NO:71 (ccpA_{Δ1A114-120}), called herein DGCC7710-ccpA_{Δ1A114-120} strain;
b) carry out mutagenesis on the manL, manM or manN gene of the strain in a), for example by random or directed mutagenesis, to obtain a manL, manM or manN gene, the sequence of which is different from the sequence of the manL, manM or manN gene of the DGCC7710-ccpA_{Δ1A114-120} strain, to obtain a man-mutated DGCC7710-ccpA_{Δ1A114-120} strain;
c) determining by test B the ratio of the amount of galactose released (mM) over the amount of remaining lactose (mM) in milk fermented with the man-mutated DGCC7710-ccpA_{Δ1A114-120} strain of step b), wherein a ratio of more than 1.2 means that the mutated manL, manM or manN gene is according to the invention. In a particular embodiment, a mutated manL, manM or manN gene is considered to be according to the invention when the ratio of the amount of galactose released (mM) over the amount of remaining lactose (mM) in the fermented milk is more than 1.5, is more than 2 or is more than 3.

Once identified, the mutated manL, manM or manN gene according to the invention can be introduced in lieu of the manL, manM or manN of a lactose-positive, galactose-negative, Streptococcus thermophilus strain, to obtain a lactose-positive, galactose-negative, Streptococcus thermophilus strain of the invention.

III. Mutations of the ccpA Gene

This part describes mutations of the ccpA (Catabolite Control Protein A) gene which can be used either in combination with a mutation of a gene encoding a protein of the mannose-glucose-specific PTS as defined herein, or in combination with a mutation of a gene encoding a protein of the mannose-glucose-specific PTS as defined herein and a mutation of the glcK gene as defined herein, in the context of a lactose-positive, galactose-negative, Streptococcus thermophilus strain of the invention.

Any mutation in the ccpA gene is appropriate, as long as when combined with a mutated glcK gene as defined herein, or combined with a mutated gene encoding a protein of the mannose-glucose-specific PTS as defined herein, or when combined with both a mutated glcK gene and a mutated gene encoding a protein of the mannose-glucose-specific PTS as defined herein in a lactose-positive, galactose-negative Streptococcus thermophilus strain, the ratio of the amount of galactose released (mM) over the amount of remaining lactose (mM) in said fermented milk is more than 1.2 as defined herein, when said strain is used to ferment milk.

The inventors have shown that mutations of the ccpA gene, leading to a Streptococcus thermophilus strain of the invention, can be characterized by the ratio of the beta-galactosidase activity as assayed by test D over the glucokinase activity as assayed by test E, in a strain carrying this mutated ccpA. Thus, a ccpA mutation as defined herein is a mutation of the ccpA gene which leads to a lactose-positive, galactose-negative, Streptococcus thermophilus strain exhibiting a ratio of the beta-galactosidase activity as assayed by test D over the glucokinase activity as assayed by test E which is at least 4·10⁻⁶. In an embodiment, the ratio of the beta-galactosidase activity as assayed by test D over the glucokinase activity as assayed by test E is selected from the group consisting of at least 4·10⁻⁶, at least 5·10⁻⁶, at least 6·10⁻⁶, at least 7·10⁻⁶ or at least 8·10⁻⁶. In an embodiment, the ratio of the beta-galactosidase activity as assayed by test D over the glucokinase as assayed by test E is at least 5·10⁻⁶. In an embodiment, the ratio of the beta-galactosidase activity as assayed by test D over the glucokinase as assayed by test E is at least 6·10⁻⁶. In an embodiment, the ratio of the beta-galactosidase activity as assayed by test D over the glucokinase as assayed by test E is at least 7·10⁻⁶. In an embodiment, the ratio of the beta-galactosidase activity as assayed by test D over the glucokinase as assayed by test E is at least 8·10⁻⁶. Whatever the minimal value of the ratio as defined herein, the ratio of the beta-galactosidase activity as assayed by test D over the glucokinase activity as assayed by test E is no less than 8·10⁻³.

For the determination of the ratio of the beta-galactosidase activity over the glucokinase activity in the strain of the invention, test D and test E as described herein are used:

### Test D:

The beta-galactosidase activity in a Streptococcus thermophilus strain of the invention is assayed by test D [i.e. the test D is carried out using the Streptococcus thermophilus strain of the invention].

A fresh overnight culture of the Streptococcus thermophilus strain to be assayed in M17 containing 30 g/L lactose is obtained and used to inoculate at 1% (vol/vol) 10 ml of fresh M17 30 g/L lactose. Cells are harvested by centrifugation (6000 g, 10 min, 4° C.) after 3 hours of growth on M17+30 g/l lactose at 42° C., washed in 1.5 ml cold lysis buffer (KPO4 0.1 M), and resuspended in 300 μl cold lysis buffer. EDTA-free protease inhibitors “cOmplete™” (Roche, supplier reference 04693132001) is added in lysis buffer as described by the provider. Cells are disrupted by the addition of 100 mg glass beads (150-212 μm, Sigma G1145) to 250 μl resuspended cells and oscillation at a frequency of 30 cycles/s for 6 min in a MM200 oscillating mill (Retsch, Haan, Germany). Cell debris and glass beads are removed by centrifugation (14000 g, 15 min, 4° C.), and supernatant transferred into a clean 1.5 mL centrifuge tube kept on ice. Total protein content is determined by using the FLUKA Protein Quantification Kit-Rapid (ref 51254). The beta-galactosidase activity in the cell extracts is determined spectrophotometrically by a monitoring of the hydrolysis of O-nitro-Phenol-Beta-Galactoside (ONPG) into galactose and O-nitro-phenol (ONP). 20 μL of the bacteria extract are mixed with 135 μL of React Buffer (NaPO₄ 0.1 M+KCl 0.01 M+MgSO₄ 0.001 M+ONPG 3 mM+Beta Mercapto Ethanol 60 mM, pH=6). The production of ONP leads to a yellow color into the tube. When this color appears, the reaction is block by adding 250 μL of Stopping buffer (Na₂CO₃ 1 M). The optical density at 420 nm is measured using a Synergy HT multi-detection microplate reader (BIO-TEK). One unit of galactosidase corresponds to the amount of enzyme that catalyzes the production of 1 μmole ONP per minute under the assay conditions. Beta-Galactosidase activity is calculated as follows:

Beta-Galactosidase activity (U/g of total protein extract)=dOD×V/[dt×l×ε×Qprot], wherein:
dOD is the variation of optical density (OD) at 420 nm between the blank and the tested sample
V is the volume of the reaction in which the optical density is measured (herein 250 μL)
dt=represent the duration in minutes between the addition of the 20 μL of bacterial extract and the addition of the 250 μL stopping buffer
l=optical path length (herein 0.73 cm)
ε=molar attenuation coefficient of ONP (herein 4500 cm²/μmol)
Qprot=quantity of protein in the cuvette (in g)
   Measurements are at least triplicated for each sample, and the beta-galactosidase specific activity values given herein under test D are the mean of three independent experiments.

### Test E

The glucokinase activity in a Streptococcus thermophilus strain of the invention, for the determination of the ratio, is assayed by test E [i.e. the test E is carried out using the Streptococcus thermophilus strain of the invention].

A fresh overnight culture of the Streptococcus thermophilus strain to be assayed in M17 containing 30 g/L lactose is obtained and used to inoculate at 1% (vol/vol) 10 ml of fresh M17 30 g/L lactose. Cells are harvested by centrifugation (6000 g, 10 min, 4° C.) after 3 hour of growth on M17+30 g/L lactose at 42° C., washed in 1.5 ml cold GLCK buffer (5 mM MgCl2, 10 mM K₂HPO₄/KH₂PO₄ [pH 7.2]), and resuspended in 300 μl cold GLCK buffer. EDTA-free protease inhibitors “cOmplete™” (Roche, supplier reference 04693132001) is added in GLCK buffer as described by the provider. Cells are disrupted by the addition of 100 mg glass beads (150-212 μm, Sigma G1145) to 250 μl resuspended cells and oscillation at a frequency of 30 cycles/s for 6 min in a MM200 oscillating mill (Retsch, Haan, Germany). Cell debris and glass beads are removed by centrifugation (14000 g, 15 min, 4° C.), and supernatant transferred into a clean 1.5 mL centrifuge tube kept on ice. Total protein content is determined by using the FLUKA Protein Quantification Kit-Rapid (ref 51254). The glucokinase activity in the cell extracts is determined spectrophotometrically by a glucose-6-phosphate dehydrogenase (G-6PDH, EC1.1.1.49):NADPH-coupled assay (Porter et al., 1982), essentially as described by Pool et al. (2006). Each sample (5, 10 and 20 μL) is added to assay buffer (10 mM K₂HPO₄/KH₂PO₄ [pH 7.2], 5 mM MgCl2, 1 mM ATP, 20 mM glucose, 1 mM NADP, 1 U G-6PDH) in a 250 μL final volume, and the mixture was left for 5 min at 30° C. The optical density at 340 nm is measured for 5 minutes by using a Synergy HT multi-detection microplate reader (BIO-TEK). One unit of glucokinase corresponds to the amount of enzyme that catalyzes the phosphorylation of 1 μmole of D-glucose to D-Glucose 6-phosphate per minute under the assay conditions. Glucokinase activity is calculated as follows:

Glucokinase activity (U/g of total protein extract)=dOD×V/[dt×l×ε×Qprot], wherein:
dOD is the variation of optical density (OD) at 340 nm
V is the volume of the reaction (herein 250 μL)
dt=measurement time (in minutes)
l=optical path length (herein 0.73 cm)
ε=molar attenuation coefficient of NADPH; H⁺ (herein 6220 cm2/μmol)
Qprot=quantity of protein in the cuvette (in g)
   Measurements are triplicated for each sample, and the glucokinase specific activity values given herein under test E are the mean of three independent experiments.

In an embodiment, the ccpA gene mutation is not a mutation leading to the knock-out (i.e., the complete disruption) of the gene.

In an embodiment, the ccpA gene mutation is a mutation in the coding sequence of the ccpA gene, in particular in the first 270 nucleotides of the coding sequence of the ccpA gene. In an embodiment, the mutation is a mutation selected from the group consisting of:
a) a non-sense mutation (i.e. leading to a STOP codon) located between the nucleotide 1 and the nucleotide 270 of the coding sequence of the ccpA gene; and
b) a mutation, located in the first quarter of the coding sequence of the ccpA gene (i.e., between nucleotide 1 and the nucleotide 250), leading to a frameshift of the open reading frame of the ccpA gene.

In an embodiment, the mutation leading to a frameshift of the open reading frame of the ccpA gene is located between nucleotide 50 and the nucleotide 200 of the coding sequence of the ccpA gene. In an embodiment, the mutation leading to a frameshift of the open reading frame of the ccpA gene is located between nucleotide 100 and the nucleotide 150 of the coding sequence of the ccpA gene. Whatever the location of the mutation leading to a frameshift, the mutation is selected from the group consisting of a deletion, an insertion or a deletion/insertion (which all are not a multiple of 3).

Though two Streptococcus thermophilus strains may differ by the sequence of their respective ccpA gene, this does not necessarily mean that one of these two ccpA genes is mutated in the sense of the invention. Indeed, are not considered as mutations of the ccpA gene within the present invention:
variations at the nucleotide level which do lead to a change at the protein level, but wherein this change does not enable to obtain a ratio—of the beta-galactosidase activity as assayed by test D over the glucokinase activity as assayed by test E of the lactose-positive, galactose-negative, Streptococcus thermophilus strain encoding this modified protein—of at least 4·10⁻⁶

Non-limitative examples of ccpA genes which are not considered as mutated in the sense of the invention are:
the polynucleotide as defined in SEQ ID NO:65 (ccpA type ST1); this ccpA type is the one of the DGCC7710 strain;
the polynucleotide as defined in SEQ ID NO:66 (ccpA type ST2), which has 99.8% identity with SEQ ID NO:65;
the polynucleotide as defined in SEQ ID NO:67 (ccpA type ST3), which has 99.8% identity with SEQ ID NO:65;
the polynucleotide as defined in SEQ ID NO:68 (ccpA type ST4), which has 99.7% identity with SEQ ID NO:65;
the polynucleotide as defined in SEQ ID NO:69 (ccpA type ST5), which has 99.8% identity with SEQ ID NO:65; and
the polynucleotide as defined in SEQ ID NO:70 (ccpA type ST6), which has 99.7% identity with SEQ ID NO:65.

The inventors have identified at least one mutation, which when present into the ccpA gene of a lactose-positive, galactose-negative, Streptococcus thermophilus strain enables this strain to exhibit a ratio of the beta-galactosidase activity in said strain as assayed by test D over the glucokinase activity in said strain as assayed by test E of at least 4·10⁻⁶ as defined herein. Thus, the invention is directed to a lactose-positive, galactose-negative, Streptococcus thermophilus strain carrying a mutation in the ccpA gene selected from the group consisting of a non-sense mutation located between the nucleotide 1 and the nucleotide 270 of the coding sequence of the ccpA gene and a mutation, located in the first quarter of the coding sequence of the ccpA gene, leading to a frameshift of the open reading frame of the ccpA gene, wherein the ratio of the beta-galactosidase activity in said strain as assayed by test D over the glucokinase activity in said strain as assayed by test E is at least 4·10⁻⁶ as defined herein.

In an embodiment, the mutation of the ccpA gene is a deletion of a nucleotide A in the stretch of 7 nucleotides A at positions 114-120 (leading to a frameshift of the open reading frame of the ccpA gene). Such Streptococcus thermophilus mutated ccpA gene is referred herein as ccpA_{Δ1A114-120}.

In an embodiment, the sequence of said ccpA gene with a STOP codon at codon 66 is selected from the group consisting of:
a) a sequence as defined in SEQ ID NO:71; and
b) a ccpA variant sequence having at least 90% identity with SEQ ID NO:71. The ccpA variant as defined herein carries a mutation as defined above, i.e., is selected from the group consisting of a non-sense mutation located between the nucleotide 1 and the nucleotide 270 of the coding sequence of the ccpA gene and a mutation, located in the first quarter of the coding sequence of the ccpA, leading to a frameshift of the open reading frame of the ccpA gene.

For the definition of the ccpA variant having at least 90% identity with SEQ ID NO:71, the identity is calculated herein over the whole length of the 2 sequences after optimal alignment [i.e., number of identical nucleotides in the aligned parts(s) of the sequences]. In a particular embodiment, the ccpA variant sequence has at least 91, 92, 93, 94, 95, 96, 97, 98 or 99% identity with SEQ ID NO:71. In a particular embodiment, the ccpA variant sequence differs from SEQ ID NO:71 by from 1 to 30 nucleotide substitutions. In a particular embodiment, the ccpA variant sequence differs from SEQ ID NO:71 by from 1 to 20 nucleotide substitutions. In a particular embodiment, the ccpA variant sequence differs from SEQ ID NO:71 by from 1 to 15 nucleotide substitutions. In a particular embodiment, the ccpA variant sequence differs from SEQ ID NO:71 by from 1 to 10 nucleotide substitutions. In a particular embodiment, the ccpA variant sequence differs from SEQ ID NO:71 by 1, 2, 3, 4, 5, 6, 7, 8, 9 or 10 nucleotide substitutions. In an embodiment, the sequence of the ccpA gene of the lactose-positive, galactose-negative Streptococcus thermophilus strain of the invention is selected from the group consisting of SEQ ID NOs: 71, 72, 73, 74, 75 and 76.

The person skilled in the art is given, in this part of the application, guidance on how to identify mutations of the ccpA gene other than the one specifically disclosed. Based on the ratio defined above [of the amount of galactose released (mM) over the amount of remaining lactose (mM) in the fermented milk as defined herein] together with a reference strain defined herein, the person skilled in the art would know how to identify a mutated ccpA gene according to the invention and to obtain a Streptococcus thermophilus strain of the invention.

Thus, the person skilled in the art can proceed by the following method:
a) provide a DGCC7710 strain in which its manL gene has been replaced by the mutated manL gene as defined in SEQ ID NO:111 (manL gene encoding the IIAB^{Man}₃₀₅ protein), called herein DGCC7710-IIAB^{Man}₃₀₅ strain;
b) carry out mutagenesis on the ccpA gene of the strain in a), for example by random or directed mutagenesis, to obtain a ccpA the sequence of which is different from the sequence of the ccpA gene of the DGCC7710-IIAB^{Man}₃₀₅ strain, to obtain a ccpA-mutated DGCC7710-IIAB^{Man}₃₀₅ strain;
c) determining by test B the ratio of the amount of galactose released (mM) over the amount of remaining lactose (mM) of a milk fermented by the ccpA-mutated DGCC7710-IIAB^{Man}₃₀₅ strain of step b), wherein a ratio of more than 1.2 means that the mutated ccpA is according to the invention. In a particular embodiment, a mutated ccpA gene is considered to be according to the invention when the ratio of the amount of galactose released (mM) over the amount of remaining lactose (mM) in the fermented milk is more than 1.5, is more than 2 or is more than 3.

Alternatively, the person skilled in the art can proceed by the following method:
a) provide a DGCC7710 strain in which its manM gene has been replaced by the mutated manM gene as defined in SEQ ID NO:157 (manM gene encoding the IIC^{Man}₂₀₈ protein), called herein DGCC7710-IIC^{Man}₂₀₈ strain;
b) carry out mutagenesis on the ccpA gene of the strain in a), for example by random or directed mutagenesis, to obtain a ccpA the sequence of which is different from the sequence of the ccpA gene of the DGCC7710-IIC^{Man}₂₀₈ strain, to obtain a ccpA-mutated DGCC7710-IIC^{Man}₂₀₈ strain;
c) determining by test B the ratio of the amount of galactose released (mM) over the amount of remaining lactose (mM) of a milk fermented by the ccpA-mutated DGCC7710-IIC^{Man}₂₀₈ strain of step b), wherein a ratio of more than 1.2 means that the mutated ccpA is according to the invention. In a particular embodiment, a mutated ccpA gene is considered to be according to the invention when the ratio of the amount of galactose released (mM) over the amount of remaining lactose (mM) in the fermented milk is more than 1.5, is more than 2 or is more than 3.

The person skilled in the art can also proceed by the following method:
a) provide a DGCC7710 strain in which its manN gene has been replaced by the mutated manN gene as defined in SEQ ID NO:206 (manN gene encoding the IID^{Man}₂₈ protein), called herein DGCC7710-IID^{Man}₂₈ strain;
b) carry out mutagenesis on the ccpA gene of the strain in a), for example by random or directed mutagenesis, to obtain a ccpA the sequence of which is different from the sequence of the ccpA gene of the DGCC7710-IID^{Man}₂₈ strain, to obtain a ccpA-mutated DGCC7710-IID^{Man}₂₈ strain;
c) determining by test B the ratio of the amount of galactose released (mM) over the amount of remaining lactose (mM) of a milk fermented by the ccpA-mutated DGCC7710-IID^{Man}₂₈ strain of step b), wherein a ratio of more than 1.2 means that the mutated ccpA is according to the invention. In a particular embodiment, a mutated ccpA gene is considered to be according to the invention when the ratio of the amount of galactose released (mM) over the amount of remaining lactose (mM) in the fermented milk is more than 1.5, is more than 2 or is more than 3.

Once identified, a mutated ccpA gene—as identified herein—can be introduced in lieu of the ccpA gene of a lactose-positive, galactose-negative, Streptococcus thermophilus strain, to obtain a lactose-positive, galactose-negative, Streptococcus thermophilus strain of the invention.

### Further Characterization of the Strains of the Invention

It is part of the invention that the lactose-positive, galactose-negative, Streptococcus thermophilus strain defined herein, when used to ferment milk by test B, exhibits a ratio of the amount of galactose released (mM) over the amount of remaining lactose (mM) which is more than 1.2, more than 1.5, more than 2 or more than 3. This feature can be used as described herein to design and identify strains of the invention. The inventors have shown that strains exhibiting this ratio (as a consequence of a mutation in a gene encoding a protein of the mannose-glucose-specific PTS and a mutation in the glcK gene and/or the ccpA gene) enable, when they are used to ferment milk:
1) to obtain a low lactose fermented milk; and/or
2) to obtain a fermented milk not undergoing post-acidification, even when stored at fermentation temperature.

Thus, if needed, these 2 advantages can be used to further characterize the strains of the invention, in addition to the ratio of the amount of galactose released (mM) over the amount of remaining lactose (mM) as assayed by test B, and the mutations defined herein for the gene encoding a protein of the mannose-glucose-specific PTS, the glcK gene and the ccpA gene.

In an embodiment, the lactose-positive, galactose-negative, Streptococcus thermophilus strain of the invention—in addition to exhibit, when used to ferment milk by test B, a ratio of the amount of galactose released (mM) over the amount of remaining lactose (mM) which is more than 1.2, more than 1.5, more than 2 or more than 3—is characterized by the fact that it leads to a low lactose fermented milk, when used to ferment milk by test B.

In an embodiment, the lactose-positive, galactose-negative, Streptococcus thermophilus strain of the invention—in addition to exhibit, when used to ferment milk by test B, a ratio of the amount of galactose released (mM) over the amount of remaining lactose (mM) which is more than 1.2, more than 1.5, more than 2 or more than 3—is characterized by the fact that it leads to a fermented milk, not undergoing post-acidification when stored at fermentation temperature.

In an embodiment, the lactose-positive, galactose-negative, Streptococcus thermophilus strain of the invention—in addition to exhibit, when used to ferment milk by test B, a ratio of the amount of galactose released (mM) over the amount of remaining lactose (mM) which is more than 1.2, more than 1.5, more than 2 or more than 3—is characterized by the fact that it leads to a low lactose fermented milk not undergoing post-acidification when stored at fermentation temperature (when used to ferment milk by test B).

The expression “low lactose fermented milk” means a fermented milk which has an amount of remaining lactose in the fermented milk, at the end of the fermentation by test B, which is less than 60 mM, less than 50 mM, less than 45 mM, less than 40 mM, less than 35 mM or less than 30 mM. In an embodiment, the concentration of remaining lactose in a fermented milk obtained by test B using a strain of the invention is less than 60 mM. In an embodiment, the concentration of remaining lactose in a fermented milk obtained by test B using a strain of the invention is less than 50 mM. In an embodiment, the concentration of remaining lactose in a fermented milk obtained by test B using a strain of the invention is less than 45 mM. In an embodiment, the concentration of remaining lactose in a fermented milk obtained by test B using a strain of the invention is less than 40 mM. In an embodiment, the concentration of remaining lactose in a fermented milk obtained by test B using a strain of the invention is less than 35 mM. In an embodiment, the concentration of remaining lactose in a fermented milk obtained by test B using a strain of the invention is less than 30 mM. In an embodiment, the concentration of remaining lactose in a fermented milk obtained by test B using a strain of the invention is selected from the group consisting of a concentration which is less than 60 mM, less than 50 mM, less than 45 mM, less than 40 mM, less than 35 mM and less than 30 mM. It is noteworthy that the amount of lactose in the milk provided in test B is before fermentation about 300 mM (60 g/L).

The expression “not undergoing post-acidification” means a milk product which, when inoculated with a strain of the invention and fermented by test B, has its pH decreased to a pH at which the speed of acidification definitively becomes less than 0.1 mUpH/min (defined herein as pH_{STOP}), wherein said pH_{STOP} is comprised between 4.6 and 5.3, and optionally the slope between pH6 and pH5.5 is at least −0.008 UpH/min.

Thus, the absence of post-acidification is characterized by the fact that the pH of the fermented milk stops between 4.6 and 5.3 when using test B. The pH is considered to be stopped (pH_{STOP}), when the speed of acidification (ΔpH/Δtime) definitively becomes less than 0.1 mUpH/min (less than 0.0001 UpH/min). By “definitively becomes”, it is meant that the speed of acidification stays less than 0.1 mUpH/min for the remaining time of the test B (i.e. up to 24h at fermentation temperature), once the pH_{STOP} is obtained.

In an embodiment, the pH_{STOP} obtained using a strain of the invention by test B is comprised between 4.7 and 5.2. In an embodiment, the pH_{STOP} obtained using a strain of the invention by test B is comprised between 4.8 and 5.1. In an embodiment, the pH_{STOP} obtained using a strain of the invention by test B is comprised between a minimal value selected from the group consisting of 4.6, 4.7 and 4.8 and a maximal value selected from the group consisting of 5.1, 5.2 and 5.3.

In an embodiment, the fermented milk not undergoing post-acidification is also characterized by the slope between pH6 and pH5.5. The slope represents the inverse of the velocity (speed of acidification). In an embodiment, the slope is at least −0.009 UpH/min. In an embodiment, the slope is at least −0.01 UpH/min.

### Examples of Some Strains of the Invention

The invention is also directed to the following lactose-positive, galactose-negative Streptococcus thermophilus strains:
a strain corresponding to the DSM32587 strain (deposited at the DSMZ on Aug. 15, 2017) into which the coding sequence of the manL gene is replaced by the sequence as set forth in SEQ ID NO:111 (encoding IIAB^{Man}₃₀₅) and its variants; a variant is defined herein as a lactose-positive, galactose-negative Streptococcus thermophilus strain bearing the same mutated manL gene, and which, when used to ferment milk as assayed by test B, the ratio of the amount of galactose released (mM) over the amount of remaining lactose (mM) in said fermented milk is more than 1.2;
a strain corresponding to the DSM32587 strain into which the coding sequence of the manM gene is replaced by the sequence as set forth in SEQ ID NO:157 (encoding IIC^{Man}₂₀₈) and its variants; a variant is defined herein as a lactose-positive, galactose-negative Streptococcus thermophilus strain bearing the same mutated manM gene, and which, when used to ferment milk as assayed by test B, the ratio of the amount of galactose released (mM) over the amount of remaining lactose (mM) in said fermented milk is more than 1.2;
a strain corresponding to the DSM32587 strain into which the coding sequence of the manN gene is replaced by the sequence as set forth in SEQ ID NO:206 (encoding IID^{Man}₂₈) and its variants; a variant is defined herein as a lactose-positive, galactose-negative Streptococcus thermophilus strain bearing the same mutated manN gene, and which, when used to ferment milk as assayed by test B, the ratio of the amount of galactose released (mM) over the amount of remaining lactose (mM) in said fermented milk is more than 1.2;
a strain corresponding to the DSM32587 strain into which the coding sequence of the ccpA gene is replaced by the sequence as set forth in SEQ ID NO:71 (ccpA_{A}A114-120) and the coding sequence of the manL gene is replaced by the sequence as set forth in SEQ ID NO: 11 (encoding IIAB^{Man}₃₀₅) and its variants; a variant is defined herein as a lactose-positive, galactose-negative Streptococcus thermophilus strain bearing the same mutated ccpA gene and same mutated manL gene, and which, when used to ferment milk as assayed by test B, the ratio of the amount of galactose released (mM) over the amount of remaining lactose (mM) in said fermented milk is more than 1.2;
a strain corresponding to the DSM32587 strain into which the coding sequence of the ccpA gene is replaced by the sequence as set forth in SEQ ID NO:71 (ccpA_{A}A114-120) and the coding sequence of the manM gene is replaced by the sequence as set forth in SEQ ID NO:157 (encoding IIC^{Man}₂₀₈) and its variants; a variant is defined herein as a lactose-positive, galactose-negative Streptococcus thermophilus strain bearing the same mutated ccpA gene and same mutated manM gene, and which, when used to ferment milk as assayed by test B, the ratio of the amount of galactose released (mM) over the amount of remaining lactose (mM) in said fermented milk is more than 1.2;
a strain corresponding to the DSM32587 strain into which the coding sequence of the ccpA gene is replaced by the sequence as set forth in SEQ ID NO:71 (ccpA_{Δ1A114-120}) and the coding sequence of the manN gene is replaced by the sequence as set forth in SEQ ID NO:206 (encoding IID^{Man}₂₈) and its variants; a variant is defined herein as a lactose-positive, galactose-negative Streptococcus thermophilus strain bearing the same mutated ccpA gene and same mutated manN gene, and which, when used to ferment milk as assayed by test B, the ratio of the amount of galactose released (mM) over the amount of remaining lactose (mM) in said fermented milk is more than 1.2;
a strain corresponding to the DSM28255 strain into which the coding sequence of the ccpA gene is replaced by the sequence as set forth in SEQ ID NO:71 (ccpA_{Δ1A114-120}) and the coding sequence of the manL gene is replaced by the sequence as set forth in SEQ ID NO:111 (encoding IIAB^{Man}₃₀₅) and its variants; a variant is defined herein as a lactose-positive, galactose-negative Streptococcus thermophilus strain bearing the same mutated ccpA gene and same mutated manL gene, and which, when used to ferment milk as assayed by test B, the ratio of the amount of galactose released (mM) over the amount of remaining lactose (mM) in said fermented milk is more than 1.2.
a strain corresponding to the DSM28255 strain into which the coding sequence of the ccpA gene is replaced by the sequence as set forth in SEQ ID NO:71 (ccpA_{Δ1A114-120}) and the coding sequence of the manM gene is replaced by the sequence as set forth in SEQ ID NO:157 (encoding IIC^{Man}₂₀₈) and its variants; a variant is defined herein as a lactose-positive, galactose-negative Streptococcus thermophilus strain bearing the same mutated ccpA gene and same mutated manM gene, and which, when used to ferment milk as assayed by test B, the ratio of the amount of galactose released (mM) over the amount of remaining lactose (mM) in said fermented milk is more than 1.2;
a strain corresponding to the DSM28255 strain into which the coding sequence of the ccpA gene is replaced by the sequence as set forth in SEQ ID NO:71 (ccpA_{Δ1A114-120}) and the coding sequence of the manN gene is replaced by the sequence as set forth in SEQ ID NO:206 (encoding IID^{Man}₂₈) and its variants; a variant is defined herein as a lactose-positive, galactose-negative Streptococcus thermophilus strain bearing the same mutated ccpA gene and same mutated manN gene, and which, when used to ferment milk as assayed by test B, the ratio of the amount of galactose released (mM) over the amount of remaining lactose (mM) in said fermented milk is more than 1.2.

In a particular embodiment, the genome sequence of the strain variant as defined herein has an identity of at least 90%, with the genome sequence of the strain the variant is obtained from, in particular an identity of at least 90%, at least 91%, at least 95%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.94%, at least 99.96%, at least 99.98%, or at least 99.99% with the genome sequence of the strain the variant is obtained from. The identity is described in comparing the two genome sequences over their full-length (global alignment), and may be calculated using any program based on the Needleman-Wunsch algorithm.

Composition, Method and Use with Strains the Lactose-Positive, Galactose-Negative Streptococcus thermophilus Strains of the Invention

The invention is also directed to a bacterial composition comprising or consisting of at least one, in particular one, lactose-positive, galactose-negative Streptococcus thermophilus strain of the invention. In a particular embodiment, the bacterial composition is a pure culture, i.e., comprises or consists of a single bacterium strain. In another embodiment, the bacterial composition is a mixed culture, i.e., comprises or consists of the lactose-positive, galactose-negative Streptococcus thermophilus strain(s) of the invention and at least one other bacterium strain. By “at least” (in reference to a strain or bacterium), it is meant 1 or more, and in particular 1, 2, 3, 4 or 5 strains.

Thus, in an embodiment, a bacterial composition of the invention comprises or consists of the lactose-positive, galactose-negative Streptococcus thermophilus strain(s) of the invention and at least one lactic acid bacterium of the species selected from the group consisting of a Lactococcus species, a Streptococcus species, a Lactobacillus species including Lactobacillus acidophilus, an Enterococcus species, a Pediococcus species, a Leuconostoc species, a Bifidobacterium species and an Oenococcus species or any combination thereof. Lactococcus species include Lactobacillus acidophilus and Lactococcus lactis, including Lactococcus lactis subsp. lactis, Lactococcus lactis subsp. cremoris and Lactococcus lactis subsp. lactis biovar diacetylactis. Bifidobacterium species includes Bifidobacterium animalis, in particular Bifidobacterium animalis subsp lactis. Other lactic acid bacteria species include Leuconostoc sp., Streptococcus thermophilus, Lactobacillus delbrueckii subsp. bulgaricus, and Lactobacillus helveticus.

In an embodiment, the bacterial composition comprises or consists of the lactose-positive, galactose-negative Streptococcus thermophilus strain(s) of the invention, and at least one Streptococcus thermophilus strain, different from the S. thermophilus strain(s) of the invention and/or at least one strain of the Lactobacillus species, and/or any combination thereof. In a particular embodiment, the bacterial composition comprises or consists of the Streptococcus thermophilus strain(s) of the invention, one or several strain(s) of the species Lactobacillus delbrueckii subsp. bulgaricus and/or one or several strain(s) of the species Lactobacillus helveticus and/or any combination thereof, and optionally at least one Streptococcus thermophilus strain, different from the S. thermophilus strain(s) of the invention. In a particular embodiment, the bacterial composition comprises or consists of the Streptococcus thermophilus strain(s) of the invention, at least one strain of species Streptococcus thermophilus, different from the S. thermophilus strain(s) of the invention, and a strain of the species Lactobacillus delbrueckii subsp. bulgaricus. In another particular embodiment, the bacterial composition comprises or consists of the Streptococcus thermophilus strain(s) of the invention, and a strain of the species Lactobacillus delbrueckii subsp. bulgaricus.

In an embodiment, the bacterial composition comprises or consists of the Streptococcus thermophilus strain(s) of the invention, a Lactococcus lactis subsp. lactis and/or a Lactococcus lactis subsp. cremoris.

In a particular embodiment of any bacterial composition defined herein, either as a pure or mixed culture, the bacterial composition further comprises at least one probiotic strain such as Bifidobacterium animalis subsp. lactis, Lactobacillus acidophilus, Lactobacillus paracasei, or Lactobacillus casei.

In a particular embodiment, the bacterial composition, either as a pure or mixed culture as defined above is under frozen, dried, freeze-dried, liquid or solid format, in the form of pellets or frozen pellets, or in a powder or dried powder. In a particular embodiment, the bacterial composition of the invention is in a frozen format or in the form of pellets or frozen pellets, in particular contained into one or more box or sachet. In another embodiment, the bacterial composition as defined herein is under a powder form, such as a dried or freeze-dried powder, in particular contained into one or more box or sachet.

In a particular embodiment, the bacterial composition of the invention, either as a pure culture or mixed culture as defined above, and whatever the format (frozen, dried, freeze-dried, liquid or solid format, in the form of pellets or frozen pellets, or in a powder or dried powder) comprises the lactose-positive, galactose-negative Streptococcus thermophilus strain(s) of the invention in a concentration comprised in the range of 10⁵ to 10¹² cfu (colony forming units) per gram of the bacterial composition. In a particular embodiment, the concentration of the lactose-positive, galactose-negative Streptococcus thermophilus strain(s) within the bacterial composition of the invention is in the range of 10⁷ to 10¹² cfu per gram of the bacterial composition, and in particular at least 10⁷, at least 10⁸, at least 10⁹, at least 10¹⁰ or at least 10¹¹ CFU/g of the bacterial composition. In a particular embodiment, when in the form of frozen or dried concentrate, the concentration of the lactose-positive, galactose-negative Streptococcus thermophilus strain(s)—as pure culture or as a mixed culture—within the bacterial composition is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

The invention also concerns a method for manufacturing a fermented product, comprising a) inoculating a substrate with the lactose-positive, galactose-negative Streptococcus thermophilus strain(s) of the invention and b) fermenting said inoculated substrate, to obtain a fermented product. In a particular embodiment, the lactose-positive, galactose-negative Streptococcus thermophilus strain(s) of the invention is inoculated as a bacterial composition as defined herein, such as a pure culture or a mixed culture. In an embodiment, the substrate into which the S. thermophilus strain(s) or bacterial composition of the invention is added to is milk substrate. By “milk substrate”, it is meant milk of animal and/or plant origin. In a particular embodiment, the milk substrate is of animal origin, such as cow, goat, sheep, buffalo, zebra, horse, donkey, or camel, and the like. The milk may be in the native state, a reconstituted milk, a skimmed milk, or a milk supplemented with compounds necessary for the growth of the bacteria or for the subsequent processing of fermented milk. Therefore, in a particular embodiment, the invention also provides a method for manufacturing a fermented dairy product, comprising a) inoculating a milk substrate with the lactose-positive, galactose-negative Streptococcus thermophilus strain(s) or bacterial composition of the invention and b) fermenting said inoculated milk substrate, to obtain a fermented dairy product.

The invention is also directed to the use of the lactose-positive, galactose-negative, Streptococcus thermophilus strain(s) of the invention or a composition of the invention, to manufacture a fermented dairy product.

The invention is also directed to a fermented dairy product, which is obtained using the lactose-positive, galactose-negative Streptococcus thermophilus strain(s) of the invention or a bacterial composition of the invention, in particular obtained or obtainable by the method of the invention. Thus, the invention is directed to a fermented dairy product comprising the lactose-positive, galactose-negative Streptococcus thermophilus strain(s) of the invention. In a particular embodiment, the fermented dairy food product of the invention is fresh fermented milk. In a particular embodiment, the fermented dairy product of the invention—in particular the fresh fermented milk as defined herein—contains the DSM32587 strain deposited at the DSMZ on Aug. 15, 2017 or any variant thereof as defined herein.

Proteins, Nucleic Acids, Vectors, Constructs and their Uses

The invention is directed to a polynucleotide encoding a Streptococcus thermophilus glucokinase, the glucokinase activity of which is significantly reduced but not null. In an embodiment, the reduced but not null glucokinase activity is determined in a DGCC7710 derivative, i.e., a DGCC7710 strain into which its glcK gene has been replaced by the glcK polynucleotide to be assayed. To test that a polynucleotide encoding a glucokinase fulfils the “significantly reduced but not null” glucokinase activity feature in a DGCC7710 derivative, the glcK gene of the DGCC7710 strain is replaced by the glcK gene encoding the Streptococcus thermophilus glucokinase to be assayed to obtain the derivative of DGCC7710, and the DGCC7710 derivative is assayed by test A (see example 4).

A Streptococcus thermophilus glucokinase (encoded by a polynucleotide of the invention) fulfils the “significantly reduced but not null” glucokinase activity feature in a DGCC7710 derivative, when:
a) either the glucokinase activity of said Streptococcus thermophilus glucokinase in a DGCC7710 derivative is between 200 and 1500 U/g of total protein extract, as assayed by test A, in particular between 300 and 1200 U/g or between 400 and 1000 U/g of total protein extract, as assayed by test A, or
b) the glucokinase activity of said Streptococcus thermophilus glucokinase in a DGCC7710 derivative is between 5 and 60% the glucokinase activity of the DGCC7710 strain deposited at the DSMZ under accession number DSM28255 on Jan. 14, 2014, when both assayed by test A, in particular between 10 and 50% or between 15 and 40% the glucokinase activity of the DGCC7710 strain, wherein the glucokinase activity in said DG7710 derivative and the glucokinase activity of the DGCC7710 strain are assayed by test A.

In a particular embodiment, the glucokinase activity of the Streptococcus thermophilus glucokinase in a DGCC7710 derivative is between 200 and 1500 U/g of total protein extract, as assayed by test A. In a particular embodiment, the glucokinase activity of the Streptococcus thermophilus glucokinase in a DGCC7710 derivative is between 300 and 1200 U/g of total protein extract, as assayed by test A. In a particular embodiment, the glucokinase activity of the Streptococcus thermophilus glucokinase in a DGCC7710 derivative is between 400 and 1000 U/g of total protein extract, as assayed by test A. In a particular embodiment, the glucokinase activity of the Streptococcus thermophilus glucokinase in a DGCC7710 derivative is between a minimal value selected from the group consisting of 200, 300 and 400 U/g of total protein extract and a maximal value selected from the group consisting of 1000, 1200 and 1500 U/g of total protein extract, as assayed by test A. It is noteworthy that, as mentioned in test A, the glucokinase activity values disclosed herein are the mean of three experiments (triplicates).

In a particular embodiment, the glucokinase activity of the Streptococcus thermophilus glucokinase (encoded by a polynucleotide of the invention) in a DGCC7710 derivative is between 5 and 60% the activity of the glucokinase activity of the DGCC7710 strain deposited at the DSMZ under accession number DSM28255 on Jan. 14, 2014. By “glucokinase activity of the DGCC7710 strain” it is meant the activity of the DGCC7710 strain glucokinase (i.e., with SEQ ID NO:2) as assayed by test A in the DGCC7710 strain [i.e., the test A is carried out using the DGCC7710 strain]. The percentage value is calculated based on the glucokinase activity of the Streptococcus thermophilus glucokinase in a DGCC7710 derivative and the glucokinase activity of the DGCC7710 strain, both assayed by test A.

In a particular embodiment, the glucokinase activity of the Streptococcus thermophilus glucokinase in a DGCC7710 derivative is between 5 and 60% the glucokinase activity of the DGCC7710 strain. In a particular embodiment, the glucokinase activity of the Streptococcus thermophilus glucokinase in a DGCC7710 derivative is between 10 and 50% the glucokinase activity of the DGCC7710 strain. In a particular embodiment, the glucokinase activity of the Streptococcus thermophilus glucokinase in a DGCC7710 derivative is between 15 and 40% the glucokinase activity of the strain DGCC7710. In a particular embodiment, the glucokinase activity of the Streptococcus thermophilus glucokinase in a DGCC7710 derivative is between a minimal percentage selected from the group consisting of 5, 10 and 15% the glucokinase activity of the DGCC7710 strain and a maximal percentage selected from the group consisting of 40, 50 and 60% the glucokinase activity of the DGCC7710 strain. In a particular embodiment and whatever the range of percentages, the glucokinase activity of the Streptococcus thermophilus glucokinase is assayed in a DGCC7710 derivative by test A as described herein. It is noteworthy that the percentage values disclosed herein are calculated based on glucokinase activity values which are the mean of three independent experiments (triplicates) as assayed by test A.

The feature “glucokinase activity in a DGCC7710 derivative is significantly reduced but not null” can also be characterized, in a DGCC7710 derivative, by the maximum forward velocity of the glucokinase (Vmax) or by the affinity of the glucokinase (called Km) for one or two of its substrates, i.e., glucose and ATP. In an embodiment, the feature “significantly reduced but not null glucokinase activity in a DGCC7710 derivative” of the Streptococcus thermophilus glucokinase (encoded by a polynucleotide of the invention) is further characterized by the maximum forward velocity of this glucokinase in a DGCC7710 derivative.

Therefore, in combination with the embodiment of the feature “glucokinase activity in a DGCC7710 derivative is significantly reduced but not null” defined herein, the maximum forward velocity (Vmax) of the Streptococcus thermophilus glucokinase is significantly reduced but not null in a DGCC7710 derivative. To test that a glucokinase fulfils the “significantly reduced but not null” Vmax feature in a DGCC7710 derivative, the open reading frame of the glcK gene of the DGCC7710 strain is replaced by the open reading frame of the glcK gene encoding the Streptococcus thermophilus glucokinase to be assayed (i.e., the polynucleotide of the invention) to obtain a derivative of DGCC7710, and the DGCC7710 derivative is assayed by test C (see example 4). The expression “DGCC7710 derivative” is as defined above.

The “significantly reduced but not null in a DGCC7710 derivative” feature of the Vmax of the glucokinase can be defined by one or two of these parameters:
the Vmax of the Streptococcus thermophilus glucokinase in a DGCC7710 derivative is between 200 and 1500 U/g total protein extract, as assayed by test C;
the Vmax of the Streptococcus thermophilus glucokinase in a DGCC7710 derivative is between 5 and 60% the Vmax of the glucokinase of the DGCC7710 strain deposited at the DSMZ under accession number DSM28255 on Jan. 14, 2014, when both assayed by test C.

In a particular embodiment, the invention relates to a polynucleotide encoding a Streptococcus thermophilus glucokinase, the glucokinase activity of which in a DGCC7710 derivative is significantly reduced but not null (as defined herein) and wherein the maximum forward velocity (Vmax) of said glucokinase in a DGCC7710 derivative is significantly reduced but not null, and defined by one or two of these parameters:
the Vmax of the Streptococcus thermophilus glucokinase in a DGCC7710 derivative is between 200 and 1500 U/g total protein extract, as assayed by test C;
the Vmax of the Streptococcus thermophilus glucokinase in a DGCC7710 derivative is between 5 and 60% the Vmax of the glucokinase of the DGCC7710 strain deposited at the DSMZ under accession number DSM28255 on Jan. 14, 2014, when both assayed by test C.

In a particular embodiment, the Vmax of the Streptococcus thermophilus glucokinase in a DGCC7710 derivative is between 200 and 1500 U/g total protein extract, as assayed by test C. In a particular embodiment, the Vmax of the Streptococcus thermophilus glucokinase in a DGCC7710 derivative is between 300 and 1200 U/g total protein extract, as assayed by test C. In a particular embodiment, the Vmax of the Streptococcus thermophilus glucokinase in a DGCC7710 derivative is between 400 and 1000 U/g total protein extract. In a particular embodiment, the Vmax of the Streptococcus thermophilus glucokinase in a DGCC7710 derivative is between a minimal value selected from the group consisting of 200, 300 and 400 U/g of total protein extract and a maximal value selected from the group consisting of 1000, 1200 and 1500 U/g of total protein extract, as assayed by test C.

In a particular embodiment, the Vmax of the Streptococcus thermophilus glucokinase in a DGCC7710 derivative is between 10 and 50% the Vmax of the glucokinase of the DGCC7710 strain, when both assayed by test C. By “Vmax of the glucokinase of the DGCC7710 strain” it is meant the Vmax of the DGCC7710 strain glucokinase (i.e., with SEQ ID NO:2) as assayed by test C in the DGCC7710 strain [i.e., the test C is carried out using the DGCC7710 strain]. The percentage value is calculated based on the Vmax of the Streptococcus thermophilus glucokinase in a DGCC7710 derivative and the Vmax of the glucokinase of DGCC7710 strain, both assayed by test C. In a particular embodiment, the Vmax of the Streptococcus thermophilus glucokinase in a DGCC7710 derivative is between 15 and 40% the Vmax of the glucokinase of the DGCC7710 strain. In a particular embodiment, the Vmax of the Streptococcus thermophilus glucokinase in a DGCC7710 derivative is between a minimal percentage selected from the group consisting of 5, 10 and 15% the Vmax of the glucokinase activity of the DGCC7710 strain and a maximal percentage selected from the group consisting of 40, 50 and 60% the Vmax of the glucokinase activity of the DGCC7710 strain.

In an embodiment, the polynucleotide of the invention encodes a Streptococcus thermophilus glucokinase having a significantly reduced but not null glucokinase activity in a DGCC7710 derivative and optionally a significantly reduced but not null Vmax in a DGCC7710 derivative, the sequence of which has at its position 275 (numbering based on the sequence as defined in SEQ ID NO:25) an amino acid which is not a glutamic acid (i.e., is any amino acid except a glutamic acid). In an embodiment, the polynucleotide of the invention encodes a Streptococcus thermophilus glucokinase having a significantly reduced but not null glucokinase activity in a DGCC7710 derivative and optionally a significantly reduced but not null in a DGCC7710 derivative, the sequence of which has at its position 275 (numbering based on the sequence as defined in SEQ ID NO:25) an amino acid which is not an acidic amino acid (i.e., is any amino acid except an acidic amino acid). In an embodiment, the polynucleotide of the invention encodes a Streptococcus thermophilus glucokinase having a significantly reduced but not null glucokinase activity in a DGCC7710 derivative and optionally a significantly reduced but not null Vmax in a DGCC7710 derivative, the sequence of which has at its position 275 (numbering based on the sequence as defined in SEQ ID NO:25) an amino acid which is selected from the group consisting of a lysine and any of its conservative amino acids. In an embodiment, the polynucleotide of the invention encodes a Streptococcus thermophilus glucokinase having a significantly reduced but not null glucokinase activity in a DGCC7710 derivative and optionally a significantly reduced but not null in a DGCC7710 derivative, the sequence of which has at its position 275 (numbering based on the sequence as defined in SEQ ID NO:25) an amino acid which is a lysine. In a particular embodiment, the polynucleotide of the invention encodes a Streptococcus thermophilus which is 322 amino acids in length.

In another embodiment, the polynucleotide of the invention encodes a Streptococcus thermophilus glucokinase having a significantly reduced but not null glucokinase activity in a DGCC7710 derivative and optionally a significantly reduced but not null Vmax in a DGCC7710 derivative, the sequence of which has at its position 144 (numbering based on the sequence as defined in SEQ ID NO:25) an amino acid which is not a glycine (i.e., is any amino acid except a glycine). In an embodiment, the polynucleotide of the invention encodes a Streptococcus thermophilus glucokinase having a significantly reduced but not null glucokinase activity in a DGCC7710 derivative and optionally a significantly reduced but not null in a DGCC7710 derivative, the sequence of which has at its position 144 (numbering based on the sequence as defined in SEQ ID NO:25) an amino acid which is not an aliphatic amino acid (i.e., is any amino acid except an aliphatic amino acid). In an embodiment, the polynucleotide of the invention encodes a Streptococcus thermophilus glucokinase having a significantly reduced but not null glucokinase activity in a DGCC7710 derivative and optionally a significantly reduced but not null Vmax in a DGCC7710 derivative, the sequence of which has at its position 144 (numbering based on the sequence as defined in SEQ ID NO:25) an amino acid which is selected from the group consisting of a serine and any of its conservative amino acids. In an embodiment, the polynucleotide of the invention encodes a Streptococcus thermophilus glucokinase having a significantly reduced but not null glucokinase activity in a DGCC7710 derivative and optionally a significantly reduced but not null in a DGCC7710 derivative, the sequence of which has at its position 144 (numbering based on the sequence as defined in SEQ ID NO:25) an amino acid which is a serine. In a particular embodiment, the polynucleotide of the invention encodes a Streptococcus thermophilus glucokinase which is 322 amino acids in length.

In a particular embodiment, the polynucleotide of the invention encodes a Streptococcus thermophilus glucokinase having a significantly reduced but not null glucokinase activity in a DGCC7710 derivative and optionally a significantly reduced but not null in a DGCC7710 derivative is selected from the group consisting of:
a) a sequence as defined in SEQ ID NO:25, wherein the amino acid at position 275 is any amino acid except a glutamic acid, in particular is any amino acid except an acidic amino acid, in particular is a lysine; and
b) a GlcK variant sequence having at least 90% similarity or identity with SEQ ID NO:25, wherein the amino acid of the glucokinase corresponding to position 275 of SEQ ID NO:25 (or the amino acid at position 275 of the glucokinase) is any amino acid except a glutamic acid, in particular is any amino acid except an acidic amino acid, in particular is a lysine. In a particular embodiment, the GlcK variant sequence is 322-amino acids in length. In an embodiment, said GlcK variant has an arginine at its position 278 and/or a serine at its position 279.
c) a sequence as defined in SEQ ID NO:46, wherein the amino acid at position 144 is any amino acid except a glycine, in particular is any amino acid except an aliphatic amino acid, in particular is a serine; and
d) a GlcK variant sequence having at least 90% similarity or identity with SEQ ID NO:46, wherein the amino acid of the glucokinase corresponding to position 144 of SEQ ID NO:46 (or the amino acid at position 144 of the glucokinase) is any amino acid except a glycine, in particular is any amino acid except an aliphatic amino acid, in particular is a serine. In a particular embodiment, the GlcK variant sequence is 322-amino acids in length.

For the definition of the GlcK variant having at least 90% similarity or identity with SEQ ID NO:25, the similarity or identity is calculated herein over the whole length of the 2 sequences after optimal alignment [i.e., number of similar or identical amino acid residues in the aligned parts(s) of the sequences]; the position 275 as defined in SEQ ID NO:25 is not considered for the calculation of the similarity or identity. In a particular embodiment, the GlcK variant sequence has at least 91, 92, 93, 94, 95, 96, 97, 98 or 99% similarity or identity with SEQ ID NO:25, wherein the amino acid of the glucokinase corresponding to position 275 of SEQ ID NO:25 (or the amino acid at position 275 of the glucokinase) is any amino acid except a glutamic acid, in particular is any amino acid except an acidic amino acid, in particular is a lysine. In an embodiment, the GlcK variant sequence has at least 95% similarity or identity with SEQ ID NO:25, wherein the amino acid corresponding to position 275 of SEQ ID NO:25 (or the amino acid at position 275 of the glucokinase) is any amino acid except a glutamic acid, in particular is any amino acid except an acidic amino acid, in particular is a lysine. In an embodiment, the GlcK variant sequence has at least 97% similarity or identity with SEQ ID NO:25, wherein the amino acid corresponding to position 275 of SEQ ID NO:25 (or the amino acid at position 275 of the glucokinase) is any amino acid except a glutamic acid, in particular is any amino acid except an acidic amino acid, in particular is a lysine.

In a particular embodiment, the GlcK variant sequence differs from SEQ ID NO:25 by from 1 to 30 amino acid substitutions wherein the amino acid at position 275 of said GlcK variant is any amino acid except a glutamic acid, in particular is any amino acid except an acidic amino acid, in particular is a lysine (the position 275 is not considered for the calculation of the number of substitution(s)). In a particular embodiment, the GlcK variant sequence differs from SEQ ID NO:25 by from 1 to 20 amino acid substitutions, wherein the amino acid at position 275 of said GlcK variant is any amino acid except a glutamic acid, in particular is any amino acid except an acidic amino acid, in particular is a lysine. In a particular embodiment, the GlcK variant sequence differs from SEQ ID NO:25 by from 1 to 15 amino acid substitutions wherein the amino acid at position 275 of said GlcK variant is any amino acid except a glutamic acid, in particular is any amino acid except an acidic amino acid, in particular is a lysine. In a particular embodiment, the GlcK sequence differs from SEQ ID NO:25 by from 1 to 10 amino acid substitutions wherein the amino acid at position 275 of said GlcK variant is any amino acid except a glutamic acid, in particular is any amino acid except an acidic amino acid, in particular is a lysine. In a particular embodiment, the GlcK sequence differs from SEQ ID NO:25 by 1, 2, 3, 4, 5, 6, 7, 8, 9, 10, 11 or 12 amino acid substitutions wherein the amino acid at position 275 of said GlcK variant is any amino acid except a glutamic acid, in particular is any amino acid except an acidic amino acid, in particular is a lysine.

In a particular embodiment, the sequence of the Streptococcus thermophilus glucokinase (encoded by a polynucleotide of the invention) is selected from the group consisting of SEQ ID NOs: 25, 26, 27, 28, 29, 30, 31, 32, 33 and 34, wherein the amino acid at position 275 of said variant is any amino acid except a glutamic acid, in particular is any amino acid except an acidic amino acid, in particular is a lysine. In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is not a glutamic acid.

In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase (encoded by a polynucleotide of the invention) corresponding to position 275 of SEQ ID NO:25 (or the amino acid at position 275 of the glucokinase) is not an acidic amino acid. In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is selected from the group consisting of a lysine and any of its conservative amino acids. In a particular embodiment, either as the SEQ ID NO:25 or any GlcK variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is a lysine; thus, in a particular embodiment, the sequence of the Streptococcus thermophilus glucokinase (encoded by a polynucleotide of the invention) is selected from the group consisting of SEQ ID NOs: 22, 35, 36, 37, 38, 39, 40, 41, 42 and 43.

For the definition of the GlcK variant having at least 90% similarity or identity with SEQ ID NO:46, the similarity or identity is calculated herein over the whole length of the 2 sequences after optimal alignment [i.e., number of similar or identical amino acid residues in the aligned parts(s) of the sequences]; the position 144 as defined in SEQ ID NO:46 is not considered for the calculation of the similarity or identity. In a particular embodiment, the GlcK variant sequence has at least 91, 92, 93, 94, 95, 96, 97, 98 or 99% similarity or identity with SEQ ID NO:46, wherein the amino acid of the glucokinase corresponding to position 144 of SEQ ID NO:46 (or the amino acid at position 144 of the glucokinase) is any amino acid except a glycine, in particular is any amino acid except an aliphatic amino acid, in particular is a serine. In an embodiment, the GlcK variant sequence has at least 95% similarity or identity with SEQ ID NO:46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine. In an embodiment, the GlcK variant sequence has at least 97% similarity or identity with SEQ ID NO:46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine.

In a particular embodiment, the GlcK variant sequence differs from SEQ ID NO:46 by from 1 to 30 amino acid substitutions wherein the amino acid at position 144 of said GlcK variant is any amino acid except a glycine, in particular is any amino acid except an aliphatic amino acid, in particular is a serine (the position 144 is not considered for the calculation of the number of substitution(s)). In a particular embodiment, the GlcK variant sequence differs from SEQ ID NO:46 by from 1 to 20 amino acid substitutions, wherein the amino acid at position 144 of said GlcK variant is any amino acid except a glycine, in particular is any amino acid except an aliphatic amino acid, in particular is a serine. In a particular embodiment, the GlcK variant sequence differs from SEQ ID NO:46 by from 1 to 15 amino acid substitutions wherein the amino acid at position 144 of said GlcK variant is any amino acid except a glycine, in particular is any amino acid except an aliphatic amino acid, in particular is a serine. In a particular embodiment, the GlcK sequence differs from SEQ ID NO:46 by from 1 to 10 amino acid substitutions wherein the amino acid at position 144 of said GlcK variant is any amino acid except a glycine, in particular is any amino acid except an aliphatic amino acid, in particular is a serine. In a particular embodiment, the GlcK sequence differs from SEQ ID NO:46 by 1, 2, 3, 4, 5, 6, 7, 8, 9, 10, 11 or 12 amino acid substitutions wherein the amino acid at position 144 of said GlcK variant is any amino acid except a glycine, in particular is any amino acid except an aliphatic amino acid, in particular is a serine.

In a particular embodiment, the sequence of the Streptococcus thermophilus glucokinase (encoded by a polynucleotide of the invention) is selected from the group consisting of SEQ ID NOs: 46, 47, 48, 49, 50, 51, 52, 53, 54 and 55, wherein the amino acid at position 144 of said variant is any amino acid except a glycine, in particular is any amino acid except an aliphatic amino acid, in particular is a serine. In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is not a glycine.

In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase (encoded by a polynucleotide of the invention) corresponding to position 144 of SEQ ID NO:46 (or the amino acid at position 144 of the glucokinase) is not an aliphatic amino acid. In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is selected from the group consisting of a serine and any of its conservative amino acids. In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is a serine; thus, in a particular embodiment, the sequence of the Streptococcus thermophilus glucokinase (encoded by a polynucleotide of the invention) is selected from the group consisting of SEQ ID NOs: 45, 56, 57, 58, 59, 60, 61, 62, 63 and 64.

When defining the sequence of the Streptococcus thermophilus glucokinase (encoded by a polynucleotide of the invention), it is according to the teaching of this application that the glucokinase activity in a DGCC7710 derivative expressing this glucokinase is significantly reduced but not null as defined herein and optionally that the Vmax of this glucokinase in a DGCC7710 derivative is significantly reduced but not null as defined herein.

The invention is also directed to a polynucleotide encoding a322-amino acid glucokinase. In a particular embodiment, said polynucleotide is from a Streptococcus thermophilus strain. Based on the genetic code, the person skilled in the art knows whether a polynucleotide encodes a Streptococcus thermophilus glucokinase as defined herein. In a particular embodiment, when the encoded glucokinase is 322 amino acids in length, the polynucleotide of the invention is 969 nucleotides in length.

A non-limitative example of a polynucleotide of the invention is disclosed in SEQ ID NO:21. Another non-limitative example of a polynucleotide of the invention is disclosed in SEQ ID NO:44. Other non-limitative examples of polynucleotides of the invention are the sequences as defined in SEQ ID Nos: 1, 3, 5, 7, 9, 11, 13, 15, 17 and 19, but into which the codon 144 or 275 encodes any amino acid except a glycine or glutamic acid respectively, in particular encodes any amino acid except an aliphatic or acidic amino acid respectively, in particular encodes a serine or lysine respectively. In particular, non-limitative examples of polynucleotides of the invention are the sequences as defined in SEQ ID Nos: 1, 3, 5, 7, 9, 11, 13, 15, 17 and 19, but into which the codon 275 is AAA or AAG. In particular, non-limitative examples of polynucleotides of the invention are the sequences as defined in SEQ ID Nos: 1, 3, 5, 7, 9, 11, 13, 15, 17 and 19, but into which the codon 144 is AGT, AGC, TCT, TCC, TCA or TCG.

The invention also concerns the use a polynucleotide of the invention (or a construct, plasmid or vector) to design a bacterial cell, in particular a gram-positive bacterial cell, in particular a Streptococcus thermophilus cell. In a particular embodiment, the polynucleotide of the invention (or a construct, plasmid or vector) is used to replace the glcK gene of a Streptococcus thermophilus strain, such that the Streptococcus thermophilus strain expresses a glucokinase as defined herein. In a particular embodiment, the only glucokinase expressed by said obtained Streptococcus thermophilus is a glucokinase as defined herein. In a particular embodiment, the polynucleotide of the invention (or a construct, plasmid or vector) is used to replace the glcK gene of a lactose-positive Streptococcus thermophilus strain. In an embodiment, the polynucleotide of the invention (or a construct, plasmid or vector) is used to replace the glcK gene of a lactose-positive, galactose-negative Streptococcus thermophilus strain.

The invention is also directed to a (mutated) Streptococcus thermophilus ccpA polynucleotide, as defined or as identified above. In an embodiment, the invention is directed to a (mutated) Streptococcus thermophilus ccpA polynucleotide selected from the group consisting of:
a) a ccpA polynucleotide, which when inserted in lieu of the ccpA gene of the DGCC7710 strain, leads to a DGCC7710 derivative exhibiting a ratio of beta-galactosidase activity as assayed by test D over the glucokinase activity as assayed by test E of at least 4·10⁻⁶ as defined herein.
b) a ccpA polynucleotide, which when inserted in lieu of the ccpA gene of a DGCC7710 strain into which the manL gene has previously been replaced by a manL gene as defined in SEQ ID NO:111 (i.e., of the DGCC7710-IIAB^{Man}₃₀₅ strain) leads to a DGCC7710-IIAB^{Man}₃₀₅ derivative, which, when used to ferment milk as assayed by test B, exhibits a ratio of the amount of galactose released (mM) over the amount of remaining lactose (mM) in said fermented milk which is more than 1.2, more than 1.5, more than 2, more than 2.5 or more than 3
c) a ccpA polynucleotide, which when inserted in lieu of the ccpA gene of a DGCC7710 strain into which the manM gene has previously been replaced by a manM gene as defined in SEQ ID NO:157 (i.e., of the DGCC7710-IIC^{Man}₂₀₈ strain) leads to a DGCC7710-IIC^{Man}₂₀₈ derivative, which, when used to ferment milk as assayed by test B, exhibits a ratio of the amount of galactose released (mM) over the amount of remaining lactose (mM) in said fermented milk which is more than 1.2, more than 1.5, more than 2, more than 2.5 or more than 3;
d) a ccpA polynucleotide, which when inserted in lieu of the ccpA gene of a DGCC7710 strain into which the manN gene has previously been replaced by a manN gene as defined in SEQ ID NO:206 (i.e., of the DGCC7710-IID^{Man}₂₈ strain) leads to a DGCC7710-IID^{Man}₂₈ derivative, which, when used to ferment milk as assayed by test B, exhibits a ratio of the amount of galactose released (mM) over the amount of remaining lactose (mM) in said fermented milk which is more than 1.2, more than 1.5, more than 2, more than 2.5 or more than 3.

For any of b) to d), the DGCC7710-IIAB^{Man}₃₀₅ derivative, the DGCC7710-IIC^{Man}₂₀₈ derivative or the DGCC7710-IID^{Man}₂₈ derivative can further be characterized by the fact it exhibits a ratio of beta-galactosidase activity as assayed by test D over the glucokinase activity as assayed by test E of at least 4·10⁻⁶ as defined herein.

In this context, the term “derivative” when applied to the DGCC7710-IIAB^{Man}₃₀₅ strain, the DGCC7710-IIC^{Man}₂₀₈ strain and the DGCC7710-IID^{Man}₂₈ strain means the DGCC7710-IIAB^{Man}₃₀₅ strain, the DGCC7710-IIC^{Man}₂₀₈ strain and the DGCC7710-IID^{Man}₂₈ strain into which the original ccpA gene (the one of the DGCC7710 strain) has been replaced by the mutated ccpA gene to be assayed.

In this context, DGCC7710-IIAB^{Man}₃₀₅ strain means a DGCC7710 strain into which its manL gene is replaced by a manL gene as defined in SEQ ID NO:111. Similarly, DGCC7710-IIC^{Man}₂₀₈ strain means a DGCC7710 strain into which its manM gene is replaced by a manM gene as defined in SEQ ID NO:157. Similarly, DGCC7710-IID^{Man}₂₈ strain means a DGCC7710 strain into which its manN gene is replaced by a manN gene as defined in SEQ ID NO:206.

In an embodiment, the mutated ccpA polynucleotide is not a knocked-out allele (i.e., a disrupted allele) of the ccpA gene.

In an embodiment, the ccpA gene mutation is a mutation in the coding sequence of the ccpA gene, in particular in the first 270 nucleotides of the coding sequence of the ccpA gene. In an embodiment, the mutation is a mutation selected from the group consisting of:
a) a non-sense mutation (i.e. leading to a STOP codon) located between the nucleotide 1 and the nucleotide 270 of the coding sequence of the ccpA gene; and
b) a mutation, located in the first quarter of the coding sequence of the ccpA gene (i.e., between nucleotide 1 and the nucleotide 250), leading to a frameshift of the open reading frame of the ccpA gene.

In an embodiment, the mutation leading to a frameshift of the open reading frame of the ccpA gene is located between nucleotide 50 and the nucleotide 200 of the coding sequence of the ccpA gene. In an embodiment, the mutation leading to a frameshift of the open reading frame of the ccpA gene is located between nucleotide 100 and the nucleotide 150 of the coding sequence of the ccpA gene. Whatever the location of the mutation leading to a frameshift, the mutation is selected from the group consisting of a deletion, an insertion or a deletion/insertion (which all are not a multiple of 3).

In an embodiment, the sequence of the mutated ccpA polynucleotide is selected from the group consisting of a) a sequence as defined in SEQ ID NO:71; and b) a ccpA variant sequence having at least 90% identity with SEQ ID NO:71. The definition of the ccpA variant having at least 90% identity is as detailed under the paragraph “III. Mutations of the ccpA gene” above.

The (mutated) glcK polynucleotide as defined herein [encoding a mutated Streptococcus thermophilus glucokinase as defined herein] and the (mutated) ccpA polynucleotide as defined herein can be used to design a Streptococcus thermophilus strain, in particular a lactose-positive, galactose-negative, Streptococcus thermophilus strain. The use consists in substituting the glcK gene and/or the ccpA gene of an original Streptococcus thermophilus strain by a mutated glcK polynucleotide and/or a mutated ccpA polynucleotide as defined herein, to design a Streptococcus thermophilus strain having its original gene(s) replaced by the (mutated) one(s) as defined herein. The invention is also directed to a method to design a Streptococcus thermophilus strain, comprising 1) substituting the glcK gene and/or the ccpA gene of an original Streptococcus thermophilus strain by a mutated glcK polynucleotide and/or a mutated ccpA polynucleotide as defined herein, and 2) obtaining a Streptococcus thermophilus strain having its original gene(s) replaced by the (mutated) one(s) as defined herein.

In an embodiment of the use or the method, the original Streptococcus thermophilus strain carries a mutation in at least one, in particular one, gene encoding a protein of the mannose-glucose-specific PTS, in particular in its manL gene, in its manM gene and/or in its manN gene as defined herein; thus, the substitution of the glcK gene and/or the ccpA gene of this original Streptococcus thermophilus strain by a mutated glcK polynucleotide and/or a mutated ccpA polynucleotide as defined herein will lead to a lactose-positive, galactose-negative, Streptococcus thermophilus strain of the invention. In another embodiment of the use or the method, the original Streptococcus thermophilus strain does not carry a mutation in a gene encoding a protein of the mannose-glucose-specific PTS; thus, the substitution of the glcK gene and/or the ccpA gene of this original Streptococcus thermophilus strain by a mutated glcK polynucleotide and/or a mutated ccpA polynucleotide as defined herein will lead to an intermediate lactose-positive, galactose-negative, Streptococcus thermophilus strain, which can be used as a starting strain to mutate at least one, in particular one, gene encoding a protein of the mannose-glucose-specific PTS, in particular the manL gene, manM gene and/or manN gene, to design a lactose-positive, galactose-negative, Streptococcus thermophilus strain of the invention.

The invention is also directed to the use of a mutated gene (or polynucleotide) encoding a protein of the mannose-glucose-specific PTS, in particular a mutated manL gene, a mutated manM gene or a mutated manN gene to design a Streptococcus thermophilus strain, in particular a lactose-positive, galactose-negative, Streptococcus thermophilus strain. In an embodiment, the use consists in substituting [or replacing] the manL gene, manM gene or manN gene of an original Streptococcus thermophilus strain respectively by a mutated manL, manM or manN polynucleotide as defined herein, to design a Streptococcus thermophilus strain having its original gene replaced by the (mutated) one as defined herein. By “respectively”, it is meant that the original manL is replaced by the mutated manL, the original manM is replaced by the mutated manM and/or the original manN is replaced by the mutated manN. The invention is also directed to a method to design a Streptococcus thermophilus strain, comprising 1) substituting [or replacing] the manL gene, manM gene or manN gene of an original Streptococcus thermophilus strain respectively by a mutated manL, manM or manN polynucleotide as defined herein, and 2) obtaining a Streptococcus thermophilus strain having its original gene replaced by the (mutated) one as defined herein.

In an embodiment of the use or the method, the original Streptococcus thermophilus strain carries a mutation in its glcK gene. In an embodiment of the use or the method, the original Streptococcus thermophilus strain carries a mutation in its ccpA gene. In an embodiment of the use or the method, the original Streptococcus thermophilus strain carries a mutation in its glcK gene and a mutation in its ccpA gene. It is the aim of the invention that the substitution of the manL gene, manM gene or manN gene in an original Streptococcus thermophilus strain [carrying a mutation in its glcK gene and/or a mutation in its ccpA gene] respectively by a mutated manL, manM or manN polynucleotide as defined herein, will lead to a lactose-positive, galactose-negative, Streptococcus thermophilus strain as defined in the invention, i.e., a lactose-positive, galactose-negative, Streptococcus thermophilus strain exhibiting a ratio of the amount of galactose released (mM) over the amount of remaining lactose (mM) which is more than 1.2, more than 1.5, more than 2 or more than 3 as defined herein.

In an embodiment of the use or the method, the original Streptococcus thermophilus strain carries a mutation in its glcK gene as defined under part I above. In an embodiment of the use or the method, the original Streptococcus thermophilus strain carries a mutation in its ccpA gene as defined under part Ill above. In an embodiment of the use or the method, the original Streptococcus thermophilus strain carries a mutation in its glcK gene as defined under part I above and a mutation in its ccpA gene as defined under part Ill above.

In an embodiment, the mutated Streptococcus thermophilus manL gene, manM gene or manN gene encodes respectively a IIAB^{Man} protein, a IIC^{Man} protein or IID^{Man} protein, the glucose import activity of which is decreased or abolished.

In an embodiment, the mutated Streptococcus thermophilus manL gene, manM gene or manN gene is characterized by the fact that, when individually inserted in lieu of the manL gene, manM gene or manN gene of the DSM32587 strain:
the DSM32587 derivative, which, when used to ferment milk as assayed by test B, exhibits a ratio of the amount of galactose released (mM) over the amount of remaining lactose (mM) in said fermented milk which is more than 1.2. In a particular embodiment, a mutated manL, manM or manN gene is considered to be according to the invention when the ratio of the amount of galactose released (mM) over the amount of remaining lactose (mM) in the fermented milk is more than 1.5, is more than 2 or is more than 3.

In this context, the term “derivative” when applied to the DSM32587 strain, means the DSM32587 strain into which the original manL, manM or manN gene has been replaced by the mutated manL, manM or manN gene to be assayed.

In an embodiment, the mutated Streptococcus thermophilus manL gene, manM gene or manN gene is characterized by the fact that, when individually inserted in lieu of the manL gene, manM gene or manN gene of the a DGCC7710 strain into which the ccpA gene has previously been replaced by the ccpA gene as defined in SEQ ID NO:71 (i.e., of the DGCC7710-ccpA_{Δ1A114-120} strain):
the DGCC7710-ccpA_{Δ1A114-120} derivative, which, when used to ferment milk as assayed by test B, exhibits a ratio of the amount of galactose released (mM) over the amount of remaining lactose (mM) in said fermented milk which is more than 1.2. In a particular embodiment, a mutated manL, manM or manN gene is considered to be according to the invention when the ratio of the amount of galactose released (mM) over the amount of remaining lactose (mM) in the fermented milk is more than 1.5, is more than 2 or is more than 3.

In this context, the expression “DGCC7710-CCpA_{Δ1A114-120} derivative” when applied to the DGCC7710-ccpA_{Δ1A114-120} strain, means the DGCC7710-ccpA_{Δ1A114-120} strain into which the original manL, manM or manN gene has been replaced by the mutated manL, manM or manN gene to be assayed. In this context, DGCC7710-CCpA_{Δ1A114-120} strain is the DGCC7710 strain into which its ccpA gene has previously been replaced by the ccpA gene as defined in SEQ ID NO:71.

By “individually inserted”, it is meant that, for characterization of the mutated man gene, only one man gene of the DSM32587 strain or of the DGCC7710-ccpA_{Δ1A114-120} strain is replaced (or substituted) by the respective mutated man gene to be characterized.

In an embodiment, the mutated gene encoding a protein of the mannose-glucose-specific PTS is a mutated manL gene. In an embodiment, the mutated Streptococcus thermophilus manL codes for a Streptococcus thermophilus IIAB^{Man} protein, the glucose import activity of which is decreased or abolished, in particular a Streptococcus thermophilus IIAB^{Man} protein truncated in position 305 (IIAB^{Man}₃₀₅). In an embodiment, the mutated Streptococcus thermophilus manL codes for a truncated Streptococcus thermophilus IIAB^{Man} protein, the sequence of which is selected from the group consisting of a) a sequence as defined in SEQ ID NO:112 and b) a IIAB variant sequence having at least 90% similarity or identity with SEQ ID NO:112, in particular being 305 amino acids in length. In an embodiment, the mutated manL gene encodes a IIAB^{Man} protein, the sequence of which is selected from the group consisting of SEQ ID NO: 112 to 128. In an embodiment, the mutated Streptococcus thermophilus manL gene is as defined in SEQ ID NO:111. The expression “IIAB^{Man} variant having at least 90% similarity or identity” is a defined in “II. Mutations of a gene encoding a protein of the mannose-glucose-specific PTS, in particular mutations of the manL, manM and manN genes” above.

In an embodiment, the mutated gene encoding a protein of the mannose-glucose-specific PTS is a mutated manM gene. In an embodiment, the mutated Streptococcus thermophilus manM codes for a Streptococcus thermophilus IIC^{Man} protein, the glucose import activity of which is decreased or abolished, in particular a Streptococcus thermophilus IIC^{Man} protein truncated in position 208 (IIC^{Man}₂₀₈). In an embodiment, the mutated Streptococcus thermophilus manM codes for a truncated Streptococcus thermophilus IIC^{Man} protein, the sequence of which is selected from the group consisting of a) a sequence as defined in SEQ ID NO:158 and b) a IIC^{Man} variant sequence having at least 90% similarity or identity with SEQ ID NO:158, in particular being 208 amino acids in length. In an embodiment, the mutated manM gene encodes a IIC^{Man} protein, the sequence of which is selected from the group consisting of SEQ ID NO:158 to 165. In an embodiment, the mutated Streptococcus thermophilus manM gene is as defined in SEQ ID NO:157. The expression “IIC^{Man} variant having at least 90% similarity or identity” is a defined in “II. Mutations of a gene encoding a protein of the mannose-glucose-specific PTS, in particular mutations of the manL, manM and manN genes” above.

In an embodiment, the mutated gene encoding a protein of the mannose-glucose-specific PTS is a mutated manN gene. In an embodiment, the mutated Streptococcus thermophilus manN codes for a Streptococcus thermophilus IID^{Man} protein, the glucose import activity of which is decreased or abolished, in particular a Streptococcus thermophilus IID^{Man} protein truncated in position 28 (IID^{Man}₂₈). In an embodiment, the mutated Streptococcus thermophilus manN codes for a truncated Streptococcus thermophilus IID^{Man} protein, the sequence of which is selected from the group consisting of a) a sequence as defined in SEQ ID NO:207; and b) a IID^{Man} variant sequence having at least 90% similarity or identity with SEQ ID NO:207, in particular being 28 amino acids in length. In an embodiment, the mutated manN gene encodes a IID^{Man} protein, the sequence of which is selected from the group consisting of SEQ ID NO: 207 to 211. In an embodiment, the mutated Streptococcus thermophilus manN gene is as defined in SEQ ID NO:206. The expression “IID^{Man} variant having at least 90% similarity or identity” is a defined in “II. Mutations of a gene encoding a protein of the mannose-glucose-specific PTS, in particular mutations of the manL, manM and manN genes” above.

In an embodiment, the polynucleotide as defined herein is provided under an isolated form. An “isolated” polynucleotide, is substantially or essentially free from components that normally accompany or interact with the gene as found in its naturally occurring environment. Thus, an isolated polynucleotide is substantially free of other cellular material, or culture medium when produced by recombinant techniques, or substantially free of chemical precursors or other chemicals when chemically synthesized.

The invention is also directed to a construct comprising a polynucleotide as defined herein. In an embodiment, the present invention covers a construct comprising a polynucleotide of the invention operably linked to a regulatory sequence. The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences. The term “regulatory sequences” includes promoters and/or enhancers and other expression regulation signals. The term “promoter” is used in the normal sense of the art, e.g. an RNA polymerase binding site. In an embodiment, independently or in combination with the “regulatory sequence” embodiment, the construct contains or expresses another gene, such as a marker allowing for the selection of the construct. Various markers exist which may be used, for example those markers that provide for antibiotic resistance—e.g. resistance to bacterial antibiotics—such as Erythromycin, Ampicillin, Streptomycin and Tetracycline.

Thus, in a further aspect, there is provided a vector comprising a polynucleotide or a construct as defined herein. As used herein, the term “vector” refers to any nucleic acid molecule into which another nucleic acid (e.g., the polynucleotide of the invention) can be inserted and which can be introduced into and replicate within bacterial strain such as Streptococcus thermophilus strain. Thus, the term refers to any nucleic acid construct (and, if necessary, any associated delivery system) capable of use for introducing genetic material into a bacterial strain, in particular a Streptococcus thermophilus strain. Selection of appropriate vectors is within the knowledge of those having skill in the art. In an embodiment, the vector is a plasmid. As used herein, the term “plasmid” refers to a circular double-stranded (ds) DNA construct that can be used as a vector for introducing DNA into a bacterial strain, in particular a Streptococcus thermophilus strain. The constructs or the vectors may be introduced into a bacterial strain as described herein, such as the DGCC7710 strain.

The polynucleotide, construct, vector or plasmid of the invention disclosed herein can be introduced into a Streptococcus thermophilus strain, using any method available.

“Introducing” (and “introduced”) is intended to mean presenting to the Streptococcus thermophilus strain, the polynucleotide, construct, vector or plasmid of the invention as defined herein, in such a manner that the component(s) gains access to the interior of the Streptococcus thermophilus strain. The methods and compositions do not depend on a particular method for introducing a sequence into a Streptococcus thermophilus strain, only that the polynucleotide, construct, vector or plasmid of the invention gains access to the interior of the Streptococcus thermophilus strain. Introducing includes the incorporation of a polynucleotide, construct, vector or plasmid of the invention into the Streptococcus thermophilus strain where polynucleotide or construct of the invention may be incorporated into the genome of the Streptococcus thermophilus strain, and includes the transient (direct) provision of a polynucleotide or construct to the Streptococcus thermophilus strain.

Introducing a polynucleotide, construct, vector or plasmid of the invention into a Streptococcus thermophilus strain can be carried out by several methods, including transformation, conjugation, transduction or protoplast fusion. Methods for introducing polynucleotide, construct, vector or plasmid of the invention by transformation into a Streptococcus thermophilus strain, include, but are not limited to, microinjection, electroporation, stable transformation methods, transient transformation methods [such as induced competence using chemical (e.g. divalent cations such as CaCl₂) or mechanical (electroporation) means], ballistic particle acceleration (particle bombardment), direct gene transfer, viral-mediated introduction, cell-penetrating peptides or mesoporous silica nanoparticle (MSN)-mediated direct protein delivery. Introducing a polynucleotide, construct, vector or plasmid of the invention into a Streptococcus thermophilus strain can be carried out by conjugation, which is a specific method of natural DNA exchange requiring physical cell-to-cell contact. Introducing a polynucleotide, construct, vector or plasmid of the invention into a Streptococcus thermophilus strain can be carried out by transduction, which is the introduction of DNA via a virus (e.g. phage) infection which is also a natural method of DNA exchange. Generally, such methods involve incorporating a polynucleotide within a viral DNA or RNA molecule.

The invention is also directed to the following mutated man genes as such and their corresponding encoded proteins:
the mutated Streptococcus thermophilus manL coding for a Streptococcus thermophilus truncated IIAB^{Man} protein, the sequence of which is selected from the group consisting of a) a sequence as defined in SEQ ID NO:112 and b) a IIAB variant sequence having at least 90% similarity or identity with SEQ ID NO: 112, in particular being 305 amino acids in length. In an embodiment, the mutated Streptococcus thermophilus manL gene encodes a IIAB^{Man} protein, the sequence of which is selected from the group consisting of SEQ ID NO: 112 to 128. In an embodiment, the mutated Streptococcus thermophilus manL gene is as defined in SEQ ID NO:111.
the mutated Streptococcus thermophilus manN coding for a Streptococcus thermophilus truncated IID^{Man} protein, the sequence of which is selected from the group consisting of a) a sequence as defined in SEQ ID NO:207; and b) a IID^{Man} variant sequence having at least 90% similarity or identity with SEQ ID NO:207, in particular being 28 amino acids in length. In an embodiment, the mutated Streptococcus thermophilus manN gene encodes a IID^{Man} protein, the sequence of which is selected from the group consisting of SEQ ID NO: 207 to 211. In an embodiment, the mutated Streptococcus thermophilus manN gene is as defined in SEQ ID NO:206.

The expression “IIAB^{Man} variant and IID^{Man} variant having at least 90% similarity or identity” is a defined in “II. Mutations of a gene encoding a protein of the mannose-glucose-specific PTS, in particular mutations of the manL and manN genes” above.

The invention is also directed to a method to design a lactose-positive, galactose-negative, Streptococcus thermophilus strain:
a) providing a lactose-positive, galactose-negative, Streptococcus thermophilus strain, carrying a glcK gene encoding a glucokinase, the activity of which is reduced but not null as defined herein, and optionally carrying a mutated ccpA gene;
b) mutating at least one, in particular one, gene encoding a protein of the mannose-glucose-specific PTS, in particular a manL gene, a manM gene and/or a manN gene; and
c) selecting a lactose-positive, galactose-negative, Streptococcus thermophilus strain, which, when used to ferment milk as assayed by test B, exhibiting a ratio of the amount of galactose released (mM) over the amount of remaining lactose (mM) in said fermented milk is more than 1.2.

In an embodiment, the strain selected in step c) exhibits a ratio of the amount of galactose released (mM) over the amount of remaining lactose (mM) in said fermented milk which is more than 1.5, more than 2, more than 2.5 or more than 3, when assayed by test B.

In an embodiment, the at least one gene encoding a protein of the mannose-glucose-specific PTS, in particular a manL, manM or manN gene, is mutated such as encoding a protein, in particular a truncated protein, the glucose import activity of which is decreased or abolished.

The invention is also directed to a method to design a lactose-positive, galactose-negative, Streptococcus thermophilus strain:
a) providing a lactose-positive, galactose-negative, Streptococcus thermophilus strain, carrying at least one gene, in particular one gene, encoding at least one mutated protein, in particular one protein, of the mannose-glucose-specific PTS, in particular a mutated IIAB^{Man} protein, a mutated IIC^{Man} protein and/or a mutated IID^{Man} protein, the glucose import activity of which is decreased or abolished;
b) mutating a glcK gene encoding a glucokinase, the activity of which is reduced but not null as defined herein, and/or mutating a ccpA gene;
c) selecting a lactose-positive, galactose-negative, Streptococcus thermophilus strain, which, when used to ferment milk as assayed by test B, exhibiting a ratio of the amount of galactose released (mM) over the amount of remaining lactose (mM) in said fermented milk is more than 1.2.

In an embodiment, the strain selected in step c) exhibits a ratio of the amount of galactose released (mM) over the amount of remaining lactose (mM) in said fermented milk which is more than 1.5, more than 2, more than 2.5 or more than 3, when assayed by test B.

In an embodiment, the gene encoding a mutated protein of the mannose-glucose-specific PTS the glucose import activity of which is decreased or abolished, encodes a truncated protein, in particular a truncated IIAB^{Man} protein, a truncated IIC^{Man} protein or a truncated IID^{Man} protein.

In an embodiment of the 2 methods defined above, the method further comprises: d) selecting a strain, which when used for fermenting milk by test B, provides a low lactose fermented milk, in particular a fermented milk, the amount of remaining lactose of which is less than 60 mM, less than 50 mM, less than 45 mM, less than 40 mM, less than 35 mM or less than 30 mM

In an embodiment of the 2 methods defined above, the method further comprises
d) selecting a strain which, when inoculated to a milk which is then fermented by test B, the pH of the milk decreases to a pH at which the speed of acidification definitively becomes less than 0.1 mUpH/min, wherein said pH_{STOP} is comprised between 4.6 and 5.3, and optionally the slope between pH6 and pH5.5 is at least −0.008 UpH/min. In an embodiment, the strain is selected when the pH_{STOP} is comprised between a minimal value selected from the group consisting of 4.6, 4.7 and 4.8 and a maximal value selected from the group consisting of 5.1, 5.2 and 5.3. In an embodiment, the strain is selected when the slope between pH6 and pH5.5 is at least 0.009 or at least 0.01 UpH/min.
   Various preferred features and embodiments of the present invention will now be described by way of non-limiting examples.

### Material and Methods

### Strains and Growth Conditions

The S. thermophilus strains (ST) disclosed in the present application were grown at 37° C. in M17 broth (Oxoïd, supplier reference CM0817) supplemented with 30 g/L of appropriate carbohydrate and if necessary, addition of 15 g/L Agar bacteriologic Type A (Biokar, supplier reference #A1010HA), or at 43° C. in milk (UHT semi-skimmed milk “Le Petit Vendéen”+3% milk powder BBA Lactalis). Autoclaved M17 broth was supplemented with 0.2 μm filtered lactose, sucrose, galactose or glucose. Frozen stocks of ST strains were obtained by half-diluting an overnight culture in M17 supplemented with 5 g/L lactose, and 10% glycerol, and stored at −20° C.

### Quantification of Carbohydrate Catabolism During Milk Fermentation [Test B]

UHT semi-skimmed milk “Le Petit Vendeen (“yoghurt milk”) containing 3% (w/v) milk powder (BBA, Lactalis), previously pasteurized 10 min at 90° C., was inoculated at 1% (v/v, about 10⁷ CFU/ml) with a culture of the S. thermophilus strain to be assayed (M17-carbohydrate-free resuspended cells from overnight culture grown in M17 supplemented 3% sucrose). This milk was found to contain around 175 mM of lactose. The inoculated milk flasks were statically incubated in a water bath at 43° C. during 24h, to obtain fermented milk. T0 samples and samples of fermented milk (T24h) (5 g) were diluted in 25 g 0.025 N H₂SO₄, before being centrifuged at 4600 rpm for 10 minutes at 4° C. The supernatant was filtered through a 0.2 μm Nylon filter (Phenomenex, Germany, Aschaffenburg) directly into a 2 ml HPLC vial. Samples were stored at −20° C. until further analysis. Carbohydrates were quantified by high performance liquid chromatography (Agilent 1200 HPLC) equipped with a refractive index detector using an AMINEX™ HPX-87H anion exchange column (Bio-Rad Laboratories Inc.) at 35° C., with 12.5 mM H₂SO₄ as the elution fluid and a flow rate of 0.6 ml min⁻¹. The exploitation of results was made with CHEMSTATION® reprocessing software (Agilent).

glcK Sequencing

PCR amplification of the glucokinase gene was performed using primers GlcK-F4 (5′-CAGGTATGAGTTTAGCAACGG-3′) (SEQ ID NO: 212) and GlcK-R12 (5′-ATTCACCACGGCCTGAGAC-3′) (SEQ ID NO: 220), [incubation step at 98° C., 5 min, followed by 33 cycles of 98° C., 45 s; 58° C., 30 s; 68° C., 3 min, with a final extension step at 72° C., 7 min]. The PCR products of 2788 bp were then treated with Illustra™ ExoProStar™ according to the manufacturer's instructions (GE Healthcare). Sequencing reactions were performed by using the BigDye® Terminator v3.1 Cycle Sequencing kit (Life Technologies) according to the manufacturer's instructions using an AB3500 (Applied Biosystems™), and primers listed in Table 2.

TABLE 2
Listing of primers for sequencing glcK.

| Primers | Sequences |
|---|---|
| GlcK-F4 (SEQ ID NO: 212) | CAGGTATGAGTTTAGCAACGG |
| GlcK-R8 (SEQ ID NO: 213) | AGTTCAATCTTCATCATCTCG |
| GlcK-F5 (SEQ ID NO: 214) | GTAGCCACATTGTTCCTGAC |
| GlcK-R6 (SEQ ID NO: 215) | TTGCTGAAGCTACAGTTTCC |
| GlcK-R4 (SEQ ID NO: 216) | TAAGCAAGACTAGCAGCTCC |
| GlcK-F7 (SEQ ID NO: 217) | TTGCGTAGTCGTGTTGAAGG |
| GlcK-R10 (SEQ ID NO: 218) | ATTGTCCCTTCATAAGCATCG |
| GlcK-F11 (SEQ ID NO: 219) | CGAACTGGGTGCAGATGATG |
| GlcK-R12 (SEQ ID NO: 220) | ATTCACCACGGCCTGAGAC |

### Glucokinase Activity [Test A]

A fresh overnight culture of a Streptococcus thermophilus strain in M17 containing 30 g/L lactose was obtained and used to inoculate at 1% (vol/vol) 10 ml of fresh M17 30 g/L lactose. Cells were harvested by centrifugation (6000 g, 10 min, 4° C.) at a 600 nm optical density (OD600) of 0.8, washed in 5 ml cold GLCK buffer (5 mM MgCl2, 10 mM K₂HPO₄/KH₂PO₄ [pH 7.2]), and resuspended in 500 μl cold GLCK buffer. EDTA-free protease inhibitors “cOmplete™” (Roche, supplier reference 04693132001) was added in GLCK buffer as described by the provider. Cells were disrupted by the addition of 100 mg glass beads (150-212 μm, Sigma G1145) to 200 μl resuspended cells and oscillation at a frequency of 30 cycles/s for 6 min in a MM200 oscillating mill (Retsch, Haan, Germany). Cell debris and glass beads were removed by centrifugation (14000 g, 15 min, 4° C.), and supernatant transferred into a clean 1.5 mL centrifuge tube kept on ice. Total protein content was determined by using the FLUKA Protein Quantification Kit-Rapid (ref 51254). The glucokinase activity in the cell extracts was determined spectrophotometrically by a glucose-6-phosphate dehydrogenase (G-6PDH, EC1.1.1.49):NADPH-coupled assay (Porter et al., 1982), essentially as described by Pool et al. (2006). Each sample (5, 10 and 20 μL) was added to assay buffer (10 mM K₂HPO₄/KH₂PO₄ [pH 7.2], 5 mM MgCl2, 1 mM ATP, 20 mM glucose, 1 mM NADP, 1 U G-6PDH) in a 250 μL final volume, and the mixture was left for 5 min at 30° C. The optical density at 340 nm was measured for 5 minutes by using a Synergy HT multi-detection microplate reader (BIO-TEK). One unit of glucokinase corresponds to the amount of enzyme that catalyzes the phosphorylation of 1 μmole of D-glucose to D-Glucose 6-phosphate per minute under the assay conditions. Glucokinase activity was calculated as follows:

Glucokinase activity (U/g of total protein extract)=dOD×V/[dt×l×ε×Qprot], wherein:
dOD is the variation of optical density (OD) at 340 nm
V is the volume of the reaction (herein 250 μL)
dt=measurement time (in minutes)
l=optical path length (herein 0.73 cm)
ε=molar attenuation coefficient of NADPH; H+(herein 6220 cm2/μmol)
Qprot=quantity of protein in the cuvette (in g)
   Measurements were triplicated for each sample, and the glucokinase specific activity values given herein under test A are the mean of three independent experiments.

### Maximal Forward Velocity of GlcK [Test C]

The maximal forward velocity (Vmax) of GlcK was determined by using various concentrations of glucose (0, 5, 10, 15, 20 mM) on crude extract prepared as described in the “glucokinase activity” (test A). Measurements were triplicated for each sample, and the Vmax values given herein under test C are the mean of three independent experiments. The linear regression representing the inverse of the specific velocity in function of the inverse of the glucose concentration gives the inverse of the maximal forward velocity at the intersection with the Y-axis of the graphic.

### Milk Acidifying Performance

The acidifying properties of S. thermophilus strains were evaluated by recording the pH over time, during milk fermentation as described in test B. The pH was monitored for 24 hours using the CINAC system (Alliance Instruments, France; pH electrode Mettler 405 DPAS SC, Toledo, Spain) as previously described. The pH was measured and recorded every 5 minutes. Using the CINAC v2.07 software, the slope between pH 6.0 and pH 5.5 (UpH/minute) [Slope pH6-5.5] was calculated.
Transfer of the glcK Allele of the ST0 Strain into the Genome of 3 Other S. thermophilus Strains
A 1889 bp PCR product bearing the glcK gene of the ST0 strain was obtained using primers GlcK-F1 (5′-GAAGCAGTTTGGGGTAGTAG-3′) (SEQ ID NO: 221) and GlcK-R2 (5′-GAGTTATCTACAGGAGCTGG-3′) (SEQ ID NO: 222). The PCR product was then purified using QIAquick® PCR Purification Kit (Qiagen), and eluted in RNase free water. The concentration of the PCR product was determined using NanoDrop™ 2000 spectrophotometer (Thermo Scientific, Wilmington, MA). The size and the purity of the PCR product were verified by gel-based capillary electrophoresis QIAxcel® system (Qiagen, Hilden, Germany). Strains DGCC7710, ST1.1 and ST1.2 were transformed with the 1889 bp PCR product and mutants having their glcK gene replaced by the glcK allele of the ST0 strain were selected (the presence of the glcK allele of the ST0 strain was checked by sequencing).

### Results

### Example 1: Screening of a Streptococcus thermophilus Collection for Glucose-Excreting Strains

The inventors of the present application, with a wish to select strains secreting glucose, used another approach. A Streptococcus thermophilus collection was screened by test B for strains able to excrete glucose in the fermented milk. An amount of 10 mM of glucose was used as the minimal threshold for selection. One strain, ST0, releasing 30 mM of glucose in fermented milk using test B, was selected.

### Example 2. Identification of a Mutation in the glcK Gene

Sequencing of several genes—of the ST0 strain—known to be involved in the catabolism of carbohydrates in S. thermophilus was carried out and aligned with the corresponding gene sequences of other Streptococcus thermophilus of our collection.

A non-conservative amino acid difference, E275K, was identified in the GlcK sequence of the ST0 strain, which was not found in any of the GlcK sequence of the other S. thermophilus strains of the collection; this amino acid difference is the result of a A at position 823 of the glcK gene instead of a G. Further comparison of the glucokinase encoded by the glcK gene of other S. thermophilus strains confirmed that a lysine at position 275 (instead of a glutamic acid) was unique to ST0 and was not found in any of the 107 other strains.

Other amino acid differences identified in the deduced glucokinase from these 108 strains are represented in Table 3. Thus, 10 different glucokinase types could be distinguished (GlcK type 1 to GlcK type 10, as set forth respectively in SEQ ID NOs: 2, 4, 6, 8, 10, 12, 14, 16, 18 and 20). For the next experiments, 10 strains, ST1 to ST10, each expressing a unique glucokinase were selected. SEQ ID NO:2 was taken as a reference sequence, because this GlcK type was found in about 70% of the 108 analysed strains. In particular, the DGCC7710 strain, deposited at the DSMZ under accession number DSM28255 on Jan. 14, 2014, encodes a glucokinase as defined in SEQ ID NO:2. It is noteworthy that the only amino acid difference between the sequence of the glucokinase encoded by the ST0 strain and SEQ ID NO:2 is the amino acid difference in position 275.

TABLE 3
GlcK protein differences identified by comparison of the GlcK protein sequence
encoded by 108 S. thermophilus, and GlcK protein sequence encoded by ST0. The listed
amino acid positions indicate all the amino acid differences between the GlcK proteins. GlcK
protein sequence from ST1 (SEQ ID NO: 2) was chosen as a reference sequence. The amino
acid positions not listed in this Table are identical for all the glucokinases from this study.
“aa diff” column gives the number of amino acid differences as compared to SEQ ID NO: 2.
“% id” column gives the percentage of identity to SEQ ID NO: 2

| aa position | 21 | 33 | 38 | 49 | 64 | 100 | 131 | 133 | 137 | 141 | 144 | 149 | 152 |
|---|---|---|---|---|---|---|---|---|---|---|---|---|---|
| ST1 | T | E | E | E | D | V | N | N | I | T | G | A | N |
| ST2 | — | — | — | — | — | — | — | — | V | — | | — | — |
| ST3 | N | G | | — | — | — | — | — | V | — | | V | K |
| ST4 | — | — | — | — | A | — | — | — | — | — | | — | — |
| ST5 | — | — | — | — | — | — | — | — | — | — | | — | — |
| ST6 | — | — | — | — | — | — | — | — | — | — | | — | — |
| ST7 | — | — | — | D | — | — | — | — | V | — | | — | — |
| ST8 | N | G | — | — | — | — | — | — | V | — | | V | K |
| ST9 | — | — | D | — | — | — | S | D | V | — | | — | — |
| ST10 | — | — | D | — | — | I | — | D | V | — | | — | — |
| ST0 | — | — | — | — | — | — | — | — | — | — | | — | — |
| ST20 | — | — | — | — | — | — | — | — | — | — | S | — | — |

| aa position | 182 | 188 | 197 | 198 | 209 | 220 | 227 | 252 | 265 | 275 | aa diff. | % id. |
|---|---|---|---|---|---|---|---|---|---|---|---|---|
| ST1 | K | V | V | A | S | F | I | T | V | E | / | / |
| ST2 | — | — | — | — | — | — | — | — | — | — | 1 | 99.7 |
| ST3 | — | — | I | — | — | — | L | — | — | — | 7 | 97.8 |
| ST4 | — | — | — | — | — | — | — | — | — | — | 1 | 99.7 |
| ST5 | — | A | — | — | — | — | — | — | — | — | 1 | 99.7 |
| ST6 | N | — | — | T | — | — | — | — | — | — | 2 | 99.4 |
| ST7 | — | — | — | — | — | L | L | I | — | — | 5 | 98.4 |
| ST8 | — | — | I | — | — | — | — | — | — | — | 6 | 98.1 |
| ST9 | — | — | — | — | — | L | L | I | — | — | 7 | 97.8 |
| ST10 | — | — | — | — | — | L | L | I | I | — | 8 | 97.5 |
| ST0 | — | — | — | — | — | — | — | — | — | K | 1 | 99.7 |
| ST20 | — | — | — | — | — | — | — | — | — | — | 1 | 99.7 |

### Example 3. Measurement of the Glucokinase Activity of the ST0 Strain and Comparison with Other Strains (ST to ST10)

The glucokinase activity of the ST0 strain was compared with the glucokinase activity of the ST1 to ST10 strains selected as reported in example 2, using test A. The results are summarized in Table 4.

TABLE 4
Glucokinase specific activity of 11 strains: 10 strains-ST1
to ST10-each representing a different GlcK protein variant,
and ST0 identified in example 1; ND: not determined

| | Glucokinase specific activity | | |
|---|---|---|---|
| | (U/g total protein extract) | | relative activity |
| Strain | Average | STD | (% DGCC7710) |
| DGCC7710 | 2756 | 140 | 100 |
| (ST1 type) | | | |
| ST2 | 2250 | 381 | 82 |
| ST3 | 2595 | 173 | 94 |
| ST4 | 2014 | 105 | 73 |
| ST5 | ND | ND | ND |
| ST6 | 2892 | 354 | 105 |
| ST7 | 2791 | 104 | 101 |
| ST8 | 2471 | 293 | 90 |
| ST9 | 2553 | 337 | 93 |
| ST10 | 2718 | 280 | 99 |
| ST0 | 977 | 29 | 35 |

These data show that the glucokinase activity of strains ST1 to ST10 is comprised from 2014 to 2791 U/g total protein extract as assayed by test A. A glucokinase activity above 1800 U/g total protein extract was considered to represent a normal glucokinase activity. The glucokinase activity of the DGCC7710 strain was considered as a reference glucokinase activity (since expressing the most frequent GlcK type, defined as SEQ ID NO:2).

In contrast, the ST0 strain, expressing a GlcK protein bearing a lysine at position 275 has a glucokinase activity which is around 977 U/g total protein extract as assayed by test A, i.e. is about 3 times less the glucokinase activity of the DGCC7710 strain (35%).

These data show that the approach retained by the inventors enabled to select for the first time a galactose-negative S. thermophilus strain expressing a glucokinase, the activity of which is significantly reduced but not null.

### Example 4: Comparison of the Glucokinase Activity, Vmax and Km, of the DGCC7710 Strain, a DGCC7710 Derivative Bearing the E275K Difference and a Galactose-Positive Variant of DGCC7710 Bearing a Glucokinase Null Amino Acid Change

A derivative of the DGCC7710 strain was designed, into which the glcK gene encodes a glucokinase with the glutamic acid (E) at position 275 was replaced by the amino acid lysine (K). This derivative (DGCC12534) was deposited at the DSMZ on Aug. 15, 2017 under accession number DSM32587. The sequence of its GlcK protein is as defined in SEQ ID NO:22.

In parallel, a mutant of DGCC7710 was generated, into which the serine (S) at position 72 was replaced by a proline (P), to give a GlcK protein with a sequence as defined in SEQ ID NO:23 [S72P amino acid substitution; reported in strain DSM25851 of application WO2013/160413]. Because the S72P amino acid substitution leads to a null glucokinase activity (i.e. a strain which is not able to use glucose via the glucokinase), the mutant was previously rendered galactose positive (in order to be able to use galactose, because a galactose-negative S. thermophilus strain displaying a null glucokinase activity is expected to be non-viable on lactose). Thus, the gal operon promoter of the DGCC7710 strain was previously mutated according to application WO2011/026863 to give a gal operon promoter having the sequence as defined in SEQ NO:24. A galactose-positive mutant bearing the S72P amino acid substitution in the GlcK protein was obtained and called ST1 m-glcK0-gal+.

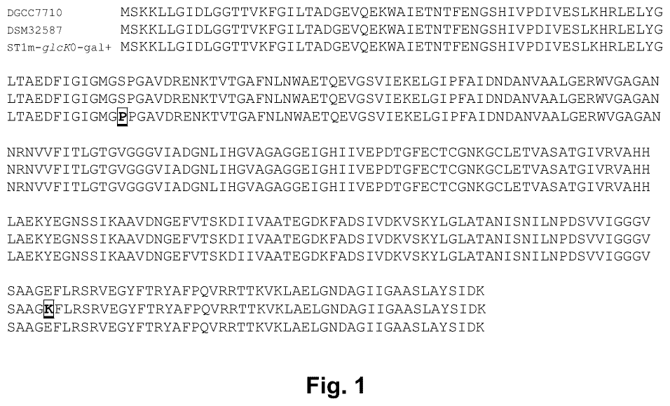

An alignment of the protein sequence of the glucokinase of DGCC7710, DSM32587 and ST1 m-glcK0-gal+ strains is disclosed in FIG. 1.

The glucokinase activity, the Vmax and Km of the glucokinase of DGCC7710, of DSM32587 and of ST1 m-glcK0-gal+ strains were determined as described in the Material and Methods. The results obtained are disclosed in Tables 5 and 6.

TABLE 5
Glucokinase specific activity of DGCC7710 strain, and in
DSM32587 and ST1m-glcK0-gal+ strains and % of activity as
compared to the glucokinase activity of DGCC7710 strain

| | Glucokinase | | |
|---|---|---|---|
| | specific activity | | |
| | (U/g total | | |
| | protein extract) | | relative activity |
| Strain | Average | STD | (% DGCC7710) |
| DGCC7710 | 2756 | 140 | 100 |
| DSM32587 | 907 | 32 | 33 |
| ST1m-glK0-gal+ | 0 | / | 0 |

TABLE 6
Vmax of glucokinase of DGCC7710 strain and in DSM32587
and ST1m0gal+ strains and % of Vmax glucokinase
as compared to the Vmax of glucokinase of DGCC7710 strain,
and Km activity of glucokinase of DGCC7710,
DSM32587 and ST1m-g/cK0-gal+ strains; ND: not determined.

| | Vmax Glucokinase | | | Km Glucokinase | |
|---|---|---|---|---|---|
| | (U/g total protein extract) | | | (mM) | |
| Strain | average | STD | % DGCC7710 | average | STD |
| DGCC7710 | 2855 | 178 | 100 | 1319 | 73 |
| DSM32587 | 914 | 21 | 32 | 1328 | 56 |
| ST1m-gkK0-gal+ | ND | / | ND | ND | / |

The data of Table 5 nicely shown that replacing the glutamic acid (E) at position 275 of the GlcK protein by a lysine (K) is sufficient alone to significantly decrease the glucokinase activity from 2756 to 907 U/g (i.e., 33% of DGCC7710 activity) in the DGCC7710 derivative (DSM32587). The data obtained for the ST1 m-glcK0-gal+ mutant confirm that the S72P amino acid change is sufficient to totally abolish the glucokinase activity. Together with the glucokinase activity, the inventors also studied whether the observed decrease of glucokinase activity in the DSM32587 strain was a consequence of a decrease of the affinity (Km) of the glucokinase for its substrate (glucose) and/or a decrease in the maximum forward velocity (Vmax) of the glucokinase. The data of Table 6 confirm that replacing the glutamic acid (E) at position 275 of the GlcK protein by a lysine (K) is sufficient alone to significantly decrease the Vmax of the glucokinase from 2855 to 914 U/g (i.e., 32% of DGCC7710 Vmax) in the DGCC7710 derivative (DSM32587). In absence of a functional glucokinase in the ST1m-glcK0-gal+ mutant, the Vmax could not be determined.

### Example 5: Identification of a Further GlcK Mutated Protein

A Streptococcus thermophilus strain (ST20) was identified, the glcK gene of which contains a non-conservative amino acid difference, G144S. This amino acid change was not found in any of the GlcK sequence of the other S. thermophilus strains of the collection (GlcK types ST1 to ST10). It is noteworthy that the only amino acid difference between the sequence of the glucokinase encoded by the ST20 strain and SEQ ID NO:2 is the amino acid difference in position 144 (Table 3).

### Example 6: Comparison of Sugar Catabolism and Acidification Kinetics of the DGCC7710 Strain, the DSM32587 Strain, the ST20 Strain, the ST1 m-glcK0-Gal+ Strain, the ST1.1 Strain and a glcK-Mutated ST1.1 Strain, During Milk Fermentation

Considering, on the one hand, the impact of the mutations E275K and G144S on the activity of glucokinase (reduced but not null), and on the other hand, the role of the glucokinase in glucose metabolism, the DGCC7710 strain, the DSM32587 strain, the ST20 strain, the ST1 m-glcK0-gal+ strain (all described above), as well as a second parental strain (ST1.1 strain, encoding a glucokinase as defined in SEQ ID NO:22) and its glcK-mutated ST1.1 strain (having the E275K substitution; ST1.1 m-glcK strain) were used to ferment milk by test B and the concentration of glucose, galactose and lactose present in the fermented milk was determined (see material and methods). The results are summarized in Table 7 below.

TABLE 7
Carbohydrate catabolism in milk fermented (24 h) with DGCC7710,
DSM32587, ST20 and ST1m-g/cK0-gal+. strains

| | Glucose | Galactose | Lactose | slope pH |
|---|---|---|---|---|
| | released | released | remaining | 6-5.5 |
| Strain | (mM) | (mM) | (mM) | (UpH/min) |
| DGCC7710 | <0.1 | 53 | 116 | −0.0140 |
| DSM32587 | 32 | 73 | 95 | −0.0148 |
| ST20 | 49 | 62 | 101 | −0.0124 |
| ST1m-gkK0-gal+ | 109 | 88 | 17 | −0.0077 |
| ST.1.1 | <0.1 | 44 | 119 | −0.0180 |
| ST1.1m-gkK | 35 | 65 | 96 | −0.0168 |

The data of Table 7 show that:
the DSM32587 strain and ST20 strain release 32 mM and 49 mM of glucose respectively after milk fermentation, whereas the DGCC7710 strain having a high glucokinase activity does not release detectable amount of glucose. This difference is also seen between the ST1.1 strain and its glcK mutant (ST1.1 m-glcK). As far as ST1 m-glcK0-gal+ mutant is concerned, this galactose-positive mutant excretes 109 mM of glucose;
the DSM32587 strain and ST20 strain releases 73 mM and 62 mM of galactose respectively after milk fermentation, whereas the DGCC7710 strain releases 53 mM. This slight difference is also seen between the ST1.1 strain and its glcK mutant (ST1.1m-glcK);
with regards to the lactose remaining in the fermented milk, the ST1m-glcK0-gal+ mutant consumes almost all the lactose present in milk (158 mM out of about 175 mM), such that the concentration of remaining lactose in the fermented milk is 17 mM. In contrast, with the DSM32587 strain or the ST20 strain, the concentration of remaining lactose is in the same range as for the DGCC7710 strain [95 and 101 versus 116]. This is also seen between the ST1.1 m-glcK strain and the ST1.1 strain [96 versus 119].

These data show that almost all the galactose moiety (coming from the lactose hydrolysis) is released in the fermented milk when using the DGCC7710, the DSM32587, ST20, ST1.1 and ST1.1m-glcK strains (59 mM, 80 mM, 66 mM, 56 mM and 79 mM of lactose consumed for 53 mM, 73 mM, 62 mM, 44 mM and 65 mM of galactose released). It is reminded that 1 mole of lactose gives after hydrolysis 1 mole of glucose and 1 mole of galactose. The glucose moiety was partially consumed by the strain Thus, in a milk containing 175 mM of lactose (test B), this means that there is more lactose remaining in the milk than lactose consumed by the strain.

This behaviour can also be translated by determining the ratio galactose released (mM) in the fermented milk over lactose remaining (mM) in the fermented milk, when assayed by test B. This ratio is 0.452 in DGCC7710, 0.775 in DSM32587, 0.544 in ST20 strain, 0.373 in ST1.1 and 0.672 in ST1.1m-glcK, i.e., below 1 in the above glcK-mutated strains and their parental strains. This ratio represents the efficiency of lactose uptake and hydrolysis in a galactose-negative strain.

### Example 7: Determination of Sugar Catabolism and Acidification Kinetics in Lactose-Positive Galactose-Negative ccpA-Mutated and Man-Mutated S. thermophilus Strains

Based on the observations done in example 6, the inventors have determined the sugar catabolism and acidification kinetics of S. thermophilus strains mutated in the ccpA gene or mutated in a gene encoding a protein of the mannose-glucose-specific PTS (manL, manM or manN gene). The following mutants have been designed in the background of DGCC7710 (ST1) and ST1.1, and used for fermentation by test B:
a DGCC7710 derivative, into which the ccpA gene was replaced by the ccpA gene having a deletion of a nucleotide A in the stretch of 7 nucleotides A at positions 114-120 (SEQ ID NO:71), leading to a frameshift of the open reading frame of the ccpA gene (ST1 m-ccpA);
a DGCC7710 derivative, having a knocked-out ccpA gene (ST1m-KOccpA);
a DGCC7710 derivative, into which the manL gene was replaced by a manL gene with the substitution of the nucleotide G in the nucleotide T at position 916 (SEQ ID NO:111), leading to a stop codon at position 306 of the IIAB^{Man} protein (ST1 m-manL);
a DGCC7710 derivative, into which the manM gene was replaced by a manM gene with a substitution of the nucleotide G in the nucleotide T at position 625 (SEQ ID NO:157), leading to a stop codon at position 209 of the IIC^{Man} protein (ST1 m-manM);
a DGCC7710 derivative, into which the manN gene was replaced by a manN gene with an insertion of a nucleotide A in the stretch of 5 nucleotides A at positions 37-41 (SEQ ID NO:206), leading to a frameshift of the open reading frame and a truncation of the IID^{Man} protein at position 28 (ST1 m-manN);
a ST1.1 derivative, into which the ccpA gene was replaced by a ccpA gene having a deletion of a nucleotide A in the stretch of 7 nucleotides A at positions 114-120 (SEQ ID NO:71) (ST1.1m-ccpA); and
a ST1.1 derivative, into which the manL gene was replaced by a manL gene with the substitution of the nucleotide G in the nucleotide T at position 916 (SEQ ID NO:111) (ST1.1 m-manL).
   The results are summarized in Table 8 below.

TABLE 8
Carbohydrate catabolism in milk fermented (24 h) with ccpA-mutated
and man-mutated strains and their parental strains

| | Galactose | Lactose | Ratio galactose | slope pH |
|---|---|---|---|---|
| | released | remaining | released/lactose | 6-5.5 |
| Strain | (mM) | (mM) | remaining | (UpH/min) |
| DGCC7710 | 53 | 116 | 0.452 | −0.0140 |
| ST1m-ccpA | 60 | 109 | 0.544 | −0.0125 |
| ST1m-KOccpA | 66 | 96 | 0.686 | −0.0084 |
| ST1m-manL | 65 | 101 | 0.643 | −0.0104 |
| ST1m-manM | 68 | 100 | 0.678 | −0.0089 |
| ST1m-manN | 63 | 102 | 0.615 | −0.0095 |
| ST1.1 | 44 | 119 | 0.373 | −0.0180 |
| ST1.1m-ccpA | 51 | 110 | 0.465 | −0.0160 |
| ST1.1m-manL | 50 | 113 | 0.445 | −0.0123 |

These data show that none of the ccpA-mutated and man-mutated strains are suitable to remove lactose during milk fermentation (lactose concentration between 96 and 113 mM).

These data confirm that the values of the ratio galactose released over lactose remaining in the fermented milk obtained with strains mutated in the ccpA gene or in a gene encoding a protein of the mannose-glucose-specific PTS and with their parental strains, are in agreement with the values reported in example 6 above (i.e., a ratio which is less than 1).

### Example 8: Determination of Sugar Catabolism and Acidification Kinetics in Lactose-Positive Galactose-Negative S. thermophilus Strains Both Mutated in a Gene Encoding a Protein of the Mannose-Glucose-Specific PTS and Mutated in a glcK Gene and/or ccpA Gene

The inventors have then designed strains mutated in a gene encoding a protein of the mannose-glucose-specific PTS (manL, manM or manN) and mutated in a glcK gene and/or ccpA gene. Thus, the double-mutated and triple-mutated strains, based on the following mutated genes, were designed, in the background of DGCC7710 (ST1) or in the background of ST1.1.
ST1 m-glcK+manM: DSM32587 derivative (carrying the glcK gene encoding a glucokinase with the substitution E275K; example 2), into which the manM gene was replaced by the mutated manM gene as defined in SEQ ID NO:157;
ST1 m-ccpA+manL: ST1 m-ccpA derivative (DGCC7710 carrying the mutated ccpA gene as defined in SEQ ID NO:71), into which the manL gene was replaced by the mutated manM gene as defined in SEQ ID NO: 111;
ST1 m-ccpA+manM: ST1 m-ccpA derivative (DGCC7710 carrying the mutated ccpA gene as defined in SEQ ID NO:71), into which the manM gene was replaced by the mutated manM gene as defined in SEQ ID NO:157;
ST1 m-ccpA+manN: ST1 m-ccpA derivative, into which the manN gene was replaced by the mutated manN gene as defined in SEQ ID NO:206;
ST1 m-glcK+ccpA+manM: DSM32587 derivative, into which the ccpA gene was replaced by the mutated ccpA gene as defined in SEQ ID NO:71 and the manM gene was replaced by the mutated manM gene as defined in SEQ ID NO:157;
ST1.1 m-glcK+manM: ST1.1 m-glcK derivative (ST1.1 strain carrying the glcK gene encoding a glucokinase with the substitution E275K), into which the manM gene was replaced by the mutated manM gene as defined in SEQ ID NO:157; and
ST1.1 m-ccpA+manL: ST1.1 m-ccpA derivative (ST1.1 carrying the mutated ccpA gene as defined in SEQ ID NO:71), into which the manL gene was replaced by the mutated manL gene as defined in SEQ ID NO:111.
   The results are summarized in Table 9 below.

TABLE 9
Carbohydrate catabolism in milk fermented (24 h) with double
and triple mutated strains and their parental strains

| | | | Ratio | slope |
|---|---|---|---|---|
| | | | galactose | pH |
| | Galactose | Lactose | released/ | 6-5.5 |
| | released | remaining | lactose | (UpH/ |
| Strain | (mM) | (mM) | remaining | min) |
| DGCC7710 | 53 | 116 | 0.452 | −0.0140 |
| DSM32587 | 73 | 95 | 0.775 | −0.0148 |
| ST1m-glcK+ manM | 117 | 33 | 3.500 | −0.0104 |
| ST1m-ccpA+ manL | 131 | 25 | 5.252 | −0.0089 |
| ST1m-ccpA+ manM | 129 | 31 | 4.207 | −0.0082 |
| ST1m-ccpA+ manN | 128 | 28 | 4.606 | −0.0109 |
| ST1m- | 129 | 29 | 4.431 | −0.0092 |
| glcK+ ccpA+ manM | | | | |
| ST1.1 | 44 | 119 | 0.373 | −0.0180 |
| ST1.1m- | 98 | 55 | 1.780 | −0.0140 |
| glcK+ manM | | | | |
| ST1.1m-ccpA+ manL | 117 | 34 | 3.460 | −0.0091 |

Surprisingly, whereas all the galactose-negative single-mutated strains (mutated in one gene among the glcK gene, the ccpA, the manL gene, the manM gene and the manN gene) show lactose concentration around or more than 100 mM by test B (between 95 and 113) [see tables 7 and 8], the introduction of 2 or 3 mutated genes (a mutated gene encoding a protein of the mannose-glucose-specific PTS, and a mutated glcK gene and/or a mutated ccpA gene) leads to strains which when used to ferment milk by test B remove between 68% and 85% of the lactose originally contained in the milk (i.e., a lactose concentration between 25 and 55 mM).

Interestingly, the ratio galactose released over lactose remaining in the fermented milk defined above is dramatically increased as compared to, not only the non-mutated strains but also the single-mutated strains. Thus, this ratio is comprised between 1.780 and 5.252. This demonstrates that more than the lactose concentration remaining in the fermented milk, the ratio galactose released over lactose remaining in the fermented milk is an excellent parameter to distinguish galactose-negative strains which are suitable for removing lactose during milk fermentation from strains which are not suitable for removing lactose. This shows the interest of the double or triple mutant strains for manufacturing producers desiring to obtain low lactose fermented milk products (with less than 60 mM).

### Example 9: pH Stop in Milk Fermented with the Double or Triple Mutants of the Invention

The DGCC7710, ST1 m-glcK+manM, ST1 m-ccpA+manL, ST1 m-ccpA+manM, ST1 m-ccpA+manN, ST1 m-glcK+ccpA+manM, ST1.1, ST1.1 m-glcK+manM and ST1.1 m-ccpA+manL strains (all described above) were used to ferment milk by test B [24 hours at fermentation temperature].

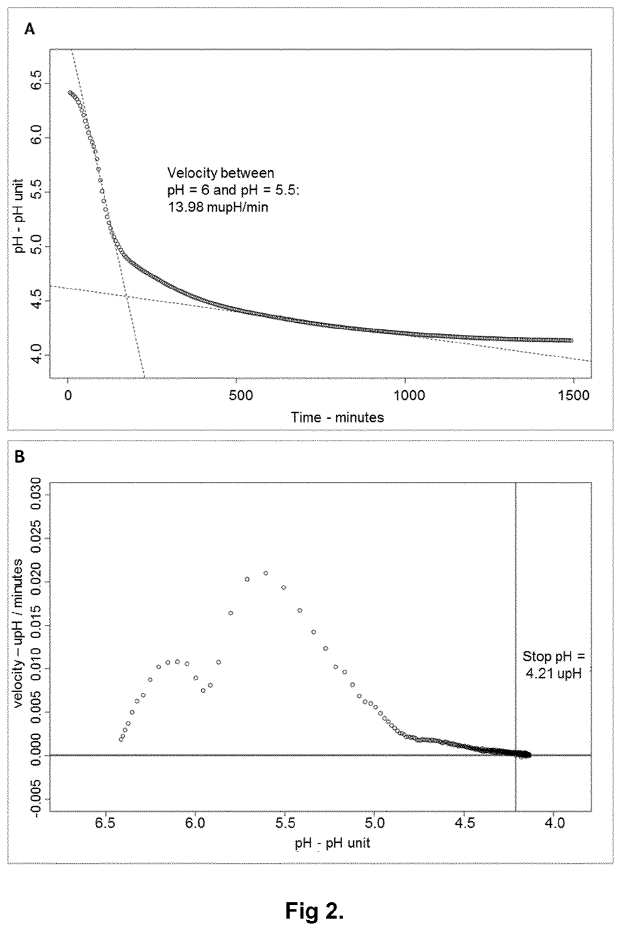

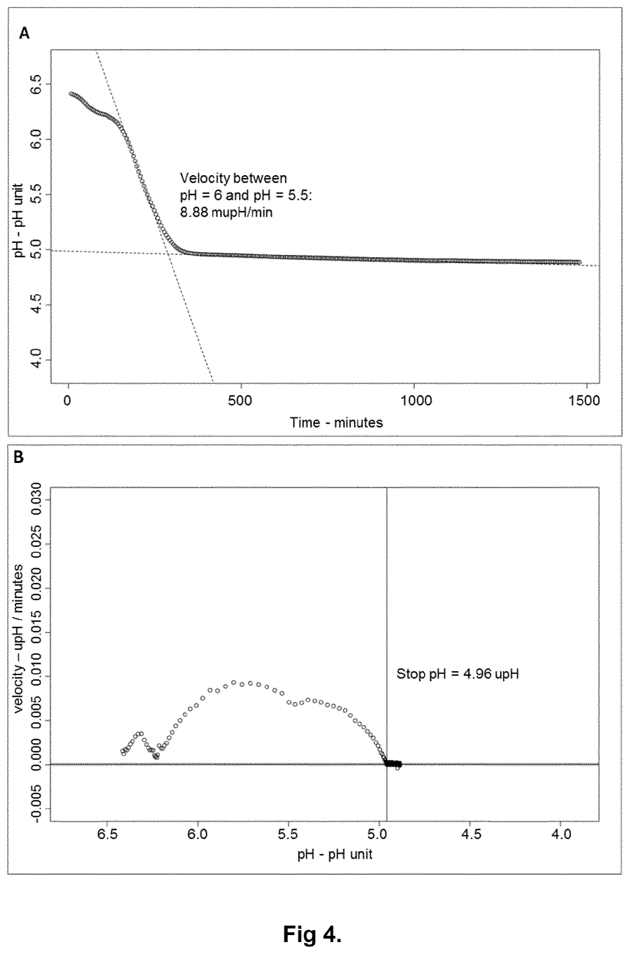

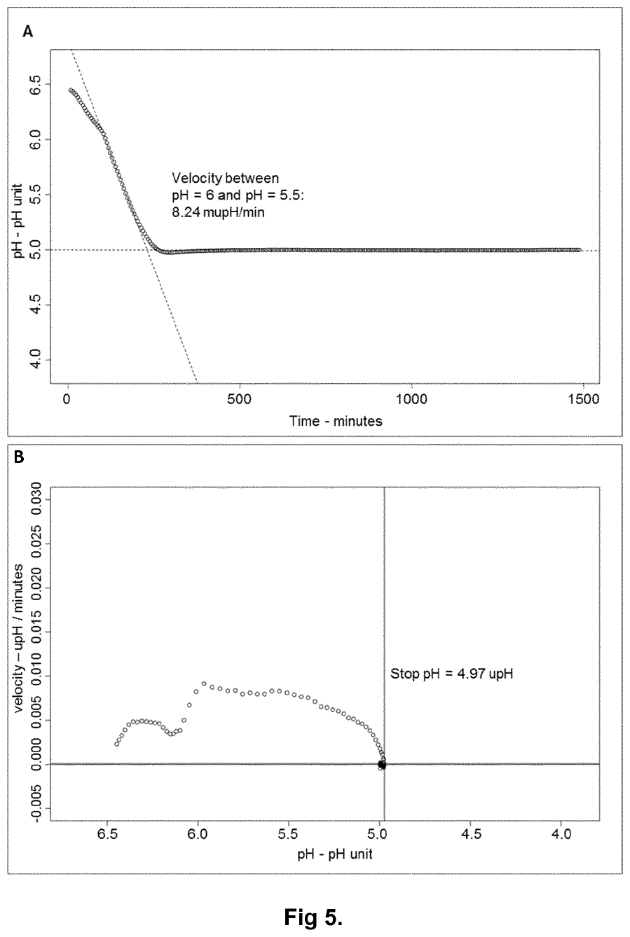

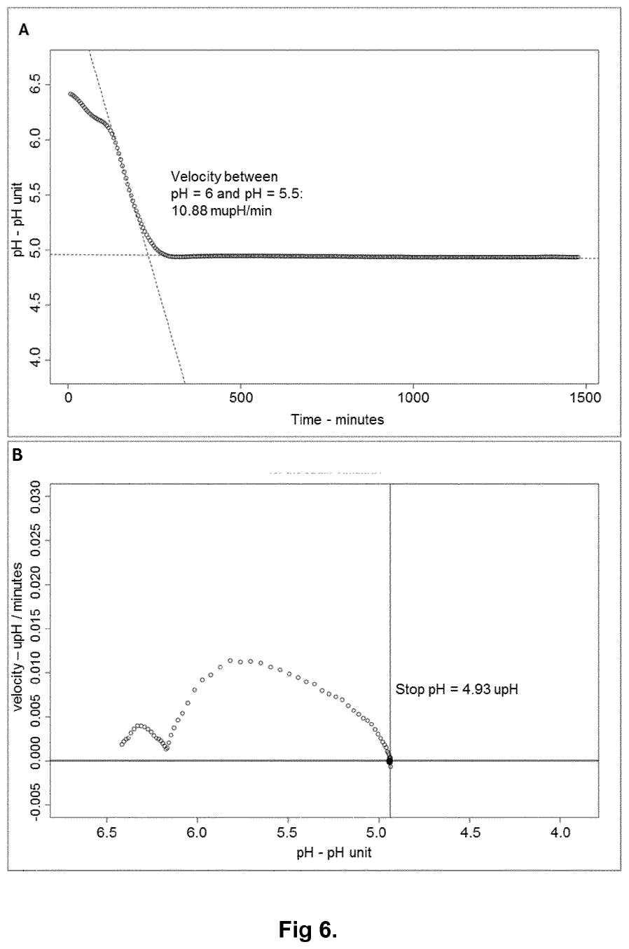

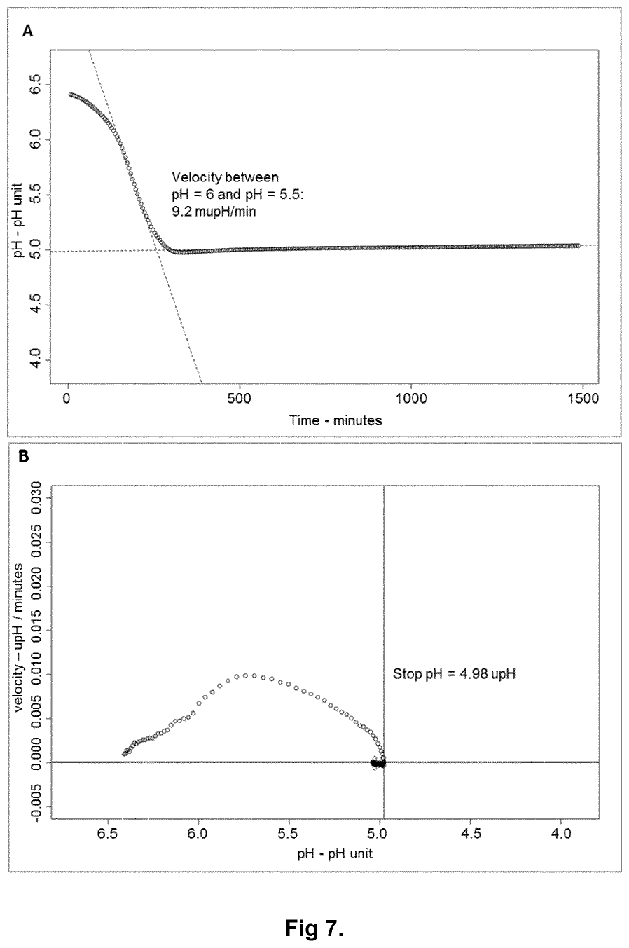

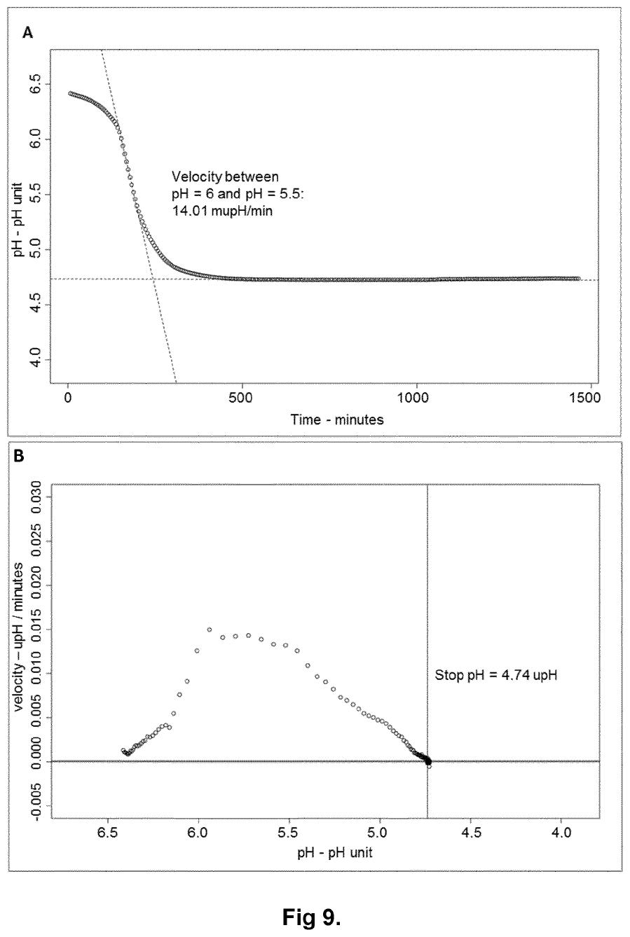

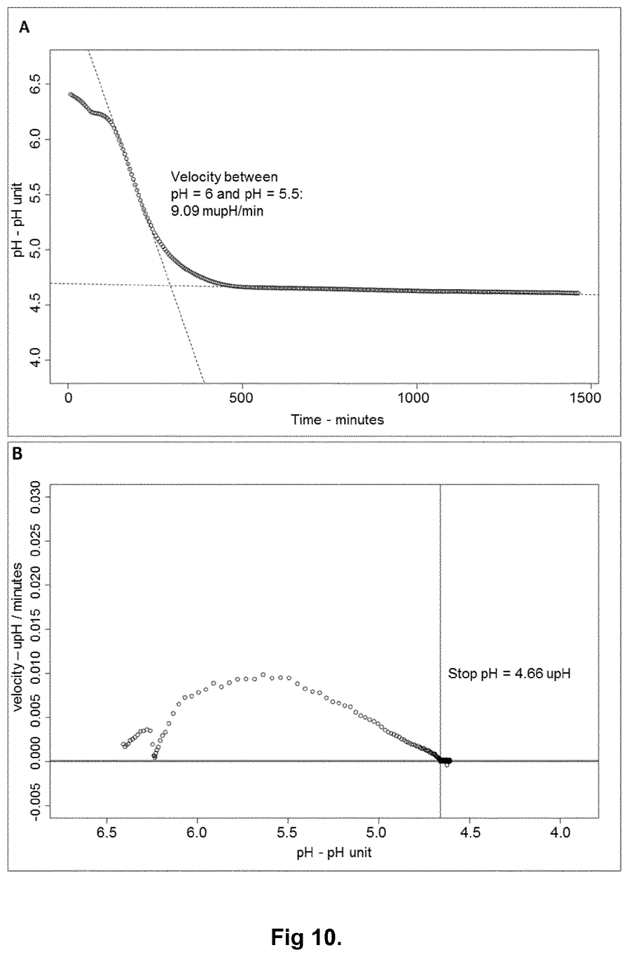

The pH was recorded using a CINAC device. The evolution of the pH over time is represented in FIGS. 2A, 3A, 4A, 5A, 6A, 7A, 8A, 9A and 10A. The velocity between pH 6 and pH 5.5 was calculated as the slope of the linear model deduced from the evolution of the pH as a function of time for value of pH between 6 and 5.5. The slope value is the opposite of the velocity (Table 10). The evolution of the velocity as a function of the pH was also represented (FIGS. 2B, 3B, 4B, 5B, 6B, 7B, 8B, 9B and 10B). Velocity is determined as the instantaneous derivative of the pH evolution as a function of time. The pH_{STOP} was characterized as the pH value at which the speed decrease became non-detectable (below 0.1 mupH/minutes) (Table 10). The time corresponding to the pH_{STOP} (TpH_{STOP}) was also determined (Table 10).

TABLE 10
Acidification kinetics obtained with double and triple
mutated strains and their parental strains

| | | Slope pH 6-5.5 | TpH_{STOP} |
|---|---|---|---|
| Strain | pH_{STOP} | (UpH/min) | (min) |
| DGCC7710 | 4.21 | −0.0140 | 952 |
| ST1m-glcK+ manM | 4.95 | −0.0104 | 392 |
| ST1m-ccpA+ manL | 4.96 | −0.0089 | 388 |
| ST1m-ccpA+ manM | 4.97 | −0.0082 | 298 |
| ST1m-ccpA+ manN | 4.93 | −0.0109 | 312 |
| ST1m-glcK+ ccpA+ manM | 4.98 | −0.0092 | 328 |
| ST1.1 | 4.31 | −0.0180 | 602 |
| ST1.1m-glcK+ manM | 4.74 | −0.0140 | 458 |
| ST1.1m-ccpA+ manL | 4.66 | −0.0091 | 512 |

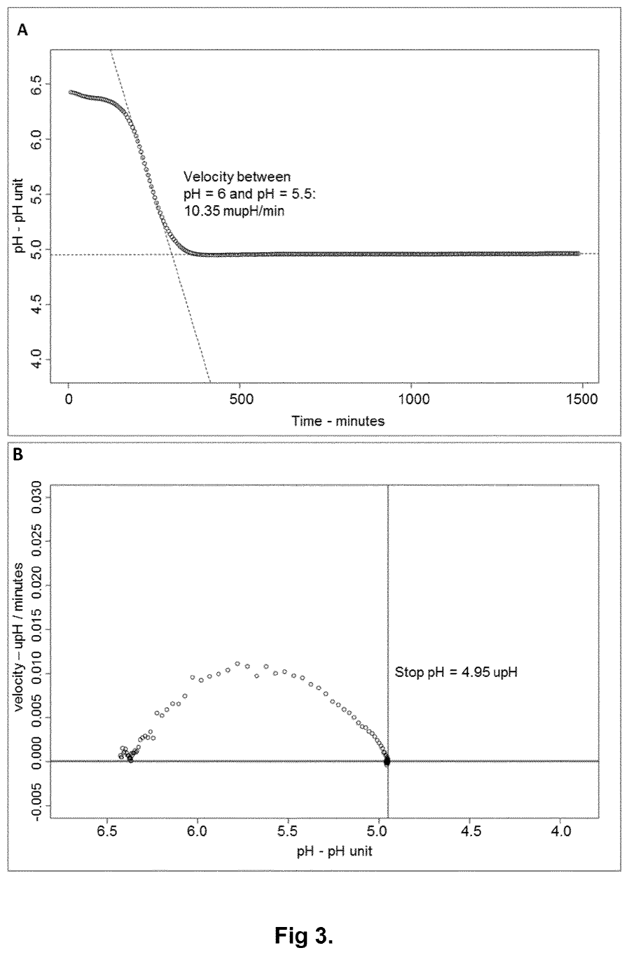

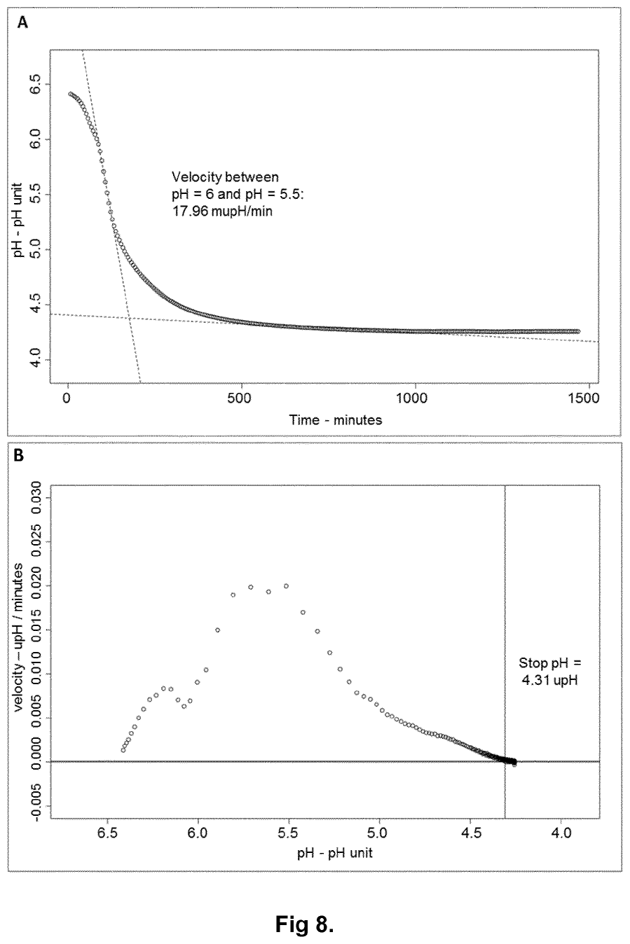

The data of Table 10 show that a milk fermented with the double or triple mutant strains (of the invention) has a higher pH_{STOP} (between 4.66 and 4.98) than a milk fermented with the parental strains (4.21 and 4.31). In other words, this means that the pH obtained at the end of the test B (24h at fermentation temperature) is higher using the double or triple mutant strains (of the invention) than the parental strains. This Table also shows that the pH_{STOP} is obtained between 298 and 512 minutes after inoculation and maintained until 24 hours (i.e., more than 15 hours). FIGS. 3A, 4A, 5A, 6A, 7A, 9A and 10A show the stability of the pH of the fermented milk over time, once the pH_{STOP} was obtained (with the double or triple mutant strains), as compared to a pH regularly decreasing when using the parental strains (FIGS. 2A and 8A). FIGS. 3B, 4B, 5B, 6B, 7B, 9B and 10B show that the speed of acidification (velocity) stops at a high pH between 4.6 and 5 with the double or triple mutant strains, whereas the speed of acidification stops at a low pH (4.21 and 4.31) when using the parental strains (FIGS. 2B and 8B). Altogether, these results show the absence of post-acidification of milk fermented with the double or triple mutant strains of the invention.

These data also show that the acidification kinetics (slope between pH 6 and 5.5) is acceptable at the industrial level to manufacture fermented dairy products.

Altogether, these results show the interest of the double or triple mutant strains for manufacturing producers desiring, not only to obtain fermented products the pH of which is stopped at a higher range (4.6-5.3), but also to maintain their process at fermentation temperature without impacting the pH of the fermented dairy products.

SEQUENCES

SEQ ID NO: 2
MSKKLLGIDLGGTTVKFGILTADGEVQEKWAIETNTFENGSHIVPDIVESLKHRLELYGLTAEDFIGIGMGSPGA
VDRENKTVTGAFNLNWAETQEVGSVIEKELGIPFAIDNDANVAALGERWVGAGANNRNVVFITLGTGVGGGVIAD
GNLIHGVAGAGGEIGHIIVEPDTGFECTCGNKGCLETVASATGIVRVAHHLAEKYEGNSSIKAAVDNGEFVTSKD
IIVAATEGDKFADSIVDKVSKYLGLATANISNILNPDSVVIGGGVSAAGEFLRSRVEGYFTRYAFPQVRRTTKVK
LAELGNDAGIIGAASLAYSIDK
SEQ ID NO: 22
MSKKLLGIDLGGTTVKFGILTADGEVQEKWAIETNTFENGSHIVPDIVESLKHRLELYGLTAEDFIGIGMGSPGA
VDRENKTVTGAFNLNWAETQEVGSVIEKELGIPFAIDNDANVAALGERWVGAGANNRNVVFITLGTGVGGGVIAD
GNLIHGVAGAGGEIGHIIVEPDTGFECTCGNKGCLETVASATGIVRVAHHLAEKYEGNSSIKAAVDNGEFVTSKD
IIVAATEGDKFADSIVDKVSKYLGLATANISNILNPDSVVIGGGVSAAGKFLRSRVEGYFTRYAFPQVRRTTKVK
LAELGNDAGIIGAASLAYSIDK
SEQ ID NO: 45
MSKKLLGIDLGGTTVKFGILTADGEVQEKWAIETNTFENGSHIVPDIVESLKHRLELYGLTAEDFIGIGMGSPGA
VDRENKTVTGAFNLNWAETQEVGSVIEKELGIPFAIDNDANVAALGERWVGAGANNRNVVFITLGTGVSGGVIAD
GNLIHGVAGAGGEIGHIIVEPDTGFECTCGNKGCLETVASATGIVRVAHHLAEKYEGNSSIKAAVDNGEFVTSKD
IIVAATEGDKFADSIVDKVSKYLGLATANISNILNPDSVVIGGGVSAAGEFLRSRVEGYFTRYAFPQVRRTTKVK
LAELGNDAGIIGAASLAYSIDK
SEQ ID NO: 23
MSKKLLGIDLGGTTVKFGILTADGEVQEKWAIETNTFENGSHIVPDIVESLKHRLELYGLTAEDFIGIGMGPPGA
VDRENKTVTGAFNLNWAETQEVGSVIEKELGIPFAIDNDANVAALGERWVGAGANNRNVVFITLGTGVGGGVIAD
GNLIHGVAGAGGEIGHIIVEPDTGFECTCGNKGCLETVASATGIVRVAHHLAEKYEGNSSIKAAVDNGEFVTSKD
IIVAATEGDKFADSIVDKVSKYLGLATANISNILNPDSVVIGGGVSAAGEFLRSRVEGYFTRYAFPQVRRTTKVK
LAELGNDAGIIGAASLAYSIDK
SEQ ID NO: 65
ATGAATACTGATGAAACAATCACAATTTATGATGTAGCGCGTGAAGCTGGAGTATCGATGGCAACTGTTTCTCGT
GTTGTAAATGGTAACAAAAACGTAAAAGAAAACACCCGAAAAAAAGTGCTCGAAGTCATTGATCGTTTGGATTAC
CGTCCAAATGCGGTTGCGCGTGGCTTGGCAAGTAAAAAAACAACTACTGTAGGAGTTGTCATTCCAAATATTGTA
AATAGCTATTTTGCTACTCTAGCTAAAGGTATTGATGACATTGCAACCATGTATAAGTATAATATTGTTCTTGCT
TCCAGTGATGATAATGAGGATCATGAAGTTACAGTCATTCATTCTCTAATTTCTAAACAAGTTGATGGTATTATT
TTTATGGGACACCATCTGACAGAAAAAATCCGTGCAGAATTCTCTCGTACCCGTACACCGATTGTTCTAGCAGGA
ACAGTTGATCTCGAACACCAATTACCAAGTGTTAACATCGACTATAAAGCTGCCGTTGAGGATTGTGTAACGCAA
CTTGCTAAAAATAATGAAAAGGTTGCCTTTGTATCAGGACCACTAATTGATGATATTAATGGCAAACTACGTTTG
GCCGGGTATAAGTCTGGACTTGAAAAGAATAATTTGAGCTACAACGAAGGACTTGTCTTTGAAGCTAAATATAGC
TATAAAGACGGCTTTGAGTTAGCACAACGTGTCTTGAACTCTGGTGCCACTGCTGCCTATGTTGGGGAAGATGAA
TTGGCTGCAGGTCTCTTGAATGGCCTCTTTGCTGCAGGCAAATCAGTTCCAGAAGATTTCGAAATCATCACAAGC
AATGATTCACCGGTTACAAGCTACACACGTCCAAACCTTTCTAGTATAAACCATCCTCTCTATGATTTAGGGGCA
GTTAGCATGCGTATGTTGACTAAAATTATGCATAAGGAAGAACTTGAAGATAAAGACGTTATTCTTAATCATGGT
CTAACTTTACGCCAGTCAACAAAATAA
SEQ ID NO: 71
ATGAATACTGATGAAACAATCACAATTTATGATGTAGCGCGTGAAGCTGGAGTATCGATGGCAACTGTTTCTCGT
GTTGTAAATGGTAACAAAAACGTAAAAGAAAACACCCGAAAAAAGTGCTCGAAGTCATTGATCGTTTGGATTACC
GTCCAAATGCGGTTGCGCGTGGCTTGGCAAGTAAAAAAACAACTACTGTAGGAGTTGTCATTCCAAATATTGTAA
ATAGCTATTTTGCTACTCTAGCTAAAGGTATTGATGACATTGCAACCATGTATAAGTATAATATTGTTCTTGCTT
CCAGTGATGATAATGAGGATCATGAAGTTACAGTCATTCATTCTCTAATTTCTAAACAAGTTGATGGTATTATTT
TTATGGGACACCATCTGACAGAAAAAATCCGTGCAGAATTCTCTCGTACCCGTACACCGATTGTTCTAGCAGGAA
CAGTTGATCTCGAACACCAATTACCAAGTGTTAACATCGACTATAAAGCTGCCGTTGAGGATTGTGTAACGCAAC
TTGCTAAAAATAATGAAAAGGTTGCCTTTGTATCAGGACCACTAATTGATGATATTAATGGCAAACTACGTTTGG
CCGGGTATAAGTCTGGACTTGAAAAGAATAATTTGAGCTACAACGAAGGACTTGTCTTTGAAGCTAAATATAGCT
ATAAAGACGGCTTTGAGTTAGCACAACGTGTCTTGAACTCTGGTGCCACTGCTGCCTATGTTGGGGAAGATGAAT
TGGCTGCAGGTCTCTTGAATGGCCTCTTTGCTGCAGGCAAATCAGTTCCAGAAGATTTCGAAATCATCACAAGCA
ATGATTCACCGGTTACAAGCTACACACGTCCAAACCTTTCTAGTATAAACCATCCTCTCTATGATTTAGGGGCAG
TTAGCATGCGTATGTTGACTAAAATTATGCATAAGGAAGAACTTGAAGATAAAGACGTTATTCTTAATCATGGTC
TAACTTTACGCCAGTCAACAAAATAA
SEQ ID NO: 78
MGIGIIIASHGRFAEGIHQSGSMIFGDQEKVQVVTFMPSEGPDDLYAHFNNAIAQFDVDDEILVLADLWSGSPFN
QASRIARENPDRKIAIITGLNLPMLIQAYTERMMDANATVEQVAANIIKEAKGGIKALPEELNPAEETTAAPVEA
AAPQGAIPEGTVIGDGKLKINLARLDTRLLHGQVVTNWVPYSKADRIIVASDDVAKDELRKELIKQAAPNGIKVN
VVPIQKLIDASKDPRFGNTHALVLFETVQDALRAIEGGVPIKELNVGSMAHSTGKTMVNNVLSMDKDDVACFEKL
RDLGVEFDVRKVPNDSKKDLFELIKKANVQ
SEQ ID NO: 111
ATGGGTATCGGTATTATTATTGCCAGCCATGGTAGGTTCGCTGAAGGAATCCACCAATCAGGCTCTATGATTTTT
GGGGACCAAGAGAAAGTTCAAGTTGTGACTTTCATGCCAAGTGAAGGTCCTGATGATTTGTACGCTCACTTCAAC
AACGCCATTGCACAATTCGATGTTGATGATGAAATTCTTGTTTTGGCTGACCTTTGGAGTGGTTCACCATTTAAC
CAAGCTAGTCGAATCGCTAGGGAAAATCCAGATCGCAAGATTGCTATCATCACAGGACTTAACTTGCCAATGCTA
ATCCAAGCATACACTGAACGTATGATGGATGCTAACGCTACTGTAGAGCAAGTTGCTGCTAATATCATCAAGGAA
GCTAAGGGTGGTATCAAGGCACTTCCAGAAGAGCTAAATCCAGCTGAGGAAACAACTGCAGCTCCTGTAGAAGCT
GCAGCACCTCAAGGAGCTATCCCTGAAGGAACAGTCATCGGAGATGGTAAACTCAAGATTAACTTGGCACGTTTG
GACACACGTCTCTTGCATGGTCAAGTAGTAACTAACTGGGTACCTTATTCTAAAGCAGACCGTATTATTGTTGCT
TCGGATGACGTTGCCAAAGATGAGCTTCGTAAGGAATTGATCAAACAGGCTGCACCAAACGGTATTAAAGTAAAC
GTTGTTCCGATTCAAAAATTAATTGATGCTTCTAAAGACCCACGTTTTGGAAATACACATGCGCTTGTCTTGTTC
GAAACTGTTCAAGACGCACTTCGTGCTATCGAAGGTGGCGTGCCAATAAAAGAACTTAACGTTGGTTCTATGGCT
CACTCAACTGGTAAAACAATGGTTAACAACGTTTTGTCTATGGATAAAGATGATGTTGCTTGTTTTGAAAAATTA
CGTGACCTTGGCGTTTAATTTGACGTCCGTAAGGTTCCAAACGATTCTAAGAAAGATTTGTTTGAGCTTATCAAG
AAAGCTAACGTTCAATAA
SEQ ID NO: 112
MGIGIIIASHGRFAEGIHQSGSMIFGDQEKVQVVTFMPSEGPDDLYAHFNNAIAQFDVDDEILVLADLWSGSPFN
QASRIARENPDRKIAIITGLNLPMLIQAYTERMMDANATVEQVAANIIKEAKGGIKALPEELNPAEETTAAPVEA
AAPQGAIPEGTVIGDGKLKINLARLDTRLLHGQVVTNWVPYSKADRIIVASDDVAKDELRKELIKQAAPNGIKVN
VVPIQKLIDASKDPRFGNTHALVLFETVQDALRAIEGGVPIKELNVGSMAHSTGKTMVNNVLSMDKDDVACFEKL
RDLGV
SEQ ID NO: 130
MSDMSIISAILVVAVAFLAGLESILDQFQFHQPLVACTLIGAATGNLTAGIMLGGSLQMITLAWANIGAAVAPDV
ALASVAAAIILVKGGKFTAEGIGVAIAIAILLAVAGLFLTMPVRTASIAFVHAADKAAEHGNIAGVERAYYLALL
LQGLRIAVPAALLLAIPAQSVQHALGLMPDWLTHGLVVGGGMVVAVGYAMIINMMATREVWPFFAIGFALAAISQ
LTLIALSTIGVAIAFIYLNLSKQGGGNGGGNGGGTSSGSGDPIGDILEDY
SEQ ID NO: 157
ATGTCAGATATGTCAATTATTTCTGCGATTTTGGTCGTAGCTGTTGCCTTCCTTGCTGGTCTTGAAAGTATCCTT
GACCAATTCCAATTCCACCAACCACTTGTTGCATGTACCCTCATCGGTGCTGCCACAGGTAACCTCACTGCAGGT
ATCATGCTTGGTGGTTCTCTTCAAATGATTACCCTTGCTTGGGCAAACATCGGTGCTGCCGTAGCTCCTGACGTT
GCCCTTGCATCTGTTGCCGCTGCCATCATTTTGGTTAAAGGTGGTAAATTTACAGCTGAAGGTATCGGTGTTGCG
ATTGCAATAGCTATCCTGCTTGCAGTTGCAGGTCTCTTCCTAACTATGCCTGTTCGTACAGCATCTATTGCCTTT
GTTCATGCTGCAGATAAAGCTGCAGAACACGGAAACATCGCTGGTGTTGAACGTGCATACTACCTCGCTCTCCTT
CTTCAAGGTTTGCGTATTGCTGTGCCAGCAGCCCTTCTTCTTGCCATCCCGGCCCAATCTGTTCAACATGCCCTT
GGCTTGATGCCTGACTGGCTCACCCATGGTTTGGTTGTCGGTGGTGGTATGGTCGTAGCCGTTGGTTACGCCATG
ATTATCAATATGATGGCTACTCGTTAAGTTTGGCCATTCTTCGCCATTGGTTTTGCTTTGGCAGCAATTAGCCAA
TTGACACTTATCGCTCTTAGTACCATTGGTGTTGCCATCGCCTTCATCTACCTCAACCTTTCTAAACAAGGTGGC
GGAAATGGTGGCGGAAATGGTGGCGGAACTTCATCTGGTTCAGGCGACCCAATCGGCGATATCTTGGAAGACTAC
TAG
SEQ ID NO: 158
MSDMSIISAILVVAVAFLAGLESILDQFQFHQPLVACTLIGAATGNLTAGIMLGGSLQMITLAWANIGAAVAPDV
ALASVAAAIILVKGGKFTAEGIGVAIAIAILLAVAGLFLTMPVRTASIAFVHAADKAAEHGNIAGVERAYYLALL
LQGLRIAVPAALLLAIPAQSVQHALGLMPDWLTHGLVVGGGMVVAVGYAMIINMMATR
SEQ ID NO: 166
MAEKIQLSQADRKKVWWRSQFLQGAWNYERMQNLGWAYSLIPAIKKLYTNKEDQAAALKRHLEFFNTHPYVAAPI
IGVTLALEEEKANGTEIEDAAIQGVKIGMMGPLAGIGDPVFWFTIRPILGALGASLAQAGNIAGPLIFFIGWNLI
RMAFLWYTQELGYKAGSEITKDISGGILKDITKGASILGMFILAVLVERWVSVVFTVKLPGKVLPKGAYIEWPKG
YVTGDQLKTILGQVNDKLSFDKIQVDTLQKQLDSLIPGLTGLLLTFACMWLLKKKVSPITIIIGLFVVGIVASFF
GIM
SEQ ID NO: 206
ATGGCTGAAAAAATTCAATTATCTCAAGCGGATCGTAAAAAAGGTTTGGTGGCGCTCACAATTCTTGCAAGGTGC
ATGGAACTATGAACGTATGCAAAACTTGGGTTGGGCTTACTCACTCATTCCTGCTATCAAAAAACTTTATACTAA
CAAAGAGGACCAAGCCGCAGCTCTTAAACGTCACTTGGAATTCTTCAACACTCACCCTTACGTAGCTGCTCCTAT
CATAGGGGTTACCTTAGCTCTTGAAGAAGAAAAAGCTAATGGTACTGAAATCGAAGATGCGGCTATCCAAGGGGT
TAAAATCGGTATGATGGGTCCACTTGCCGGTATCGGTGACCCTGTCTTCTGGTTCACAATTCGTCCAATTCTTGG
TGCCCTTGGTGCATCATTGGCACAAGCTGGTAACATTGCTGGTCCACTTATCTTCTTCATTGGTTGGAACCTTAT
CCGCATGGCCTTCTTGTGGTACACTCAAGAACTTGGTTACAAAGCAGGTTCAGAAATCACTAAAGACATATCTGG
TGGTATCTTGAAAGATATTACTAAAGGGGCATCAATACTTGGTATGTTCATCTTGGCCGTCCTCGTTGAACGTTG
GGTATCTGTCGTCTTCACTGTAAAGCTTCCAGGTAAAGTTTTGCCTAAAGGTGCTTATATTGAATGGCCAAAAGG
ATATGTTACTGGTGACCAACTAAAAACTATCCTTGGTCAAGTCAACGATAAGCTTAGCTTTGATAAGATTCAAGT
CGATACCCTACAAAAACAATTGGATTCATTAATTCCAGGTTTGACGGGACTTCTCCTTACTTTTGCATGTATGTG
GTTGCTTAAGAAGAAAGTTTCACCAATCACAATCATCATCGGACTCTTTGTAGTTGGTATTGTTGCAAGCTTCTT
CGGAATCATGTAA
SEQ ID NO: 207
MAEKIQLSQADRKKGLVALTILARCMEL

### Strains

DGCC numbers are internal references to DuPont Danisco collection; DSM numbers are the numbers assigned by the Leibniz-Institut DSMZ-Deutsche Sammlung von Mikroorganismen und Zellkulturen, GmbH (Inhoffenstr. 7B, D-38124 Braunschweig), following deposit under the Budapest Treaty.

As far as the Streptococcus thermophilus strain DGCC7710 deposited under the Budapest Treaty at the Leibniz-Institut DSMZ-Deutsche Sammlung von Mikroorganismen und Zellkulturen, GmbH, on Jan. 14, 2014 under number DSM28255 is concerned, we hereby confirm that the depositor, Danisco Deutschland GmbH (of Busch-Johannsen-Strasse 1, D-25899 Niebüll, Germany) has authorised the Applicant (DuPont Nutrition Biosciences ApS) to refer to the deposited biological material in this application. The expressions “DGCC7710 strain” and “DGCC7710 derivative” are used interchangeably with the expressions “DSM28255 strain” and “DSM28255 derivative”.

The Streptococcus thermophilus strain deposited under the Budapest Treaty at the Leibniz-Institut DSMZ-Deutsche Sammlung von Mikroorganismen und Zellkulturen, GmbH, on Aug. 15, 2017 under number DSM32587 has been deposited by DuPont Nutrition Biosciences ApS.

The applicant requests that a sample of the deposited micro-organisms stated herein may only be made available to an expert, until the date on which the patent is granted.

In respect to those designations in which a European Patent is sought, a sample of these deposited microorganisms will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies.

1-30. (canceled)

31. A lactose-positive, galactose-negative, Streptococcus thermophilus strain comprising:
at least one mutated gene encoding a protein of the mannose-glucose-specific phosphotransferase system (mannose-glucose-specific PTS), the glucose import activity of which is decreased or abolished, and
at least one mutated gene selected from the group consisting of:
a) a mutation in a glucokinase (glcK) gene,
b) a mutation in a Catabolite Control Protein A (ccpA) gene; and
c) a combination thereof,
wherein when said strain is fermented in milk, the lactose-positive, galactose-negative, Streptococcus thermophilus strain decreases a lactose concentration by at least 54% compared to a parental strain.

32. The lactose-positive, galactose-negative, Streptococcus thermophilus strain according to claim 31, carrying a mutation in at least one gene encoding a protein of the mannose-glucose-specific PTS and carrying a mutation in a glcK gene such that the glucokinase activity of said strain is reduced but not null.

33. The lactose-positive, galactose-negative, Streptococcus thermophilus strain according to claim 32, wherein
a) the amino acid at position 275 of the glucokinase is not a glutamic acid;
b) the protein sequence of said glucokinase is as defined in SEQ ID NO:25, wherein the amino acid at position 275 of said SEQ ID is not a glutamic acid;
c) the protein sequence of said glucokinase is a GlcK variant sequence having at least 90% similarity or identity with SEQ ID NO:25, wherein the amino acid of the glucokinase corresponding to position 275 of SEQ ID NO:25 (or the amino acid at position 275 of said glucokinase) is not a glutamic acid.

34. The lactose-positive, galactose-negative, Streptococcus thermophilus strain according to claim 32, wherein:
a) the amino acid at position 144 of the glucokinase is not a glycine;
b) the protein sequence of said glucokinase is a sequence as defined in SEQ ID NO:46, wherein the amino acid at position 144 is not a glycine; or
c) the protein sequence of said glucokinase is a GlcK variant sequence having at least 90% similarity or identity with SEQ ID NO:46, wherein the amino acid of said glucokinase corresponding to position 144 of SEQ ID NO:46 or the amino acid at position 144 of said glucokinase is not a glycine.

35. The lactose-positive, galactose-negative, Streptococcus thermophilus strain according to claim 31, wherein the ccpA gene carries a mutation selected from the group consisting of:
a non-sense mutation located between the nucleotide 1 and the nucleotide 270 of the coding sequence of the ccpA gene, and
a mutation located in the first quarter of the coding sequence of the ccpA gene, leading to a frameshift of the open reading frame of the ccpA gene.

36. The lactose-positive, galactose-negative, Streptococcus thermophilus strain according to claim 31, carrying at least one mutated gene encoding a protein of the mannose-glucose-specific phosphotransferase system (mannose-glucose-specific PTS), the glucose import activity of which is decreased or abolished, and carrying a mutation in a Catabolite Control Protein A (ccpA) gene,
wherein the sequence of said mutated ccpA gene is selected from the group consisting of:
a) a sequence as defined in SEQ ID NO:71; and
b) a ccpA variant sequence having at least 90% identity with SEQ ID NO:71.

37. The lactose-positive, galactose-negative, Streptococcus thermophilus strain according to claim 31, wherein the at least one gene encoding a protein of the mannose-glucose-specific PTS is selected from the group consisting of the manL gene, the manM gene and the manN gene.

38. The lactose-positive, galactose-negative, Streptococcus thermophilus strain according to claim 31, wherein the mutated gene encoding a protein of the mannose-glucose-specific PTS codes for a protein, the glucose import activity of which is decreased or abolished, is selected from the group consisting of:
a) a Streptococcus thermophilus IIAB^{Man} protein truncated in position 305 (IIAB^{Man}₃₀₅);
b) a Streptococcus thermophilus IIC^{Man} protein truncated in position 208 (IIC^{Man}₂₀₈); and
c) a Streptococcus thermophilus IID^{Man} protein truncated in position 28 (IID^{Man}₂₈).

39. The lactose-positive, galactose-negative, Streptococcus thermophilus strain according to claim 38, wherein the protein sequence of said IIAB^{Man}₃₀₅ protein is selected from the group consisting of:
a) a sequence as defined in SEQ ID NO:112; and
b) a IIAB^{Man} variant sequence having at least 90% sequence similarity or identity with SEQ ID NO:112;
wherein the protein sequence of said IIC^{Man}₂₀₈ is selected from the group consisting of:
a) a sequence as defined in SEQ ID NO:158; and
b) a IIC^{Man} variant sequence having at least 90% sequence similarity or identity with SEQ ID NO:158;
wherein the protein sequence of said IID^{Man}₂₈ protein is selected from the group consisting of:
a) a sequence as defined in SEQ ID NO:207; and
b) a IID^{Man} variant sequence having at least 90% sequence similarity or identity with SEQ ID NO:207.

40. A composition comprising at least one lactose-positive, galactose-negative, Streptococcus thermophilus strain according to claim 31 in combination with one or more strain(s) selected from the group consisting of a strain of the Lactobacillus genus, a strain of the Lactococcus genus, or a strain of the Bifidobacterium genus.

41. The composition of claim 40, wherein the strain of the Lactobacillus genus is Lactobacillus delbrueckii subsp bulgaricus strain.

42. The composition of claim 40, wherein the strain of the Lactococcus genus is a Lactococcus lactis strain.

43. A method for manufacturing a fermented dairy product comprising:
inoculating a milk substrate with the lactose-positive, galactose-negative, Streptococcus thermophilus strain according to claim 31 or a composition comprising at least one lactose-positive, galactose-negative, Streptococcus thermophilus strain according to claim 31 and
fermenting said inoculated milk, to obtain a fermented dairy product.

44. A fermented dairy product comprising at least one lactose-positive, galactose-negative, Streptococcus thermophilus strain according to claim 31.


SEQUENCE LISTING (222 sequences)

SEQ ID NO: 1 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 2 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 3 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 4 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 5 - DNA, 969 bp, Streptococcus thermophilus
  source 1..969: mol_type = other DNA
atgagtaagaaactcttaggtattgaccttggtggaacaactgttaagtttggtattttgaatgcagatggtgaagttcaagaaaaatgggctattggaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgtcatcgttgatggtaaattaattcatggtgttgccggtgctggtggggaaattggtcacattattgttgaacctgacacaggatttgagtgtacttgcggaaacaaggggtgtctggaaactgtagcttcagcaacaggtattgtacgtatagcacatcatttggcagaaaaatacgaaggaaactcttctattaaagctgctgtagacaatggtgagtttgtgacaagtaaagatattctcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagccgctagtcttgcttatagtattgacaaataa

SEQ ID NO: 6 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 7 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagcttttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtagctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 8 - protein, 322 aa, Streptococcus thermophilus
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 9 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgcagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 10 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 11 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtagctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaatgggtgtctggaaactgtagcttcagcaacaggtattgtacgtgtaacacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 12 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 13 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagactctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagcttgtgacaagtaaagatattctcgtagctgctactgaaggtgataagtttgctgacagcattgttgataaagtctctaaatacctcggacttgcaatagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 14 - protein, 322 aa, Streptococcus thermophilus
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 15 - DNA, 969 bp, Streptococcus thermophilus
  source 1..969: mol_type = other DNA
atgagtaagaaactcttaggtattgaccttggtggaacaactgttaagtttggtattttgaatgcagatggtgaagttcaagaaaaatgggctattggaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgtcatcgttgatggtaaattaattcatggtgttgccggtgctggtggggaaattggtcacattattgttgaacctgacacaggatttgagtgtacttgcggaaacaaggggtgtctggaaactgtagcttcagcaacaggtattgtacgtatagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 16 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 17 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgataatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggttctgttattgaaaaagaacttggtattccattcgctattgataatgatgctaatgtggctgcactgggtgaacgttgggttggtgctggtgctaacagtcgggatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagcttgtgacaagtaaagatattctcgtagctgctactgaaggtgataagtttgctgacagcattgttgataaagtctctaaatacctcggacttgcaatagcaaacatctcaaacattcttaacccagattctgttgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 18 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 19 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgataatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctattattgaaaaagaacttggtattccattcgctattgataatgatgctaatgtggctgcactgggtgaacgttgggttggtgctggtgctaacaatcgggatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagcttgtgacaagtaaagatattctcgtagctgctactgaaggtgataagtttgctgacagcattgttgataaagtctctaaatacctcggacttgcaatagcaaacatctcaaacattcttaacccagattctgtcattatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 20 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGEFLRSRVEGYFTRYAFPQVRRTTKVKLAELGNDAGIIGAASLAYSIDK

SEQ ID NO: 21 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggaaaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 22 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 23 - protein, 322 aa, Streptococcus thermophilus
MSKKLLGIDLGGTTVKFGILTADGEVQEKWAIETNTFENGSHIVPDIVESLKHRLELYGLTAEDFIGIGMGPPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 24 - DNA, 147 bp, Streptococcus thermophilus
  source 1..147: mol_type = other DNA
catagtatcctcctcatatttcagtataacataacttttattttttacctatattttactaaaaaaatagtaaaaatattgattttccatgtgaaaggggttatgatttcagtataaacaaaaagaataagtgagatacatcctatg

SEQ ID NO: 25 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 26 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 27 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 28 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 29 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 30 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 31 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGXFLRSRVEGYFTRYAFPQVRRTTKVKLAELGNDAGIIGAASLAYSIDK

SEQ ID NO: 32 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 33 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGXFLRSRVEGYFTRYAFPQVRRTTKVKLAELGNDAGIIGAASLAYSIDK

SEQ ID NO: 34 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGXFLRSRVEGYFTRYAFPQVRRTTKVKLAELGNDAGIIGAASLAYSIDK

SEQ ID NO: 35 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 36 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 37 - protein, 322 aa, Streptococcus thermophilus
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 38 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 39 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 40 - protein, 322 aa, Streptococcus thermophilus
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGKFLRSRVEGYFTRYAFPQVRRTTKVKLAELGNDAGIIGAASLAYSIDK

SEQ ID NO: 41 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 42 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGKFLRSRVEGYFTRYAFPQVRRTTKVKLAELGNDAGIIGAASLAYSIDK

SEQ ID NO: 43 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGKFLRSRVEGYFTRYAFPQVRRTTKVKLAELGNDAGIIGAASLAYSIDK

SEQ ID NO: 44 - DNA, 969 bp, Streptococcus thermophilus
  source 1..969: mol_type = other DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttagtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 45 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 46 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 47 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 48 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 49 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 50 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 51 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 52 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 53 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 54 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVXGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 55 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVXGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGEFLRSRVEGYFTRYAFPQVRRTTKVKLAELGNDAGIIGAASLAYSIDK

SEQ ID NO: 56 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 57 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 58 - protein, 322 aa, Streptococcus thermophilus
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 59 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 60 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 61 - protein, 322 aa, Streptococcus thermophilus
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 62 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 63 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVSGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 64 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVSGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGEFLRSRVEGYFTRYAFPQVRRTTKVKLAELGNDAGIIGAASLAYSIDK

SEQ ID NO: 65 - DNA, 1002 bp, Streptococcus thermophilus
  source 1..1002: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggccgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 66 - DNA, 1002 bp, Streptococcus thermophilus
  source 1..1002: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgatttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 67 - DNA, 1002 bp, Streptococcus thermophilus
  source 1..1002: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccaaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 68 - DNA, 1002 bp, Streptococcus thermophilus
  source 1..1002: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 69 - DNA, 1002 bp, Streptococcus thermophilus
  source 1..1002: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 70 - DNA, 1002 bp, Streptococcus thermophilus
  source 1..1002: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaatgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 71 - DNA, 1001 bp, Streptococcus thermophilus
  source 1..1001: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggccgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 72 - DNA, 1001 bp, Streptococcus thermophilus
  source 1..1001: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgatttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 73 - DNA, 1001 bp, Streptococcus thermophilus
  source 1..1001: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 74 - DNA, 1001 bp, Streptococcus thermophilus
  source 1..1001: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 75 - DNA, 1001 bp, Streptococcus thermophilus
  source 1..1001: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 76 - DNA, 1001 bp, Streptococcus thermophilus
  source 1..1001: mol_type = other DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaatgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 77 - DNA, 993 bp, Streptococcus thermophilus
  source 1..993: mol_type = other DNA
atgggtatcggtattattattgccagccatggtaggttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaagagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagatttgtttgagcttatcaagaaagctaacgttcaataa

SEQ ID NO: 78 - protein, 330 aa, Streptococcus thermophilus
MGIGIIIASHGRFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 79 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 80 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 81 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaagagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagatttgtttgagcttatcaagaaagctaacgttcaataa

SEQ ID NO: 82 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 83 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaaagccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcacttgtcttgttcgaaactgttcaagacgtacttcgtgctgtcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 84 - protein, 330 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGVEFDVRKVPNDSKKDLFELIKKANVQ

SEQ ID NO: 85 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactatagagcaagttgctgctaatatcatcaaggaagctaagggtggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtgttattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 86 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRVIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 87 - DNA, 993 bp, Streptococcus thermophilus
  source 1..993: mol_type = other DNA
atgggtatcggtattattatcgccagccatggtaagttcgctgaaggaatccaccaatcaggctctatgatttttggggaccaagagaaagttcgagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcgcttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 88 - protein, 330 aa, Streptococcus thermophilus
MGIGIIIASHGKFAEGIHQSGSMIFGDQEKVRVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 89 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 90 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 91 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 92 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 93 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactatagagcaagttgctgctaatatcatcaaggaagctaagggtggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 94 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 95 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgttgctaatatcatcaaggaagctaagggtggtatcaaggcacttccagaggcgctaaatccagctgaggaaacaactgcagctcctgtagaagctgcagcacttcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 96 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVVANIIKEAKGGIKALPEALNPAEETTAAPVEAAALQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 97 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaaagccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattagttgatgcttctaaagacccacgttttggaaatacacatgcacttgtcttgttcgaaactgttcaagacgtacttcgtgctgtcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 98 - protein, 330 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLVDASKDPRFGNTHALVLFETVQDVLRAVEGGVPIKELNVGSMAHSTGKTMVNNVLSMDKDDVACFEKLRDLGVEFDVRKVPNDSKKDLFELIKKANVQ

SEQ ID NO: 99 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgtttttccgattcaaaaattaattgatgcttctaaagacccacgttttggaaatacacatgcgcttgtcttgttcgaaactgttcaagacgcgcttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 100 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVFPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 101 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaataacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaaattgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggactcacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgtcgaatttgacgtccgtaaggttccaaacgattctaagaaagacttgtttgagcttatcaagaaagctaacgttcaataa

SEQ ID NO: 102 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQIAANIIKEAKGGIKALPEELNPAEETTAAPVEAAAPQGAIPEGTVIGDGKLKINLARLDSRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 103 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttactgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 104 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVTANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 105 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaaagccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcgcttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 106 - protein, 330 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 107 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcacttgtcttgttcgaaactgttcaagacgtacttcgtgctgtcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 108 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGVEFDVRKVPNDSKKDLFELIKKANVQ

SEQ ID NO: 109 - DNA, 993 bp, Streptococcus thermophilus
  source 1..993: mol_type = other DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttaccaatgctaatccaagcatacactgaacgtatgatggatgctaacgctactgtagagcaagttgctgctaatatcatcaaggaagctaaggcaggtatcaaggcacttccagaggcgctaaatccagctgaggaaacaactgcagctcctgtagaagctacagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 110 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEATAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 111 - DNA, 993 bp, Streptococcus thermophilus
  source 1..993: mol_type = other DNA
atgggtatcggtattattattgccagccatggtaggttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaagagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgtttaatttgacgtccgtaaggttccaaacgattctaagaaagatttgtttgagcttatcaagaaagctaacgttcaataa

SEQ ID NO: 112 - protein, 305 aa, Streptococcus thermophilus
MGIGIIIASHGRFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 113 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 114 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 115 - protein, 305 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGV

SEQ ID NO: 116 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRVIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 117 - protein, 305 aa, Streptococcus thermophilus
MGIGIIIASHGKFAEGIHQSGSMIFGDQEKVRVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 118 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 119 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 120 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 121 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVVANIIKEAKGGIKALPEALNPAEETTAAPVEAAALQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 122 - protein, 305 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLVDASKDPRFGNTHALVLFETVQDVLRAVEGGVPIKELNVGSMAHSTGKTMVNNVLSMDKDDVACFEKLRDLGV

SEQ ID NO: 123 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVFPIQKLIDASKDPRFGNTHALVLFETVQDALRAIEGGVPIKELNVGSMAHSTGKTMVNNVLSMDKDDVACFEKLRDLGV

SEQ ID NO: 124 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQIAANIIKEAKGGIKALPEELNPAEETTAAPVEAAAPQGAIPEGTVIGDGKLKINLARLDSRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 125 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVTANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 126 - protein, 305 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 127 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGV

SEQ ID NO: 128 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEATAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 129 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 130 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 131 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 132 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 133 - DNA, 828 bp, Streptococcus thermophilus
  source 1..828: mol_type = other DNA
atgtcagatatgtcaattatttctgcgattttggtcgtagctgttgccttccttgctggtcttgaaggtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgctgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 134 - protein, 275 aa, Streptococcus thermophilus
MSDMSIISAILVVAVAFLAGLEGILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 135 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaatctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctctttgtgccattggtgttgccatcgccttcatctacctcaacctttctaaacaaggtggcggaaatggcggcggaaatggtggcggaacttcatctggttcaggcgacccaatcggcgatatcttggaagactactag

SEQ ID NO: 136 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALCAIGVAIAFIYLNLSKQGGGNGGGNGGGTSSGSGDPIGDILEDY

SEQ ID NO: 137 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccattggttttgctttggcagcaattagccaattgacacttatcgctctttgtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 138 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGSGMVVAVGYAMIINMMATREVWPFFAIGFALAAISQLTLIALCAIGVAIAFIYLNLSKQGGGNGGGNGGGTSSGSGDPIGDILEDY

SEQ ID NO: 139 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 140 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALGTIGVAIAFIYLNLSKQGGGNGGGNGGGTSSGSGDPIGDILEDY

SEQ ID NO: 141 - DNA, 828 bp, Streptococcus thermophilus
  source 1..828: mol_type = other DNA
atgtcagatatgtcaattatttctgcgattttggtcgtagctgttgccttccttgctggtcttgaaggtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgctgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 142 - protein, 275 aa, Streptococcus thermophilus
MSDMSIISAILVVAVAFLAGLEGILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALGTIGVAIAFIYLNLSKQGGGNGGGNGGGTSSGSGDPIGDILEDY

SEQ ID NO: 143 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtactctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 144 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALGAIGVAIAFIYLNLSKQGGGNGGGNGGGTSSGSGDPIGDILEDY

SEQ ID NO: 145 - DNA, 828 bp, Streptococcus thermophilus
  source 1..828: mol_type = other DNA
atgtcagatatgtcaattatttctgcgattttggtcgtagctgttgccttccttgctggtcttgaaggtatccttgaccaattccaattccaccaaccacttgttgcatataccctcatcggtgctgccacaggtaacctcactgcaggtatcatgcttggtggttctcttcaaatgattacccttgcttgggcaaacatcggtgctgccgtagctcctgacgttgcccttgcatctgttgctgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 146 - protein, 275 aa, Streptococcus thermophilus
MSDMSIISAILVVAVAFLAGLEGILDQFQFHQPLVAYTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 147 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggcggaaatagtggcggaaatggtggcggaacttcatctggttcaggcgacccaatcggcgatatcttggaagactactag

SEQ ID NO: 148 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATREVWPFFAIGFALAAISQLTLIALGAIGVAIAFIYLNLSKQGGGNSGGNGGGTSSGSGDPIGDILEDY

SEQ ID NO: 149 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattacctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctctttgtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 150 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASITFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALCAIGVAIAFIYLNLSKQGGGNGGGNGGGTSSGSGDPIGDILEDY

SEQ ID NO: 151 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctcttggtgccattggtgttgccatcgccttcatctacctcaacctttctaaacatggtggcggaaatggtggcggaaatggtggcggaacttcatctggttcaggcgacccaatcggcgatatcttggaagactactag

SEQ ID NO: 152 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALGAIGVAIAFIYLNLSKHGGGNGGGNGGGTSSGSGDPIGDILEDY

SEQ ID NO: 153 - DNA, 828 bp, Streptococcus thermophilus
  source 1..828: mol_type = other DNA
atgtcagatatgtcaattatttctgcgattttggtcgtagctgttaccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggtggaaatggtggcggaaatggtggcggaacttcatctggttcaggcgacccaatcggcgatatcttggaagactactag

SEQ ID NO: 154 - protein, 275 aa, Streptococcus thermophilus
MSDMSIISAILVVAVTFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATREVWPFFAIGFALAAISQLTLIALGAIGVAIAFIYLNLSKQGGGNGGGNGGGTSSGSGDPIGDILEDY

SEQ ID NO: 155 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 156 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATREVWPFFAIGFALAAISQLTLIALGAIGVAIAFIYLNLSKQGGGNGGGNGGGTSSGSGDPIGDILEDY

SEQ ID NO: 157 - DNA, 828 bp, Streptococcus thermophilus
  source 1..828: mol_type = other DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgttaagtttggccattcttcgccattggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 158 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATR

SEQ ID NO: 159 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATR

SEQ ID NO: 160 - protein, 208 aa, Streptococcus thermophilus
MSDMSIISAILVVAVAFLAGLEGILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATR

SEQ ID NO: 161 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGSGMVVAVGYAMIINMMATR

SEQ ID NO: 162 - protein, 208 aa, Streptococcus thermophilus
MSDMSIISAILVVAVAFLAGLEGILDQFQFHQPLVAYTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATR

SEQ ID NO: 163 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATR

SEQ ID NO: 164 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASITFVHAADKAAEHGNIAGVERAYYLSLLLQGLRIAVPAALLLAIPAQSVQHALGLMPDWLTHGLVVGGGMVVAVGYAMIINMMATR

SEQ ID NO: 165 - protein, 208 aa, Streptococcus thermophilus
MSDMSIISAILVVAVTFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATR

SEQ ID NO: 166 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 167 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 168 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgttttctggttcacaattcgtccaattcttggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactattcttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 169 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 170 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattattgcaagcttcttcggaatcatgtaa

SEQ ID NO: 171 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIIASFFGIM

SEQ ID NO: 172 - DNA, 912 bp, Streptococcus thermophilus
  source 1..912: mol_type = other DNA
atgtctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaatctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 173 - protein, 303 aa, Streptococcus thermophilus
MSEKIQLSQADRKKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQSGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 174 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggttcatggaactatgaacgtatgcaaaacttgggttgggcttactcactcattcccgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcagctatccaaggggttaaaatcggtatgatgggtccacttgccggtatcggtgaccctgtcttctggttcacaattcgtccaattcttggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaagtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaattacaatcatcatcggactctttgtagttggtattgttgcaagcttcttcggaatcatgtaa

SEQ ID NO: 175 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAVLVERWVSVVFTVKLPGKVLPKSAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 176 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtattggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 177 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 178 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgccattggtgcttcattggcacaagctggtaacattgctggtccacttatcttcttcattggttggaaccttatccgcatggtcttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataatattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 179 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMVFLWYTQELGYKAGSEITKDMSGGILKDITKGASILGMFILAVLVERWVSVVFTVKLPGKVLPKGAYIEWPKGYVTGDQLKTILGQVNDKLSFDNIQVDTLQKQLDSLIPGLTGLLLTFACMWLLKKKVSPITIIIGLFVVGIVASFFGIM

SEQ ID NO: 180 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagtttcttcggaatcatgtaa

SEQ ID NO: 181 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 182 - DNA, 912 bp, Streptococcus thermophilus
  source 1..912: mol_type = other DNA
atgtctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 183 - protein, 303 aa, Streptococcus thermophilus
MSEKIQLSQADRKKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 184 - DNA, 912 bp, Streptococcus thermophilus
  source 1..912: mol_type = other DNA
atggctgaaaaaattcaattatctcaagcggatcgtagaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggagttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattactggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 185 - protein, 303 aa, Streptococcus thermophilus
MAEKIQLSQADRRKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNITGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 186 - DNA, 912 bp, Streptococcus thermophilus
  source 1..912: mol_type = other DNA
atggctgaaaaaattcaattatctcaagcggatcgtagaaaggtttggtggcgcttacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactctagaacttggttacaaagaaggttcagaaatcactaaagacatgtctggtggtatcttgaaagatattactaaaggggcatcaatacttggtatgttcttcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 187 - protein, 303 aa, Streptococcus thermophilus
MAEKIQLSQADRRKVWWRLQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMAFLWYTLELGYKEGSEITKDMSGGILKDITKGASILGMFFLAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 188 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccttcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 189 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAFLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 190 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatggggattaccttagctcttgaagaagaaaaagctaatggtactgaaatcgaagatgcggctatccaaggggttaaaatcggtatgatgggtccacttgccggtatcggtgaccctgtcttctggttcacaattcgtccaattctaggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcactaatcacaatcatcatcggactctttgtagttggtattgttgcaagcttcttcggaatcatgtaa

SEQ ID NO: 191 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGITLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSLITIIIGLFVVGIVASFFGIM

SEQ ID NO: 192 - DNA, 912 bp, Streptococcus thermophilus
  source 1..912: mol_type = other DNA
atggctgaaaaaattcaattatctcaagcggatcgtagaaaggtttggtggcgctcacaattcttgcaaggttcatggaactatgaacgtatgcaaaacttgggttgggcttactcactcattcctgctatcaaaaaactttatactaacaaagaggatcaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccccttgcaggtatcggtgaccctgtcttctggttcacaattcgtccaattcttggtgcccttggtgcatcattggcacaagctggtaacattactggtccacttatcttcttcattggttggaaccttatccgcatggccttcttgtggtacactcaagaacttggttacaaatcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 193 - protein, 303 aa, Streptococcus thermophilus
MAEKIQLSQADRRKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNITGPLIFFIGWNLIRMAFLWYTQELGYKSGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 194 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaatctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 195 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQSGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 196 - DNA, 912 bp, Streptococcus thermophilus
  source 1..912: mol_type = other DNA
atgtctgaaaaaattcaattatctcaagcggatcataaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaatctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 197 - protein, 303 aa, Streptococcus thermophilus
MSEKIQLSQADHKKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQSGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 198 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatagaggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 199 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIEVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 200 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagatgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagtttcttcggaatcatgtaa

SEQ ID NO: 201 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVDAPIMGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 202 - DNA, 912 bp, Streptococcus thermophilus
  source 1..912: mol_type = other DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 203 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 204 - DNA, 912 bp, Streptococcus thermophilus
  source 1..912: mol_type = other DNA
atggctgaaaaaattcaattatctcaagcggatcgtagaaaggtttggtggcgctcacaattcttgcaaggttcatggaactatgaacgtatgcaaaacttgggttgggcttactcactcattcctgctatcaaaaaactttatactaacaaagaggatcaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgcaggtatcggtgaccctgtcttctggttcacaattcgtccaattcttggtgccattggtgcttcattggcacaagctggtaacattgctggtccacttatcttcttcattggttggaaccttatccgcatggccttcttgtggtacactcaagaacttggttacaaatcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 205 - protein, 303 aa, Streptococcus thermophilus
MAEKIQLSQADRRKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKSGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 206 - DNA, 913 bp, Streptococcus thermophilus
  source 1..913: mol_type = other DNA
atggctgaaaaaattcaattatctcaagcggatcgtaaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 207 - protein, 28 aa, Streptococcus thermophilus
MAEKIQLSQADRKKGLVALTILARCMEL

SEQ ID NO: 208 - protein, 28 aa, Streptococcus thermophilus
MAEKIQLSQADRKKGLVALTILARFMEL

SEQ ID NO: 209 - protein, 28 aa, Streptococcus thermophilus
MSEKIQLSQADRKKGLVALTILARFMEL

SEQ ID NO: 210 - protein, 28 aa, Streptococcus thermophilus
MAEKIQLSQADRRKGLVALTILARFMEL

SEQ ID NO: 211 - protein, 28 aa, Streptococcus thermophilus
MSEKIQLSQADHKKGLVALTILARFMEL

SEQ ID NO: 212 - DNA, 21 bp, synthetic construct
  misc_feature 1..21: note = Oligonucleotide primer
  source 1..21: mol_type = other DNA
caggtatgagtttagcaacgg

SEQ ID NO: 213 - DNA, 21 bp, synthetic construct
  misc_feature 1..21: note = Oligonucleotide primer
  source 1..21: mol_type = other DNA
agttcaatcttcatcatctcg

SEQ ID NO: 214 - DNA, 20 bp, synthetic construct
  misc_feature 1..20: note = Oligonucleotide primer
  source 1..20: mol_type = other DNA
gtagccacattgttcctgac

SEQ ID NO: 215 - DNA, 20 bp, synthetic construct
  misc_feature 1..20: note = Oligonucleotide primer
  source 1..20: mol_type = other DNA
ttgctgaagctacagtttcc

SEQ ID NO: 216 - DNA, 20 bp, synthetic construct
  misc_feature 1..20: note = Oligonucleotide primer
  source 1..20: mol_type = other DNA
taagcaagactagcagctcc

SEQ ID NO: 217 - DNA, 20 bp, synthetic construct
  misc_feature 1..20: note = Oligonucleotide primer
  source 1..20: mol_type = other DNA
ttgcgtagtcgtgttgaagg

SEQ ID NO: 218 - DNA, 21 bp, synthetic construct
  misc_feature 1..21: note = Oligonucleotide primer
  source 1..21: mol_type = other DNA
attgtcccttcataagcatcg

SEQ ID NO: 219 - DNA, 20 bp, synthetic construct
  misc_feature 1..20: note = Oligonucleotide primer
  source 1..20: mol_type = other DNA
cgaactgggtgcagatgatg

SEQ ID NO: 220 - DNA, 19 bp, synthetic construct
  misc_feature 1..19: note = Oligonucleotide primer
  source 1..19: mol_type = other DNA
attcaccacggcctgagac

SEQ ID NO: 221 - DNA, 20 bp, synthetic construct
  misc_feature 1..20: note = Oligonucleotide primer
  source 1..20: mol_type = other DNA
gaagcagtttggggtagtag

SEQ ID NO: 222 - DNA, 20 bp, synthetic construct
  misc_feature 1..20: note = Oligonucleotide primer
  source 1..20: mol_type = other DNA
gagttatctacaggagctgg